(19) United States
(12) Patent Application Publication
(10) Pub. No.: US 2026/0367086 A1
(43) Pub. Date: Nov. 19, 2026

(54) Detection of Cytocapsular Tubes in Tissue for Cancer Metastasis Analysis

(71) Applicant: Cellmig Biolabs Inc., Chestnut Hill, MA (US)

(72) Inventor: Tingfang Yi, Chestnut Hill, MA (US)

(21) Appl. No.: 19/135,106

(22) PCT Filed: Dec. 4, 2023
(86) PCT No.: PCT/US2023/082261

§ 371 Date: Jun. 3, 2025

Related U.S. Application Data
(60) Provisional application No. 63/386,248, filed on Dec. 6, 2022.
(60) Provisional application No. 63/604,962, filed on Dec. 1, 2023.

(51) Int. Cl. G01N 33/575; G01N 33/543; G01N 33/573; G01N 33/68

(52) U.S. Cl. CPC G01N 33/5759; G01N 33/54306; G01N 33/573; G01N 33/6893; G01N 2333/914

(57) ABSTRACT

The present disclosure provides for methods of detecting cytocapsular tubes in a tissue of a subject for cancer diagnosis, screening and metastasis prediction.

### RELATED APPLICATION DATA

This application claims priority to U.S. Provisional Application No. 63/386,248 filed on Dec. 6, 2022 and U.S. Provisional Application No. 63/604,962, filed on Dec. 1, 2023, both of which are hereby incorporated herein by reference in their entirety for all purposes.

### FIELD

The invention is related to the area of detection of cytocapsular tubes in tissue for cancer metastasis analysis.

### BACKGROUND

Cancer is a leading cause of all deaths. Curing cancer is among the most urgent demands of human health care (Cassetta L, Pollard J, A timeline of tumor-associated macrophage biology. Nat Rev Cancer. 23, 238-257 (2023); Chaffer C, Weinberg RA, A Perspective on Cancer Cell Metastasis. Science. 331, 1559-64 (2011); Hanahan D, Weinberg R A, Hallmarks of cancer: the next generation. Cell. 144, 646-74 (2011)). Almost 10 million cancer deaths and around 19.3 million new cancer cases occurred in 2020 alone worldwide (Sung H, et al, Global cancer statistics 2020: GLOBOCAN estimates of incidence and mortality worldwide for 36 cancers in 185 countries. CA Cancer J Clin. Online ahead of print (2021)). The number of new cancer cases and cancer deaths increases yearly worldwide. Cancer metastasis is a major cause of cancer lethality. Cancer metastasis is the process of cancer cell migration from primary sites of origin to secondary sites of neighboring and distant tissues and organs (Friedl P, Wolf K, Tumour-cell invasion and migration: Diversity and escape mechanisms. Nat Rev Cancer. 3, 362-374 (2003); Barbolina M V, et al., Microenvironmental regulation of ovarian cancer metastasis. Cancer Treat Res. 149, 319-334 (2009)).

Cancer is a collection of related diseases that involve abnormal cell growth with the potential to invade or spread to other parts of the body. Cancers include malignant tumors and malignant neoplasia that are characterized by uncontrolled cell proliferation and metastasis. Benign tumors are neoplasia with uncontrolled cell proliferation but without metastasis. There are hundreds of different types and subtypes of cancers based on various classification methods and standards worldwide. A majority of the different types and subtypes of cancers are classified based on the tissue sites or cells of origin, tumor grading, cancer staging, and molecular markers and their combinations (Thiery J P, Acloque H, Huang R Y, Nieto M A, Epithelial-mesenchymal transitions in development and disease. Cell. 139, 871-890 (2009)).

In the last decades, many aspects of cancer progression have been studied intensively. These include sub-clonal, cellular, cellular-plastic, genetic, genomic, proteomic, signaling, metabolic, and tumor microenvironmental topics (Chiang A, Massagué J, Molecular basis of metastasis. N Engl J Med. 359, 2814-23 (2008); Dienstmann R, et al., Personalizing colon cancer adjuvant therapy: selecting optimal treatments for individual patients. J Clin Oncol. 33, 1787-96 (2015); Mody K, Bekaii-Saab T, Clinical Trials and Progress in metastatic Colon Cancer. Surg Oncol Clin N Am. 27, 349-365 (2018); Patras L, et al, Immune determinants of the pre-metastatic niche. Cancer Cell. 41, 546-572 (2023); Turajlic S, Swanton C, Metastasis as an evolutionary process. Science. 352, 169-75 (2016); Vendramin R, et al., Cancer evolution: Darwin and beyond. EMBO J. 40, e108389 (2021); Valastyan S, Weinberg RA, Tumor metastasis: molecular insights and evolving paradigms. Cell. 147, 275-292 (2011)). Several models have been hypothesized for tumor progression, which invoke diverse scales of driving factors including genetic alterations, genomic aberrations (discordant inheritance, DNA macro-alterations), oncoprotein promotion, signaling pathways, cell plasticity, intercellular reactions, and microenvironments (Erler J, et al., Lysyl oxidase is essential for hypoxia-induced metastasis. Nature. 440, 1222-6 (2006); Kannarkatt J, et al., Adjuvant Chemotherapy for Stage II Colon Cancer: A Clinical Dilemma. J Oncol Pract. 13, 233-241 (2017); Koo M, et al., Conceptual Framework to Guide Early Diagnosis Programs for Symptomatic Cancer as Part of Global Cancer Control, JCO Glob Oncol. 7, 35-45 (2021); Nowell P, The clonal evolution of tumor cell populations. Science. 194, 23-28 (1976); Ring A, et al., Biology, vulnerabilities and clinical applications of circulating tumour cells. Nat Rev Cancer. 23, 95-111 (2023); Seferbekova Z, et al., Spatial biology of cancer evolution. Nat Rev Genet. 24, 295-313 (2023)). Tumors' biological, biochemical, biophysical and metabolic features are targeted for the invention of numerous types of cancer diagnosis and therapies (Friedl P, Wolf K, Tumour-cell invasion and migration: Diversity and escape mechanisms. Nat Rev Cancer. 3, 362-374 (2003); Huerta S, et al., Colon cancer and apoptosis. Am J Surg. 191, 517-26 (2006); Labianca R, et al., Colon cancer. Crit Rev Oncol Hematol. 74, 106-33 (2010); Visser K, et al., The evolving tumor microenvironment: From cancer initiation to metastatic outgrowth. Cancer Cell. 41, 374-403 (2023)).

The outcome of cancer treatment and patient survival rate depends on accurate diagnosis, prognosis, metastasis prediction and evaluation of cancer. However, a uniform and accurate prediction of cancer metastasis grade, a key characteristic of cancer and a major cause of cancer death, is lacking in clinical pathology assays. Further, the current clinical cancer treatment outcomes suggest that the mechanisms underlying cancer development and progression in vivo have not been fully resolved (Gerstberger S, et al., Metastasis. Cell. 186, 564-1579 (2023); Iqbal A, George T J, Randomized Clinical Trials in Colon and Rectal Cancer. Surg Oncol Clin N Am. 26, 689-704 (2017); Schiffman J, et al., Early detection of cancer: past, present, and future. Am Soc Clin Oncol Educ Book. 57-65 (2015)).

There is a continuing need in the art for methods that allow for accurate diagnosis, prognosis and metastasis prediction and evaluation of cancer.

### SUMMARY

The present disclosure addresses this need and is based on the surprising discovery that endogenous plasma membrane Ca²⁺-ATPases (PMCAs) are highly expressed in the membranes of cytocapsular tubes in cancer tissues. Using anti-PMCA antibodies, cytocapsular tubes were detected in cancer tissues but not in normal tissues or benign tissues.

According to one aspect, the present disclosure provides a method of detecting cytocapsular tubes in a tissue of a subject. In one embodiment, the method includes obtaining a tissue from the subject, and detecting the presence of the cytocapsular tubes in the tissue by contacting the tissue with an anti-plasma membrane Ca²⁺-ATPase (PMCA) antibody.

According to another aspect, the present disclosure provides a method of predicting the grade of cancer metastasis of a subject. In one embodiment, the method includes obtaining a tissue from the subject, and detecting the presence of the cytocapsular tubes in the tissue by contacting the tissue with an anti-plasma membrane Ca²⁺-ATPase (PMCA) antibody. In one embodiment, the presence of the cytocapsular tubes in the tissue is associated with metastasis cancer. In certain embodiments, the density and morphology of the cytocapsular tubes detected in the tissue is used to predict the grade of cancer metastasis of the subject. In one embodiment, increase in the density of cytocapsular tubes is associated with more advanced grade of cancer metastasis. In another embodiments, thinner and cloud-like cytocapsular tube morphology and more degradation is associated with more advanced grade of cancer metastasis.

According to still another aspect, the present disclosure provides a method of screening a subject for cancer. In one embodiment, the method includes obtaining a tissue from the subject, and detecting the presence of the cytocapsular tubes in the tissue by contacting the tissue with an anti-plasma membrane Ca²⁺-ATPase (PMCA) antibody. In one embodiment, the presence of the cytocapsular tubes in the tissue is an indication that the tissue is cancerous. In another embodiment, the absence of the cytocapsular tubes in the tissue is an indication that the tissue is healthy or benign.

According to one aspect, the present disclosure provides a method of detecting cytocapsular tubes in a tissue sample from a subject. In one embodiment, the method includes contacting the tissue sample with an antibody, wherein the antibody binds to plasma membrane Ca²⁺-ATPase (PMCA) in cytocapsular membrane of the cytocapsular tubes, and detecting the presence of the cytocapsular tubes in the tissue sample by detecting the antibody. In another embodiment, the method includes detecting cytocapsulars, cytocapsulasome vesicles, cytocapsular oncocells, cytocapsular tumorspheres, and cytocapsular tumorsphere network systems in the tissue sample. The presence of the cytocapsular tubes and/or cytocapsulars in the tissue sample indicates the presence of or risk for (i.e. a potential for a development of) a cancer disease in the subject.

According to another aspect, the present disclosure provides a method of detecting cytocapsular tubes in vitro. In one embodiment, the method includes contacting the cytocapsular tubes in a 3D matrix culture with an antibody, wherein the antibody binds to plasma membrane Ca²⁺-ATPase (PMCA) in cytocapsular membrane of the cytocapsular tubes, and detecting the presence of the cytocapsular tubes in the 3D matrix culture by detecting the antibody. In another embodiment, the method includes detecting cytocapsulars, cytocapsulasome vesicles, cytocapsular oncocells, cytocapsular tumorspheres, and cytocapsular tumorsphere network systems in the 3D matrix culture.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-1F depict representative H&E staining images of clinical normal breast tissue (FIG. 1A), benign breast tumor tissue (FIG. 1B) and breast cancer tissue (FIG. 1C) and representative immunohistochemistry (IHC) fluorescence staining images of clinical normal breast tissue (FIG. 1D), benign breast tumor tissue (FIG. 1E) and breast cancer tissue (FIG. 1F) with anti-PMCA2 rabbit polyclonal antibody anti-γ-actin antibodies and DAPI (nucleus). Anti-PMCA2 rabbit polyclonal antibody showed increased expression of PMCA2 in cytocapsular tubes (CCT, white arrows) in clinical malignant breast cancer tissue (FIG. 1F). Breast cancer cells in malignant breast cancer tissue (FIG. 1F), but not in normal breast tissue cells (FIG. 1D) or benign breast tumor tissue (FIG. 1E), generate many long cytocapsular tubes in enhanced green color (tube-shaped morphologies). Scale bar: 10 μm.

FIGS. 2A-2B depict representative H&E staining images of malignant breast cancer in stage II (FIGS. 2A, A′ and A″). Many cancer cells (yellow arrows, in spindle morphology in the sectioned and exposed in dark brown color) were observed to distribute in the curved but smear tissue background. The black framed area is enlarged and shown in the right panel (A″), showing several sectioned and exposed cancer cells (yellow arrows, in dark brown color) in migration. FIG. 2B depict representative immunohistochemistry (IHC) fluorescence staining image of breast cancer in stage II with anti-PMCA2 rabbit polyclonal antibody anti-γ-actin antibodies and DAPI (nucleus). Breast cancer cells in malignant breast cancer tissues were observed to generate many long cytocapsular tubes in enhanced green color (tube-shaped morphologies). Many curved cytocapsular tubes (CCT, white arrows) align together to form bundles, with cancer cells (red arrows, in spindle morphology in the sectioned and exposed nuclei in blue color) in migration inside the CCTs. The white framed area is enlarged and shown in the right panel (FIG. 2B), in which several sectioned and exposed cancer cells (red arrows, in blue color) in migration inside the CCTs (white arrows) were shown. Scale bar: 10 μm.

FIGS. 3A-3B depict representative H&E staining image (FIG. 3A) and immunohistochemistry (IHC) fluorescence staining image (FIG. 3B) of clinical malignant breast cancer in stage III with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows) in clinical malignant breast cancer. Breast cancer cells in malignant breast cancer tissue generate large quantities of long and curved cytocapsular tubes and form CCT masses. Scale bar: 10 μm.

FIGS. 4A-4E depict representative H&E staining (FIG. 4A) and immunohistochemistry (IHC) staining images with antibodies recognizing ER (estrogen receptor, FIG. 4B), PR (Progesterone receptor, FIG. 4C) and HER2 (human epidermal growth factor receptor 2, FIG. 4D). The staining were identified as PR+ (cancer cells in dark brown color)/HER2+ (cancer cells in brown color)/ER− in breast cancer tissue. Anti-PMCA2 rabbit polyclonal antibody anti-γ-actin antibodies and DAPI (nucleus) in IHC (FIG. 4E) showed high levels of PMCA-2 protein expression in cytocapsular tubes (CCT, in curved tube-shaped morphologies with 3-6 μm in diameter/width, white arrow, in red-framed areas). These CCT morphologies will provide additional metastasis information for pathologists. The specimens in the above analyses were five continuous sectioned specimens (5 μm in thickness, FIGS. 4A-4E). Scale bar: 10 μm.

FIGS. 5A-5B depict representative H&E staining image (FIG. 5A) and immunohistochemistry (IHC) fluorescence staining image (FIG. 5B) with anti-PMCA2 rabbit polyclonal antibody anti-γ-actin antibodies and DAPI (nucleus) of clinical malignant prostate cancer. Anti-PMCA2 rabbit polyclonal antibody showed abundant expression of PMCA2 in cytocapsular tubes (CCT, white arrows) in clinical malignant prostate cancer. Prostate cancer cells in malignant prostate tumors generate long and curved cytocapsular tubes that can form large irregular structures. Many CCTs showed thinner strands morphology corresponding to more degraded CCTs (CTS, orange arrows). Scale bar: 10 μm.

FIGS. 6A-6D depict representative immunohistochemistry (IHC) fluorescence staining images of clinical malignant adrenal gland cancer (FIG. 6A), appendix cancer (FIG. 6B), bladder cancer (FIG. 6C) and bone marrow cancer (FIG. 6D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CCTs showed thinner strands morphology corresponding to more degraded CCTs (CTS, orange arrows). Scale bar: 10 μm.

FIGS. 7A-7D depict representative immunohistochemistry (IHC) fluorescence staining images of clinical brain cancer (FIG. 7A), cervix cancer (FIG. 7B), colon cancer (FIG. 7C) and endocrine cancer (FIG. 7D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CCTs showed thinner strands morphology corresponding to more degraded CCTs (CTS, orange arrows). Scale bar: 10 μm.

FIGS. 8A-8D depict representative immunohistochemistry (IHC) fluorescence staining images of clinical esophagus cancer (FIG. 8A), head/neck cancer (FIG. 8B), heart cancer (FIG. 8C) and intestine cancer (FIG. 8D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CCTs showed thinner strands morphology corresponding to more degraded CCTs (CTS, orange arrows). Scale bar: 10 μm.

FIGS. 9A-9D depict representative immunohistochemistry (IHC) fluorescence staining images of clinical kidney cancer (FIG. 9A), liver cancer (FIG. 9B), lung cancer (FIG. 9C) and lymphatic tissue cancer (FIG. 9D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CCTs showed thinner strands morphology corresponding to more degraded CCTs (CTS, orange arrows). Scale bar: 10 μm.

FIGS. 10A-10D depict representative immunohistochemistry (IHC) fluorescence staining images of clinical melanoma cancer (FIG. 10A), mesothelium cancer (FIG. 10B), nasopharynx (FIG. 10C) and larynx cancer and oral cavity cancer (FIG. 10D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CCTs showed thinner strands morphology corresponding to more degraded CCTs (CTS, orange arrows). Scale bar: 10 μm.

FIGS. 11A-11D depict representative immunohistochemistry (IHC) fluorescence staining images of clinical ovary cancer (FIG. 11A), pancreas cancer (FIG. 11B), penis cancer (FIG. 11C) and bile duct cancer (FIG. 11D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CCTs showed thinner strands morphology corresponding to more degraded CCTs (CTS, orange arrows). Scale bar: 10 μm.

FIGS. 12A-12D depict representative immunohistochemistry (IHC) fluorescence staining images of clinical rectum cancer (FIG. 12A), skin cancer (FIG. 12B), soft tissue (smooth muscle) cancer (FIG. 12C) and stomach cancer (FIG. 12D) with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Many CTs showed thinner strands morphology corresponding to more degraded CCTs (CTS, orange arrows). Scale bar: 10 μm.

FIGS. 13A-13D depict representative immunohistochemistry (IHC) fluorescence staining images of clinical testis cancer (FIG. 13A), thyroid cancer (FIG. 13B), uterus cancer (FIG. 13C) and vulva cancer (FIG. 13D). Anti-PMCA2 rabbit polyclonal antibody showed abundant expression of PMCA2 in cytocapsular tubes (CCT, white arrows). Some CCTs showed thinner strands morphology corresponding to more degraded CCTs (CTS, orange arrows). Scale bar: 10 μm.

FIG. 14 depicts a representative immunohistochemistry (IHC) fluorescence staining image of clinical paracancer tissue of breast cancer with anti-PMCA2 rabbit polyclonal antibody anti-γ-actin antibodies and DAPI (nucleus). Anti-PMCA2 rabbit polyclonal antibody showed abundant expression of PMCA2 in cytocapsular tubes (CCT, white arrows). Scale bar: 10 μm.

FIG. 15 depicts a representative immunohistochemistry (IHC) fluorescence staining image of clinical metastatic stomach cancer migrating via CCT into lymph nodes with anti-PMCA2 rabbit polyclonal antibody anti-γ-actin antibodies and DAPI (nucleus). Many cytocapsular tubes (CCT, white arrows) were observed to wrap and invade into the lymph nodes. Scale bar: 10 μm.

FIG. 16 depicts a representative immunohistochemistry (IHC) fluorescence staining image of clinical metastatic colon cancer migrating via CCT into liver with anti-PMCA2 rabbit polyclonal antibody anti-γ-actin antibodies and DAPI (nucleus). Many cytocapsular tubes (CCT, white arrows) were observed to entangle together forming CCT masses that invade into the liver. As a result of CCT masses invasion, the liver tissues were largely disappeared. Scale bar: 10 μm.

FIG. 17 depicts a representative immunohistochemistry (IHC) fluorescence staining image of clinical metastatic adenocarcinoma originating from ovary and migrating via CCT into epiploon with anti-PMCA2 rabbit polyclonal antibody anti-γ-actin antibodies and DAPI (nucleus). Many curved cytocapsular tubes (CCT, white arrows) were observed and form bundles. Scale bar: 10 μm.

FIG. 18 depicts a representative immunohistochemistry (IHC) fluorescence staining image of clinical metastatic mucinous adenocarcinoma originating from an unknown site and migrating via CCT into epiploon with anti-PMCA2 rabbit polyclonal antibody anti-γ-actin antibodies and DAPI (nucleus). Many cytocapsular tubes (CCT, white arrows) were observed that surround and fill the space between cell mass islands within epiploon. Scale bar: 10 μm.

FIG. 19 depicts a representative immunohistochemistry (IHC) fluorescence staining image of clinical metastatic rectum cancer migrating via CCT into lymph nodes with anti-PMCA2 rabbit polyclonal antibody anti-γ-actin antibodies and DAPI (nucleus). Many cytocapsular tubes (CCT, white arrows) were observed that form curved bunches in lymph nodes. Scale bar: 10 μm.

FIGS. 20A-20E depict representative immunohistochemistry (IHC) fluorescence staining images of clinical needle biopsy of breast cancer. In the long and thin clinical tissue samples of malignant breast tumor tissues by needle biopsy, fluorescence immunohistochemistry staining assays were performed with anti-PMCA2 rabbit polyclonal antibody anti-γ-actin antibodies and DAPI (nucleus). Fluorescence images were taken by high quality fluorescence microscopes. There was no cytocapsular tube detected in site 1 or site 2, many curved and entangled cytocapsular tubes were detected (CCT, white arrows) in site 3, and many thinner e.g., silk-like strands of cytocapsular tubes corresponding to cytocapsular tubes in the process of degradation were detected (CTS, orange arrows) in sites 4 and 5. In site 3, many breast cancer cells (blue nuclei) were observed to be migrating in CCTs. Scale bar, 10 μm.

FIGS. 21A-21C depict immunohistochemistry (IHC) fluorescence staining images of clinical malignant breast cancer with rabbit polyclonal antibodies of anti-PMCA1 (FIG. 21A), anti-PMCA3 (FIG. 21B) and anti-PMCA4 (FIG. 21C). Each antibody recognizes its corresponding target protein and shows cytocapsular tubes staining (CCT, white arrows) in clinical malignant breast cancer tissues. Scale bar: 10 μm.

FIGS. 22A-22F depict schematic and microscope images showing the lifecycle of cytocapsular oncocells in vitro and in human tissues in vivo. FIG. 22A depicts a schematic diagram of cancer cell cytocapsular proteome analysis and cytocapsula (CC) and cytocapsular tube (CCT) molecular marker identification. FIG. 22B depicts representative bright field (BF) and immunohisto-chemistry (IHC) fluorescence microscope images of pancreas cancer Bxpc3 cells with cytocapsulas (CC) in the 3D CC/CCT culture kit matrix. Cancer cells with CC (white arrows), ecellulated cytocapsulas (ECC/EC, orange arrows), and cancer cell proliferation in CC (purple arrows) are shown. FIG. 22C depicts a representative IHC fluorescence microscope image of primary breast carcinoma with cytocapsular oncocells (white arrows). Incytocapsular oncocells (red arrows), ecellulated cytocapsulas (ECC, purple arrows), cytocapsular membrane (CCM, orange arrows), and cytocapsular oncocell proliferation in CC (red asterisk), and folded CC (white asterisk) are shown. FIG. 22D depicts a schematic diagram of a cytocapsular oncocell with its cytocapsula, cytocapsulasomes and nano-protrusions. FIG. 22E depicts a representative fluorescence microscope image showing that, during CCT regeneration initiation in acytocapsular oncocell mass-CC/CCT complex (AMCC) phase, a single breast cancerous cell generates multiple CCTs pointing in different directions during cancer cell's metastasis in vivo. The 7 CCTs (numbered individually) and multiple sectioned CCT fragments (SCF, purple arrows) are interconnected at the CCT node (yellow arrow) and exhibit a radical morphology. FIG. 22F depicts a representative fluorescence microscope image of a single pancreas cancer cell that generates a long, highly curved CCT in primary pancreas carcinoma tissues. Scale bar, 10 μm.

FIGS. 23A-23D depict schematic and microscope images showing the detection and lifecycle of cytocapsular oncocell. FIG. 23A depicts representative bright filed (BF) and immunohistochemistry (IHC) microscope images of cytocapsular tube (CCT) in 3D CC/CCT culture kit matrix in vitro. There is a single Bxpc3 cancer cell in migration in the CCT. CCT membrane edges (orange arrows) are shown. FIG. 23B depicts representative immunohistochemistry microscope image of cytocapsular tumorsphere (CT, purple arrows) in 3D CC/CCT culture kit matrix in vitro. Cytocapsula (CC, white arrow), ecellulated CC (ECC, orange arrows) and ECC with open holes (white asterisk) are shown. FIG. 23C depicts representative immunohistochemistry microscope image of human normal, benign and cancer tissues with CC/CCT detection by anti-PMCA2 antibodies and anti-γ-actin antibodies. Terminal duct (TD), cytocapsular tube (CCT, white arrows), CCT strand (CTS, orange arrows) are shown. FIG. 23D depicts a schematic diagram of the lifecycle of cytocapsular oncocell: transformed (cancerous) cells experience CC generation and perform cytocapsulasome-driven cytocapsula formation. Cytocapsulas wrap oncocells inside and form cytocapsular oncocells. (1) Cytocapsular oncocells proliferate and grow into cytocapsular tumors. (2) Cytocapsular oncocells proliferate and cytocapsulas elongate and develop into CCTs. (3) Ecellulation of cytocapsular oncocells produces acellular cytocapsulas and acytocapsular oncocells. Acellular cytocapsulas and CCTs perform autodegradation and decomposition and disappearance. Scale bar, 10 μm.

FIGS. 24A-24D depict microscope images showing the detection of CCTs by IHC fluorescence staining with anti-PMCA2 antibodies. FIG. 24A and FIG. 24B depict representative image of H&E staining (FIG. 24A) and IHC fluorescence staining with anti-PMCA2 antibodies (FIG. 24B) of two continuously sectioned and neighboring colon carcinoma tissue specimens. Panel 1 is the microscope image of a whole colon carcinoma core. The framed area in panel 1 is enlarged and shown in panel 2. The framed area in panel 2 is enlarged and shown in panel 3. FIG. 24C and FIG. 24D depict representative image of H&E staining (FIG. 24C) and IHC fluorescence staining with anti-PMCA2 antibodies (FIG. 24D) of two continuously sectioned and neighboring lung carcinoma tissue specimens. CCTs in colon and lung carcinoma tissues are invisible in H&E technologies (FIG. 24A and FIG. 24C), but are clearly shown in images with IHC fluorescence staining with anti-PMCA2 antibodies (FIG. 24B and FIG. 24D). Cytocapsular tube (CCT, white arrows), and CCT strand (CTS, orange arrows). Scale bar, 10 μm.

FIGS. 25A-25C depict schematic and microscope images showing the detection of CCTs, CCT formation and cytocapsular tumor lifecycle. FIG. 25A depicts representative image of H&E staining (panel 1), IHC staining with antibodies recognizing colon cancer molecular markers MSH-2 (panel 2), and IHC staining with anti-PMCA2 antibodies (panel 3) of 3 pieces of continuously sectioned colon cancer tissue specimens. The large quantities of CCTs (white arrows) presented in panel 3 are undetectable in panels 1 and 2. FIG. 25B depicts representative bright field (BF, panel 1) and fluorescence microscope (panel 2) images of the same imaged area showing that a long, straight, and stretched CCT (white arrows) links two CCs of two cytocapsular tumorspheres (CTs) with open ends in both connection sites. Cytocapsular tube (CCT, white arrows), cytocapsulas (CC1 and CC2) of the two CTs, and open end (cyan arrows) of CCT are shown. Panel 3 show a schematic diagram of two cytocapsulas interconnect by a CCT with two end opens. FIG. 25C depicts a schematic diagram of lifecycle of cytocapsular tumor: (1) Generation of cytocapsular oncocells; (2) Cytocapsular oncocells proliferate and grow into prophase cytocapsular tumor (PCT) without CCTs. Incytocapsular oncocells may experience CC generation and engender independent CCs in the PCT cytocapsular lumen. (3) PCT develops into CT with CCTs. CCTs provide physical membrane-enclosed freeways for incytocapsular oncocell metastasis. (4) CCs of cytocapsular tumors and CCTs degrade, and form acytocapsular oncocell masses of NT. (5) Some acytocapsular oncocells experience CC generation in the stressful microenvironments in NTs, and generate cytocapsular oncocells, which engender CCTs, and form Acytocapsular oncocell Mass-CT/CCT Complex (AMCC). Acytocapsular oncocells around CCTs invade into CCTs via alloentry and proceed cancer metastasis. Scale bar, 10 μm.

FIGS. 26A-I depict schematic and microscope images showing the lifecycle of cytocapsular tumor in human tissues and in vitro 3D matrix culture. FIG. 26A depicts representative bright field (BF) and fluorescence microscope images of cytocapsular tumorspheres of Bxpc3 pancreas cancer cells in 3D CC/CCT matrix culture kit. Cytocapsula (white arrow) and cytocapsular membrane (orange arrows) are shown. FIG. 26B depicts representative IHC fluorescence microscope image of initiation-stage prophase cytocapsular tumor (PCT, <20 μm in diameter/width) in early breast carcinoma. FIG. 26C and FIG. 26C depict representative IHC fluorescence microscope images of PCTs in early-development (FIG. 26C, <30 μm in diameter/width) and middle-development stage (FIG. 26D, (<40 μm in diameter/width) stages. Cytocapsula (CC, white arrows), ecellulated CC (ECC, purple arrows) are shown. FIG. 26E depicts representative IHC fluorescence microscope images of early stage cytocapsular tumor (CT) with cytocapsular tube (CCT, orange arrow) extended from CTs. FIG. 26F depicts representative IHC fluorescence microscope image of breast carcinoma tissues with high PCT and CT density. Multiple CTs merge into a bigger CT. A CT with 3 CT branches (CTBs), cytocapsula (CC, white arrow) and cytocapsular tube (CCT, orange arrow) are shown. FIG. 26G depicts representative IHC fluorescence microscope image of developed cytocapsular tumors (CTs) with thick CCT layers wrapping the CTs outside. Cytocapsula (CC, white arrow) of CTs, cytocapsular membrane (CCM, red arrow), cytocapsular tube (CCT, purple arrows), incytocapsular oncocells (cyan arrows), and CCT strand (CTS, orange arrows) are shown. FIG. 26H depicts a schematic diagram of a cytocapsular tumor with its CCTs, nano-protrusion, and incytocapsular oncocells with/without their independent CCs. FIG. 26I depicts a representative image of an acytocapsular oncocell mass-CC/CCT complex (AMCC). Nuperphase tumor (NT), CCT (orange arrow) and degraded CCT strands (CTS, pink arrow) are shown. Scale bar, 10 μm.

FIGS. 27A-27M depict distributions of cytocapsular tube tumors in 35 kinds of human normal, benign tumor, and cancerous tissues. FIG. 27A is a table of statistical CCT presence in the examined 35 kinds of human tissues and organs. FIGS. 27B-27M depict representative IHC fluorescence microscope images of CCTs (white arrows) in 35 kinds of human tissues and organs. Degraded CCT strands (CTS, orange arrows) are shown. Scale bar, 10 μm.

FIGS. 28A-28B depict schematic and microscope images showing that cytocapsulasomes drive cytocapsula growth. FIG. 28A depict real-time analysis of cytocapsula growth driven by cytocapsulasomes with a bright field phase contrast microscope. The images are taken from recorded video. Cytocapsulasome activity procedures: (1) cytocapsulasome (CS) are generated and released by oncocells, and attach to the outside cytoplasm membrane (cyan arrows); (2) CSs spontaneously are detached from oncocell surface (panels 5 and 6, orange arrows); (3) detached CSs randomly move in the cytocapsular lumen fluids (yellow arrows in panel 4, 8, 9, 10, 11); (4) CSs reach the inner side of cytocapsular membrane of CT, and contact and integrate into cytocapsular (CC) membrane of CT, and increase CC membrane size in area (red arrows, panels 8 and 9). In cytocapsular tumors with cytocapsula tightly wrapped oncocell mass surfaces, the contact and integration of cytocapsulasomes into CC membranes will be faster and more efficient without random and long movement journey in the cytocapsular lumen fluids. Incytocapsular tumorsphere (ICT), cytocapsula (CC, white arrows), cytocapsular membrane (CCM, green arrow), cytocapsular spike (purple arrows), and cytocapsulasome (CS; yellow arrow, CS in random movement in cytocapsular lumen fluid; orange arrow, CS detaching from oncocell surface; and red arrows, CS reach and fuse into CC membrane; cyan arrow: CS attached on oncocell surface) are shown. FIG. 28B depicts a schematic diagram of a cytocapsula tumorsphere with many cytocapsulasomes and spike-like structures in lumen, and cytocapsulasomes are detached from the oncocell surface and move to and fuse into the enlarged CC supporting CC growth. Scale bar, 10 μm.

FIGS. 29A-29C depict schematic, graph and microscope images showing large quantities of cytocapsulasomes promoting cytocapsula growth. FIG. 29A depicts real-time analysis of cytocapsula growth driven by cytocapsulasomes with a bright field phase contrast microscope. The images are taken from recorded video. Incytocapsular tumorsphere (ICT), cytocapsula (CC, black arrows), cytocapsula membrane (CCM, orange arrow), cytocapsulasome (CS, red and green arrows), ecellulated cytocapsula (ECC), and cytocapsular tube (CCT, in the enlarged CC lumen) are shown. FIG. 29B depicts a graph showing the quantitation of cytocapsulasomes in cytocapsular tumorspheres. FIG. 29C depicts a schematic diagram of a cytocapsular tumorsphere generating a large CC with many cytocapsulasomes in the lumen. The incytocapsular oncocell mass engender a secondary, big and “L”-shaped CCT in the big CC lumen. There are two middle acellular CCs in the big CC lumen (ECC). Scale bar, 10 μm.

FIGS. 30A-30D depict schematic, graph, table and microscope images showing the characterization of cytocapsulasome and its lifecycle. FIG. 30A depicts a representative image of cytocapsulasomes (CSs, white arrows) in CC initiation gastric cancer cells during the generation of cytocapsular oncocells in vivo. There are many CSs in the cytoplasm. FIG. 30B depicts a graph showing the quantitation of cytocapsulasomes in gastric and breast cancer cells with cytocapsulasomes in vivo. FIG. 30C depicts a table showing the comparative differences and similarities between cytocapsulasome and other 4 extracellular vesicles. FIG. 30D depicts a schematic diagram of cytocapsulasome lifecycle: (1) Generation of CS in the cytoplasm of CC initiation oncocells, (2) Release of CS onto the outside of cytoplasm membrane, and attach to the cell membrane surface, (3) Multiple CSs on the cell membrane contact and integrate into cytocapsular membrane fragments, (4) Cytocapsular membrane fragment grow up with more CS integration, (5) Cytocapsular membranes envelope the whole single cell and generate cytocapsular oncocells, and isolate them from the ECM, (6) CSs reach and integrate into cytocapsular membranes and increase cytocapsular membrane areas, (7) Cytocapsulas grow up and generate enlarged cytocapsulas or elongate and develop into cytocapsular tubes. Scale bar, 10 μm.

FIGS. 31A-31I depict schematic, graph, and microscope images showing cytocapsular tube generation and elongation, and cell migration in CCTs in vitro and in vivo. FIG. 31A depict real-time analysis of cytocapsular tube initiation, generation and elongation in vitro in 3D matrix CC/CCT culture kit using MCF-7 cell. The images are taken from recorded video. Cytocapsular tube (CCT, red arrows), cell in CCT (white arrows), bleb (cyan arrows), cytocapsular spike (CSP, yellow arrows) are shown. FIG. 31B depicts a graph showing the quantitation of cytocapsular tube elongation speed in the CC/CCT culture kit matrix in vitro. FIG. 31C depicts a representative image of initial cytocapsular tube (IC, white arrows) in breast cancer tissues in vivo. FIG. 31D depicts a schematic diagram of cytocapsular tube elongation: (1) Incytocapsular oncocell generate and release cytocapsulasomes and drive cytocapsular membrane area increase, (2) cytocapsular oncocells generate many blebs in all directions, sense microenvironments, choose and decide the motility directions, (3) cytocapsulasomes continuously drive cytocapsulas (CC) membrane increase in areas with many blebs in the CC lumen, (4) cytocapsular oncocells move forward, and elongate CCT length, and generate long CCTs. FIG. 31E depicts a representative image of cell migration in CCT in vitro. FIG. 31F depicts real-time analysis of incytocapsular oncocell migration in CCTs. The images are taken from recorded video. Cytocapsular tube (CCT, red arrows), incytocapsular oncocell in migration (white arrows), migration direction (pink arrows), reversed migration direction (yellow arrows), cell with lamellipodia at the leading edge (red asterisk), cell in transition of migration direction in CCT (blue asterisk) are shown. FIG. 31G depicts a graph showing the quantitation of Bxpc3 pancreas cancer cell migration in CCT in vitro. FIG. 31H depicts a representative image of colon cancer cells in migration in colon CCTs. Colon cancer incytocapsular oncocells in CCTs are in thin, long and spindle-shaped morphologies. Cytocapsular tube (CCT, red arrows), and colon oncocells in migration in CCT (white arrows) are shown. FIG. 31I depicts a schematic diagram of cytocapsular tube tumor. Scale bar, 10 μm.

FIGS. 32A-32C depict microscope images showing integrated cytocapsular oncocell, cytocapsular tumorspheres, CCT networks, and cytocapsular tumorsphere network systems in vitro. FIG. 32A depicts representative images of primary and secondary cytocapsular tumorspheres integrated cytocapsular tumorsphere-network systems (CTNSs) in vitro. Firstly, primary cytocapsular tumorspheres (black asterisks) interconnect by CCT networks. Disseminated incytocapsular oncocells gather in the CCT network nodes and grow into secondary cytocapsular tumorspheres (red asterisks). Secondary cytocapsular tumorspheres interconnect primary cytocapsular tumorspheres via CCTs and generate a combined primary and secondary cytocapsular tumorsphere network system in the CC/CCT culture kit (6-well plate). FIG. 32B depicts representative images of cytocapsular tumorsphere interconnection by CCTs with open-ends at both sides of CCT, and incytocapsular oncocell migration in CCTs and CTNSs. FIG. 32C depicts representative images of membrane-sheltered cytocapsular oncocells, cytocapsular tumorspheres, CCT networks, and integrated CTNSs. After ecellulation, the acellular CCs and CCTs show the interconnected CC and CCT membrane systems of integrated and interconnected primary (white asterisks) and secondary (red asterisks) CT network systems (CTNSs). Cytocapsular tube (CCT, white arrow), cytocapsula (CC, yellow arrows), open-end of both sides of CCTs (cyan arrows), and incytocapsular oncocell migration in CCTs (red arrows) are shown. Scale bar, 10 μm.

FIGS. 33A-33L depict microscope images showing the lifecycle of primary cytocapsular tumor network systems in vivo. FIG. 33A depicts a representative image of dense prophase cytocapsular tumor (PCT, white arrows) groups in primary invasive ductal breast carcinoma. Degradation of acellular cytocapsula (CC) leads to cloud-like CC strand masses (orange arrows). Framed areas 1 and 2 are enlarged and shown in FIGS. 34A and 34B. FIG. 33B depicts a representative image of primary invasive ductal breast carcinoma with dense cytocapsular tumors (CTs) and CT network systems (CTNSs). The white dashed line framed area is enlarged and shown in FIG. 33C. FIG. 33C depicts the enlarged framed area of FIG. 33B. CTs, cytocapsula (CC, purple arrows), cytocapsular membrane (CCM, orange arrows), CCTs (orange arrows) and degraded CCT strands (CTSs, pink arrows) are shown. FIG. 33D depicts a representative image of a CT is wrapped by thick CCT layer outside. CCT outside CC (white arrows), CCT inside CC (red arrows), CC (purple arrows), and CC membrane (CCM, orange arrows) are shown. FIG. 33E depicts a representative image of CCT (white arrows) networks in primary CTNSs in primary colon cancer. FIG. 33F depicts a representative image of an early nuperphase tumor. The CT cytocapsula is degraded with CC fragments (orange arrow) remain. CCTs degrade into CCT strands (CTSs, white arrows). FIG. 33G depicts a representative image of a late stage of nuperphase tumor. There are dense acytocapsular oncocells (AO, cyan arrows) without CCTs. FIG. 33H depicts a representative image of acytocapsular oncocell mass-CC/CCT complex (AMCC) in primary breast cancer. Many new CCTs (white arrows) are regenerated by some acytocapsular oncocells. FIG. 33I depicts a representative image of CCT degradation in AMCC. Acytocapsular oncocells (AO, cyan arrows) and CCT (white arrows) are shown. FIG. 33J depicts a representative image showing that many acytocapsular oncocells invade into CCTs (white arrows) via alloentry, metastasize and leave away. The local oncocell density is very low. Many CCTs are in degradation into CCT strands (CTS, orange arrow). FIG. 33K depicts a representative image showing severe CCT degradation with many CCT fragments and strands (CTS, orange arrow). FIG. 33L depicts a representative image showing CCT degradation and oncocell apoptosis in cytocapsular tumor lumens. Cytocapsula (CC, purple arrows), CCT (yellow arrows), cytocapsular tube strand (CTS, orange arrows), apoptotic oncocells in CC (cyan arrows) are shown. Normal tissue cell apoptosis, acytocapsular oncocell metastasis via CCTs or apoptosis, and CCT degradation caused cavities (black areas, tissue liquefaction, orange asterisks) in FIGS. 33J-33L are shown. Scale bar, 10 μm.

FIGS. 34A-34F depict microscope images and graphs showing prophase CT, CT and CCT networks in vivo. FIG. 34A depict the enlarged image area 1 from FIG. 33A. Early prophase cytocapsular tumors (EPCTs, size <50 μm and incytocapsular oncocell number <20; white arrows) are shown. FIG. 34B depicts a representative image showing dense PCT and ecellulated cytocapsulas (ECC, purple arrows) in the enlarged CC lumen are shown. FIG. 34C depicts a graph showing the quantitation of PCT density in breast, colon and prostate cancers. FIG. 34D depicts a graph showing the quantitation of CT density in breast, colon and prostate cancers. FIG. 34E depicts a representative image showing CCT networks composed by straight CCTs in primary breast cancer. FIG. 34F depicts a representative image showing CCT networks composed by curled and coiled CCTs in primary breast cancer. Scale bar, 10 μm.

FIGS. 35A-35D depict microscope images and table showing that CCTs are broadly present in native tissue adjacent to tumors (NAT). FIG. 35A depicts a representative image showing NAT in primary breast carcinoma with large quantities of CCT networks in high density. FIG. 35B depicts a representative image showing NAT in bone marrow of primary plasma cell myeloma with CCT (white arrows) bunches coexist with immune cells. FIG. 35C depicts a representative image showing NAT of trabecular bone in primary plasma cell myeloma. CCTs (white arrows) invade into hard tissues of trabecular bone. There is no PMCA2 signal in bone matrices. FIG. 35D depicts a table showing the quantitation of CCT density in NAT of 14 kinds of human cancer tissues. Scale bar, 10 μm.

FIGS. 36A-36J depict microscope images showing cytocapsular oncocell metastasis in primary CTNSs in primary cancer niche. FIG. 36A depicts a representative image of CCTs in loose soft tissue in lung cancer. FIG. 36B and FIG. 36C depict representative images of CCTs in compact soft tissues in thyroid (FIG. 36B) and oral (FIG. 36C) cancers. FIG. 36D depicts a representative image of CCT superstructures in pancreas cancer. FIG. 36E depicts a representative image of massive curled CCT networks invade into compact acytocapsular oncocell masses and form AMCC. FIG. 36F depicts a representative image showing dense CCT network masses invading through compact primary prostate cancer tissues in AMCC. FIG. 36G depicts a representative image of CCT bunches in primary breast carcinoma CTNSs. FIG. 36H depicts a representative image of CCT masses in primary breast carcinoma CTNSs and most acytocapsular oncocells left via CCTs. (I) Representative image of highly curled and coiled CCTs in primary CTNSs in primary pancreas cancer with many acytocapsular oncocells left via CCTs. FIG. 36J depicts a representative image of complex CCT superstructures in primary CTNSs. CCT (white arrows) and CCT strand (CTS, orange arrows) are shown in FIGS. 36A-36J. Scale bar, 10 μm.

FIGS. 37A-37F depict schematic, table and microscope images showing that cytocapsular tube networks dominate cancer metastasis in vivo. FIG. 37A depicts a representative image of CCTs and blood vessel in primary prostate cancer tissues. CCTs (white arrows) and incytocapsular oncocell in migration in CCTs (purple arrows) are shown. FIG. 37B depicts a table showing the quantitative comparison of cytocapsular tubes and humoral vessels in primary cancer niche, normal tissue adjacent to the tumor (NAT), and secondary cancer niche. FIGS. 37C and 37D depict representative images of micro blood vessels wrapped by CCTs, CCT invasion into blood vessels, and release of incytocapsular oncocells into the blood as resources of circulating tumor cells (CTCs). Framed area in FIG. 37C is enlarged and shown in FIG. 37D. FIG. 37D depicts the enlarged area from FIG. 37C. The incytocapsular oncocell (ICO, yellow arrow) entering blood, CCT (white arrows), CCT strand (CTS, orange arrow) and red blood cell (RBS, purple arrows) are shown. FIG. 37E depicts a representative image of late cancer stage with many red blood cells distributed in tissues caused by CCT invasion-damaged blood vessels. CCT (white arrows), CCT strand (CTS, orange arrow) and red blood cell (RBS, purple arrows) are shown. FIG. 37F depicts a schematic diagram of CCT invasion, CTC resource, micro blood vessel damage, and red blood cell distribution in tissues beyond blood vessels. Scale bar, 10 μm.

FIGS. 38A-38B depict microscope images showing that large quantities of cytocapsular tube networks beyond humoral vessels dominate cancer metastasis pathways. FIG. 38A depicts a representative image of clinical prostate cancer at Stage IIb with large quantities of CCTs and CCT networks occupying most of the spaces beyond humoral vessels (blood vessel, lymph vessels) and prostate grand lumens, and dominate cancer cell metastasis pathways. Most of prostate cancer cells have left away via CCT networks. The white dashed framed area is enlarged in FIG. 38B. Micro blood vessels (MBV, orange arrows) are shown. FIG. 38B depicts the enlarged image in framed area in FIG. 38A. There are large quantities of CCTs (white arrows) and CCT networks beyond the integrated micro blood vessels (MBV). Most of prostate cancer cells have left away via CCT networks. MBVs are intact without damage at this cancer stage. Degraded CCT strands (CTS, red arrow) are shown.

FIGS. 39A-39L depict microscope images showing the lifecycle of secondary cytocapsular tumor network systems in vivo. FIG. 39A depicts representative images of metastatic breast cancer CCT (white arrows) thin (1) and thick (2) bunches wrapping and invading into lymph nodes. FIG. 39B depicts a representative image of metastatic cervix squamous cell carcinoma 3D CCT (white arrows) networks invade into and distributed inside most lymph node areas. White dashed line framed areas 1, 2 and 3 are enlarged in FIG. 40A, FIG. 39C, and FIG. 40B. FIG. 39C depicts the enlarged white dashed line framed area 2 in FIG. 39B. Metastatic cervix squamous cell carcinoma CCTs invade into lymph node, develop into CCT masses and occupy spaces in lymph nodes. Cross-sectioned CCTs (orange arrows) are shown. FIG. 39D depicts metastatic breast CCTs invaded into lymph nodes grow into multiple cytocapsular tumors (CTs). Enlarged cytocapsula (CC, purple arrows), CC membrane (CCM, yellow arrows), and CT lumens (white asterisks) are shown. FIG. 39E depicts a representative image of secondary breast CTNSs with high CT density in lymph node. Breast CCTs invade into lymph node, incytocapsular oncocells proliferate and grow into many cytocapsular tumors. Large quantities of secondary breast cytocapsular tumors in lymph node interconnect by CCT networks and form dense CTNSs in lymph node. Cytocapsular tumor (CT, yellow arrows), cytocapsula (CC, white arrows), CCT (orange arrows), CCT strand (CTS, purple arrow), and cytocapsular lumens (white asterisks) are shown. FIG. 39F depicts a representative image showing degradation of CCs of secondary CTs, CCT degradation, and acytocapsular ovary oncocell uncontrolled proliferation led to enlarged acytocapsular ovary oncocell masses in secondary CTNSs in omentum. FIG. 39G depicts that some acytocapsular ovary oncocells regenerate CCTs, and form AMCC in secondary CTNSs in omentum. CCT (white arrows), acytocapsular oncocell (AO, cyan arrows), and oncocells in migration in CCTs (purple arrows) are shown. FIG. 39H depicts a representative image of secondary cervix CTNSs in lymph node with many cervix CCTs in lymph node. CCT (white arrows) and oncocells in migration in CCTs (purple arrows) are shown. FIG. 39I depicts a representative image of a whole tissue core of secondary rectum CTNSs in mesentery with high CCT (white arrows) density. White framed areas 1, 2 and 3 are enlarged and shown in FIG. 40E, FIG. 39J and FIG. 40F. FIG. 39J depicts the enlarged area from FIG. 39I to show the high CCT density. CCT (white arrows) and oncocells in migration in CCTs (purple arrows) are shown. FIG. 39K depicts a representative image showing secondary colon CTNSs with severe CCT degradation in liver. CCT (white arrows) and oncocells in migration in CCTs (purple arrows) are shown. FIG. 39L depicts a representative image showing secondary hepatocellular carcinoma CTNSs with severe CCT degradation, severe blood vessel damage, and severe red blood cell leak, major acytocapsular oncocell metastasis via CCTs (white arrows), and low cell density in the local cerebrum. Red blood cell (RBC, red arrows) randomly distributed outside blood vessels and in tissues, and CCT strand (CTS, orange arrows) are shown. Normal tissue cell apoptosis, acytocapsular oncocell metastasis via CCTs or apoptosis, and CCT degradation caused cavities (black spaces, tissue liquefaction, orange asterisks) in FIGS. 39K-39L are shown. Scale bar, 10 μm.

FIGS. 40A-40F depict microscope images and graph showing metastatic CCTs, secondary CT density and secondary AMCC in vivo. FIG. 40A depicts the enlarged image from white dashed line framed area 1 in FIG. 39B. The 3D metastatic cervix squamous cell carcinoma CCTs (white arrows) invade into lymph nodes and form highly curved/coiled CCT bunches and masses and coexist with immune cells in lymph nodes. FIG. 40B depicts the enlarged image from white dashed line framed area 3 in FIG. 39B. The 3D metastatic cervix squamous cell carcinoma CCTs (white arrows) randomly invade into lymph nodes, coexist with immune cells in lymph nodes, and occupy spaces in lymph nodes. FIG. 40C depicts a graph showing the quantitation analysis of secondary breast CTs in bladder, liver and lymph node. FIG. 40D depicts a representative image of degradation of CCs of secondary CTs, CCT degradation, and acytocapsular ovary oncocell uncontrolled proliferation led to enlarged acytocapsular ovary oncocell masses in secondary CTNSs in omentum. Acytocapsular oncocells (AO, cyan arrows) masses are shown. FIG. 40E depicts the enlarged image from white dashed line framed area 1 in FIG. 39I. There are very dense and massive CCT network bunches in secondary CTNSs of rectum cancer in mesentery. FIG. 40F depicts the enlarged image from white dashed line framed area 3 in FIG. 39I. There are very dense CCT network masses in secondary CTNSs of rectum cancer in mesentery.

FIG. 41 depicts a schematic diagram of an atlas of cytocapsular oncocell evolution lifecycle in vivo. A simplified atlas of cytocapsular oncocell evolution lifecycle in vivo includes 10 steps: (1) Normal cell transformation generates abnormal and acytocapsular oncocell caused by accumulated gene mutation and chemical and physical stimuli from extracellular microenvironments. (2) By unknown molecular mechanisms, some acytocapsular oncocells experience CC generation: generation of cytocapsulasomes and cytocapsulas enclosing the cell and isolating the cell from stressful microenvironments. The additional extracellular protective cytocapsula of cytocapsular oncocell advance to survive under stressful microenvironments. (3) Incytocapsular oncocell proliferate in cytocapsular lumen and generate prophase cytocapsular tumor (PCT) and cytocapsular tumor (CT). (4) CTs in the primary niche generate CCTs, CCT networks, and acytocapsular oncocell mass-CC/CCT complex (AMCC). (5) All CTs in primary niches interconnect by CCT networks and form primary cytocapsular tumor network systems (CTNSs). (6) Primary CCT networks expand and invade into neighboring and far distance tissues and organs. (7) CCT branching morphogenesis, new CCT network formation, and new CT formation and growth in secondary niches. (8) All CTs in the secondary niche interconnect with CCT networks and form secondary CTNSs. AMCC formation in secondary niches. (9) Primary and secondary CTNSs have existed interconnections with CCT metastatic CCT networks, and form integrated primary and secondary CTNSs. (10) A series of activities and responses of CTs, CCTs, CCT networks and CTNSs under various conditions shape the dynamic integrated CTNSs. The expansion and invasion of CCTs, CTs, CTNSs and AMCCs in normal tissues and organs lead to normal cell apoptosis, normal tissue biological function failure and structure damage. Normal cell apoptosis, acytocapsular oncocell apoptosis or leave-away via CCT networks, and CCT degradation result in local cavities filled with intercellular fluids but without tissues (named tissue liquefaction) and tissue/organ biological function failure.

### DETAILED DESCRIPTION

Embodiments of the present disclosure are based on the surprising discovery that endogenous plasma membrane Ca²⁺-ATPases (PMCAs) are highly expressed in the membrane of cytocapsular tubes (CCT) in cancer tissues. Early research has reported that single human cells under optimal 3D environments generated novel facultative membranous organelles of cytocapsulae and cytocapsular tubes (Tingfang Yi and Gerhard Wagner, Cytocapsular Tubes Conduct Cell Translocation. Proc Natl Acad Sci USA. 2018 February 6; 115(6):E1137-E1146). The report showed that cytocapsular tubes conducted cell migration and translocation in vitro and in tissues in vivo in xenografted mouse model. However, cytocapsulae and cytocapsular tubes are hard to detect due to a lack of markers for these novel membranous organelles. The inventor of the present disclosure has surprisingly found that endogenous plasma membrane Ca²⁺-ATPases (PMCAs) are highly expressed in the membranes (i.e., the cytocapsular membranes) of the cytocapsulae and cytocapsular tubes in vivo in cancerous tissues. However, no PMCA expression or CCT was detected in healthy or benign tumor tissues. It was also found that the density and morphology of the cytocapsular tubes detected in the cancer tissues can be used to predict the degree of cancer progression and cancer metastasis grade, providing a novel standard for clinical cancer analyses including diagnosis, prognosis, and therapy evaluation.

Currently, cancer treatment mainly includes surgery, radiation therapy, chemotherapy, immunotherapy and hormone therapy. Early detection and accurate diagnosis is key to successful treatment. Although tumor markers have been identified for some types of cancers, in many other cancer types, no tumor marker has been found for them that are useful for cancer diagnosis. Therefore, not all cancers can be easily detected or diagnosed with a specific marker. In addition, since most of the currently known tumor markers are present in a very small amount in cancer cells or tissues, highly sensitive measurement methods and special techniques are required for detection. Embodiments of the present disclosure are directed to novel and simple methods that allow for detection of all cancers with high sensitivity and accuracy. According to some embodiments, tissues from anywhere of a subject can be obtained. According to some embodiments, the tissue can be healthy, benign or cancerous. According to other embodiments, the cancerous tissues include primary sites/niches where the tumor cells originate from or secondary sites/niches where the tumor cells spread to.

It is expected that the detection and diagnostic applications for all cancers will be opened with the availability of the novel methods described herein. In cases when the tumor is not large enough to be confirmed with the naked eye, be found by ultrasonic examination, computerized tomography scan or MRI (nuclear magnetic resonance imaging), these hard to find “invisible” tumors by conventional means can be detected by the methods described herein. Since an invisible part of cancer can be diagnosed by the present methods, the present methods are useful for early detection of cancer metastasis in situations when the tumor is small but highly malignant.

A characteristic of cancer is the ability of cancer cells to metastasize. Embodiments of the present disclosure are directed to detecting cancer tissues in primary site of occurrence. Embodiments of the present disclosure are also directed to detecting secondary sites of metastasized cancer tissues. Thus, the present methods are useful to detect and diagnose cancer that has occurred in the invisible part, diagnose cancer progression, diagnose malignancy of cancer, post operation and treatment progress diagnosis, recurrence diagnosis, metastasis diagnosis, etc.

Cancer diagnosis involves determination of the degree of cancer progression. There are different ways to classify the degree based on the degree of spread at the primary site of the tumor and the presence or absence of metastasis to regional lymph nodes or distant organs. For example, there are clinical stage and pathological stage for cancer progression and the degree of cancer progression also depends on the grade, location, organ, tumor markers and genetics. Most cancers that involve a tumor (solid tumor cancer) as opposed to e.g., blood cancer are classified into five broad clinical stages. Other kinds, like blood cancers, lymphoma, and brain cancer, have their own staging systems. For clinical cancer stage, stage 0 means there's no cancer, only abnormal cells with the potential to become cancer. This is also called carcinoma in situ. Stage I means the cancer is small and only in one area. This is also called early-stage cancer. Stages II and III mean the cancer is larger and has grown into nearby tissues or lymph nodes. Stage IV means the cancer has spread to other parts of a subject's body. It's also called advanced or metastatic cancer. These broad groups are based on a much more detailed system that includes specific information about the tumor and how it affects the rest of the body. Another common cancer stage system is the TNM system, short for tumor, node, and metastasis. Embodiments of the present disclosure are directed to methods that provide a uniform way for detecting, predicting and determining the cancer metastasis stages based on the detection of cytocapsular tubes that is common to all cancer tissues. In one embodiment, the method includes the detection of PMCA expression in cytocapsular tubes in tissues of a subject. In some embodiments, PMCA-1, PMCA-2, PMCA-3 and PMCA-4 expression in the tissue and/or cytocapsular tubes in the tissue are detected by the method described herein.

Predicting or determining cancer metastatic stages are important for cancer treatment options. For example, an early-stage cancer may call for surgery while an advanced-stage cancer may need chemotherapy. The outlook for recovery also depends in part on how early the cancer is detected. When the degree of progression of cancer is known, in addition to determining an appropriate treatment regime, it is possible to determine the therapeutic effect of anticancer drugs. Specific examples of treatment decisions may differ depending on the type of cancer. For example, prostate cancer which usually has a very low grade of malignancy can be treated differently from bone metastasis which usually causes death and patients with pain. Treatments such as hormonal therapy and excision surgery have side effects, so it is necessary to appropriately determine the treatment method for different cancer. In addition, if it is possible to appropriately determine whether or not the selection of an anticancer agent is appropriate and the timing of finishing the administration of the anticancer agent, the physical and economic burden on the patient can be reduced. Embodiments of the present methods provide for determining cancer metastatic stages in a uniform fashion across all solid tumor cancer types, ushering a new standard in cancer detection, diagnosis, treatment and prognosis. Furthermore, according to the embodiments of the present disclosure, it is possible to diagnose the degree of cancer progression such as tumor growth, infiltration into surrounding tissues, and cancer metastasis to lymph nodes and distant organs.

Embodiments of the present disclosure are directed to detecting cytocapsular tubes in in the so called “normal adjacent tissues” (NAT) surrounding the tumor tissue. During surgical removal of a tumor, even if the tumor is small, if the malignancy is high, it usually requires a large margin at the time of tumor removal, or to observe the area with attention to a wide range of surrounding tissues. When postoperative diagnosis including recurrence and metastasis can be made, the present method can be used to detect the presence of cytocapsular tubes in surrounding tissues of tumor to diagnose whether the tumor has been completely removed by surgery and the risk of the remaining NAT for recurrence and metastasis. If the excision is not complete, recurrence is likely to occur, and the present methods of detecting CTs in surrounding tissues can be used as a basis for follow-up at shorter intervals and, in some cases, early reoperation during cancer treatment. In addition, it is highly possible that CTs can be detected early if tumor recurs by the methods described herein. If remote metastasis has occurred, the present methods provide a criterion for expanding the examination range beyond the excision site and its surroundings.

Embodiments of the present disclosure are directed to methods that allow for detecting cancer metastasis in early stages since the methods detect the cytocapsular tubes in tissues, which do not depend on the size of the tumor. Therefore, even if the tumor is invisible, if it is metastatic, it can be detected. This increases the potency and accuracy of cancer diagnosis. In addition, the present methods also make a significant contribution to regular health checkups, cancer screenings, preoperative diagnosis, and treatment option decisions.

Aspects of the present disclosure are based on the heretofore undiscovered observation that endogenous PMCAs are highly expressed in the membranes of cytocapsular tubes in cancer tissues. As observed, cytocapsular tubes are present in caner tissues but not in normal tissues or benign tissues. In the case of a benign tumor, surgery can be completed even if the tumor is large. After the operation, it is only necessary to care for the excised part, and it is not necessary to perform expensive anticancer drug treatment or follow-up. Since no PMCA expression and no cytocapsular tube is detected in benign tumor by the present methods, the present methods provide a convenient and simple cancer detection method that allow for quick screening and treatment option decisions for humans and animals.

According to certain aspect, the detection of cytocapsular tubes in cancer tissues is in vitro, in vivo or ex vivo.

In some embodiments, the expression of PMCAs is measured in a tissue sample separated, derived, or obtained from a subject. Methods of obtaining tissues for cancer detection and diagnosis are known to those skilled in the art. According to one embodiment, the tissue or tissue sample is obtained from a subject by biopsy. Methods for preparation of biopsy tissue samples including tissue collection, fixation and sectioning are well-known in the art. In some embodiments, the biopsy includes clinical biopsy, bone marrow aspiration and biopsy, cardiac biopsy, core biopsy, endometrial biopsy, endoscopic biopsy, excisional and incisional biopsy, fine-needle aspiration biopsy, lymph node biopsy, needle biopsy, open biopsy, punch biopsy, sentinel lymph node biopsy, shave biopsy, skin biopsy and the like.

According to certain embodiments, the present methods are useful for detection and diagnosis of all kinds of cancer tissues of primary origin and secondary metastasis. Tissue from anywhere in a subject's body can be obtained for detection and screening by the present methods. For example, the primary and secondary cancers that can be detected by the present methods include but are not limited to the following and their related types and subtypes: acute lymphoblastic leukemia (ALL), acute myeloid leukemia (AML), cancer in adolescents, adrenocortical carcinoma, AIDS-related cancers, Kaposi sarcoma (Soft Tissue Sarcoma), AIDS-related lymphoma (Lymphoma), primary CNS lymphoma (Lymphoma), anal cancer, appendix cancer, astrocytomas, childhood (brain cancer), atypical teratoid/rhabdoid tumor, childhood central nervous system (brain cancer), basal cell carcinoma of the skin, bile duct cancer, bladder cancer, bone cancer (includes Ewing sarcoma and osteosarcoma and malignant fibrous histiocytoma), brain tumors, breast cancer, bronchial tumors (lung cancer), Burkitt lymphoma, carcinoid tumor (gastrointestinal), carcinoma of unknown primary, childhood cardia (heart) tumors, tumors of central nervous system, medulloblastoma and other CNS embryonal tumors, childhood (brain cancer), germ cell tumor, childhood (brain cancer), primary CNS lymphoma, cervical cancer, cholangiocarcinoma, chordoma, childhood (bone cancer), chronic lymphocytic leukemia (CLL), chronic myelogenous leukemia (CML), chronic myeloproliferative neoplasms, colorectal cancer, craniopharyngioma, childhood (brain cancer), cutaneous T-cell lymphoma, ductal carcinoma in situ (DCIS), embryonal tumors, endometrial cancer (uterine cancer), ependymoma, childhood (brain cancer), esophageal cancer, esthesioneuroblastoma (head and neck cancer), extracranial germ cell tumor, childhood, extragonadal germ cell tumor, eye cancer, intraocular melanoma, retinoblastoma, fallopian tube cancer, malignant fibrous histiocytoma of bone and osteosarcoma, gallbladder cancer, gastric (stomach) cancer, gastrointestinal carcinoid tumor, gastrointestinal stromal tumors (GIST) (soft tissue sarcoma), gastrointestinal tract lymphoma, gastrointestinal lymphoma, germ cell tumors, childhood extracranial germ cell tumors, ovarian germ cell tumors, testicular cancer, gestational trophoblastic disease, hairy cell leukemia, childhood heart tumors, hepatocellular (liver) cancer, Hodgkin lymphoma, hypopharyngeal cancer (head and neck Cancer), intraocular melanoma, islet cell tumors, pancreatic neuroendocrine tumors, kidney (renal cell) cancer, langerhans cell histiocytosis, laryngeal cancer (head and neck cancer), leukemia, liver cancer, lung cancer (non-small cell, small cell, pleuropulmonary blastoma, and tracheobronchial tumor), lung or uterine adenocarcinoma, lymphoma, male breast cancer, melanoma, intraocular melanoma, merkel cell carcinoma (skin cancer), malignant mesothelioma, malignant mixed tumor, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides (lymphoma), myelodysplastic syndromes, myelodysplastic/myeloproliferative neoplasms, chronic myeloproliferative neoplasms, gingival tumor, perianal adenocarcinoma, anal cyst mass, anal sac apocrine adenocarcinoma, Sertoli cell tumor, vaginal vestibular cancer, sebaceous gland cancer, sebaceous epithelioma, sebaceous adenoma, sweat gland cancer, intranasal adenocarcinoma, nasal adenocarcinoma, colon cancer, bronchial adenocarcinoma, adenocarcinoma, ductal carcinoma, mammary carcinoma, combined breast carcinoma, malignant mixed mammary tumor, intraductal papillary adenocarcinoma, fibrosarcoma, hemangioperiosteoma, chondrosarcoma, histiocytic sarcoma, myxoid sarcoma, anaplastic sarcoma, mastocytoma, cutaneous leiomyoma, intraperitoneal leiomyoma, leiomyoma, chronic lymphocytic leukemia, lymphoma, small to medium cell lymphoma, adrenal medullary tumor, granulosa, cystoma, nasal cavity and paranasal sinus cancer, nasopharyngeal cancer, neuroblastoma, non-Hodgkin lymphoma, oral cancer, lip and oral cavity cancer and oropharyngeal cancer, ovarian cancer, pancreatic cancer, pancreatic neuroendocrine tumors (islet cell tumors), papillomatosis (childhood laryngeal), paraganglioma, parathyroid cancer, penile cancer, pharyngeal cancer, pheochromocytoma, pituitary tumor, plasma cell neoplasm/multiple myeloma, pregnancy and breast cancer, primary central nervous system (CNS) lymphoma, primary peritoneal cancer, prostate cancer, rare cancers of childhood, rectal cancer, recurrent cancer, retinoblastoma, salivary gland cancer, sarcoma, childhood rhabdomyosarcoma (soft tissue sarcoma), childhood vascular tumors (soft tissue sarcoma), soft tissue sarcoma, uterine sarcoma, Sézary syndrome (lymphoma), skin cancer, small intestine cancer, squamous cell carcinoma of the esophagus, squamous cell carcinoma of the skin, metastatic squamous neck cancer with occult primary, cutaneous T-cell lymphoma, throat cancer, hypopharyngeal cancer, thymoma and thymic carcinoma, thyroid cancer, tracheobronchial tumors (lung cancer), transitional cell cancer of the renal pelvis and ureter (kidney (renal cell) cancer), carcinoma of unknown primary, urethral cancer, endometrial uterine cancer, uterine sarcoma, vaginal cancer, vascular tumors (soft tissue sarcoma), vulvar cancer, Wilms tumor and other childhood kidney tumors, and cancer in young adults.

The plasma membrane Ca²⁺ ATPase (PMCA) is a transport protein in the plasma membrane of cells. One of the functions of PMCA is to remove calcium (Ca²⁺) from the cell, hence also named plasma membrane Ca²⁺ pumps (PMCAs). Regulation of Ca²⁺ transport is known to be vital in physiological processes, including lactation, proliferation and apoptosis. The plasma membrane Ca²⁺ pumps (PMCAs) are a family of P-type ATPases that comprise four isoforms (PMCA1-4) encoded by different genes, with additional variants generated by alternative splicing (Jason I E Bruce, Metabolic Regulation of the PMCA: Role in Cell Death and Survival. Calcium 2018: 69:28-36). The plasma membrane Ca²⁺ pump isoform 2 (PMCA2), a calcium ion efflux pump, was the first protein identified to be crucial in the transport of Ca²⁺ ions into milk during lactation in mice. In the rat and mouse mammary gland from lactating animals, PMCA2 is the predominant isoform (Peters A et, al., The Calcium Pump Plasma Membrane Ca(²⁺)-ATPase 2 (PMCA2) Regulates Breast Cancer Cell Proliferation and Sensitivity to Doxorubicin. Sci Rep 2016 May 5; 6:25505). The diverse distributions of these isoforms suggest that they play distinct roles. PMCA isoforms 1 and 4 are ubiquitously expressed, including in the brain, skeletal muscles and breast tissues, whereas PMCA2 and PMCA3 show a more restricted distribution, with PMCA2 expression found in the brain, heart and breast. Emerging evidence supports the notion that alterations in PMCA expression or activity are linked to a number of pathological conditions (Jeong J, et al., PMCA2 Regulates HER2 Protein Kinase Localization and Signaling and Promotes HER2-mediated Breast Cancer. Pro Natl Acad Sci USA 2016; 113 (3):E282-90). However, whether endogenous PMCAs (PMCA1-4) are expressed in the membranes of cytocapsular tubes in vivo or in vitro are not previously known.

According to some embodiments, endogenous expression of PMCA1-4 in the membranes (cytocapsular membranes) of cytocapsular tubes, cytocapsulars, cytocapsulasome vesicles, cytocapsular oncocells, cytocapsular tumorspheres, and cytocapsular tumorsphere network systems from cancer tissues were detected by the present methods using anti PMCA1-4 antibodies produced from rabbits against PMCA antigens. These antibodies bind to any of PMCA1, PMCA2, PMCA3, or PMCA4. According to some embodiments, the antibody that binds to any of PMCA1-4 was raised in commonly used animal species in the laboratory for polyclonal antibody production, including but not limited to rabbits, rats, mice, guinea pigs, hamsters, goats, chickens, sheep and llamas, and the like according to methods known to one skilled in the art.

Methods for measuring or detecting the expression of proteins in tissues are known to those skilled in the art. Immunological-based methods such as quantitative enzyme-linked immunosorbent assays (ELISA), Western blotting and dot blotting are very common and sensitive assays for protein detection, and they use antibodies that react specifically with entire proteins or specific epitopes (e.g., fusion tags) after tissue or cell lysis. According to some embodiments, immunoassays using anti-PMCA antibodies are used to detect and measure the expression of PMCA in the tissues of a subject. The detection methods are not limited to immunoassays. mRNAs of PMCA can also be detected by methods known to those skilled in the art. Any methods known in the art can be used to detect and measure the expression of PMCA in the membranes of the cytocapsular tubes in tissue samples from a subject. As used herein the term “measurement” includes detection, qualitative, quantitative, and semi-quantitative.

According to some embodiments, measurement of antibody binding to an antigen can be performed by immunoassay by antigen-antibody reaction known to those skilled in the art. The immunoassay method itself is a well-known conventional method to those skilled in the art. As specifically described in the Examples herein, the antibodies raised against human PMCAs can detect human PMCAs. The antibodies produced against human PMCAs can be used to detect human and animal PMCAs in an immunoassay if the degree of homology is high for PMCAs across human and animal species.

According to one embodiment, immunostaining comprises immunohistochemistry (IHC) which is the process of selectively identifying antigens (PMCAs) by antibodies binding specifically to PMCA in tissue samples. IHC is a well-known method to one skilled in the art. Visualizing an antibody-antigen interaction can be accomplished in a number of ways such as chromogenic immunohistochemistry (CIH), wherein an antibody is conjugated to an enzyme, such as peroxidase (the combination being termed immunoperoxidase), that catalyzes a color-producing reaction; or immunofluorescence, where the antibody is tagged to a fluorophore, such as fluorescein or rhodamine. In immunohistochemical detection methods, antibodies are classified as primary or secondary reagents. Primary antibodies are raised against an antigen of interest and are typically unconjugated (unlabeled), while secondary antibodies are raised against immunoglobulins of the primary antibody species. The secondary antibody is usually conjugated to a linker molecule, such as biotin, that then recruits reporter molecules, or the secondary antibody itself is directly bound to the reporter molecule. According to some embodiments, the secondary antibody against the anti-gamma actin primary antibody is conjugated to a red dye while the secondary antibody against the anti-PMCA primary antibody is conjugated to a green dye. Embodiments of the present disclosure provide methods for both direct and indirect immunohistochemical staining using antibodies against any of PMCA1-4. The direct method of immunohistochemical staining uses one labelled antibody, which binds directly to the PMCA antigen being probed for. The indirect method of immunohistochemical staining uses one first antibody against the antigen PMCA being probed for, and a second, labelled, antibody against the first antibody. Antibodies or antigen-binding fragments of the present disclosure may be coupled, i.e. covalently or non-covalently linked, to other moieties such as detectable labels. In some embodiments, the primary and/or secondary antibodies are coupled to at least one detectable label. The detectable label includes any enzymatic, chromogenic and/or fluorescent molecules known to one skilled in the art for labeling antibodies for detection.

According to some embodiments, the antibodies used for specific detection include polyclonal or monoclonal antibodies against PMCAs or their peptide fragments. Polyclonal antibodies are made by injecting animals with PMCA, or their peptide fragments and, after a secondary immune response is stimulated, isolating antibodies from whole serum. Polyclonal antibodies usually are a heterogeneous mix of antibodies that recognize several epitopes. Monoclonal antibodies are made by injecting the animal and then taking a specific sample of immune tissue, isolating a parent cell, and using the resulting immortalized line to create antibodies. Monoclonal antibodies show specificity for a single epitope.

Aspects of the present disclosure provide for methods of detecting cytocapsular tubes in a tissue of a subject. In one embodiment, the method includes obtaining a tissue from the subject, and detecting the presence of the cytocapsular tubes in the tissue by contacting the tissue with an anti-plasma membrane Ca²⁺-ATPase (PMCA) antibody. In one embodiment, the subject is a mammal. In one embodiment, the subject is a human or an animal. In another embodiment, the subject is healthy or diseased. In yet another embodiment, the subject is suffering from cancer or suspected to suffer from cancer. In certain embodiments, the tissue is obtained from anywhere in the body of the subject. In other embodiments, the tissue comprises healthy, benign, or cancerous tissue. In still other embodiments, the cancerous tissue comprises any tumor tissue comprising the primary site of tumor origin or the secondary site of tumor metastasis. In yet another embodiment, tissue adjacent to the cancerous tissue is obtained. Tissue adjacent to the cancerous tissues are also called “normal adjacent tissue” (NAT) by those skilled in the art. In one embodiment, the tissue is obtained by biopsy. In certain embodiments, the four isoforms of PMCAs includes PMCA-1, PMCA-2, PMCA-3 and PMCA-4 and their natural variants. In some embodiments, anti-PMCA antibodies include anti-PMCA-1 antibody, anti-PMCA-2 antibody, anti-PMCA-3 antibody, and anti-PMCA-4 antibody, and antibodies against their variants.

Certain aspects of the present disclosure provide for methods of predicting the grade of cancer metastasis of a subject. In one embodiment, the method includes obtaining a tissue from the subject, and detecting the presence of the cytocapsular tubes in the tissue. In one embodiment, detection of the cytocapsular tubes in the tissue is carried out by contacting the tissue with an anti-plasma membrane Ca²⁺-ATPase (PMCA) antibody. In certain embodiments, antibodies against the four isoforms of PMCAs including PMCA-1, PMCA-2, PMCA-3 and PMCA-4 and their natural variants are used for detection. In one embodiment, the presence of the cytocapsular tubes in the tissue is associated with metastasis cancer. In certain embodiments, the density and morphology of the cytocapsular tubes detected in the tissue is used to predict the grade of cancer metastasis of the subject. In one embodiment, increase in the density of cytocapsular tubes is associated with more advanced grade of cancer metastasis. In another embodiments, thinner and cloud-like cytocapsular tube morphology and more degradation is associated with more advanced grade of cancer metastasis. In one embodiment, if no cytocapsular tube (CCT) is detected and no CCT degradation is detected, cancer metastasis (CM) of grade 0 is predicted. In another embodiments, if the density of CCT detected is in the range of 1-10/mm² and no CCT degradation is detected, cancer metastasis (CM) of grade 1 is predicted. In one embodiment, if the density of CCT detected is in the range of 11-40/mm², and CCTs are degraded into strands and thin strands, cancer metastasis (CM) of grade 2 is predicted. In another embodiment, if the density of CCT detected is in the range of 41-80/mm², and CCTs are degraded into strands, thin strands, and silk-like strands, cancer metastasis (CM) of grade 3 is predicted. In one embodiment, if the density of CCT detected is in the range of >81/mm², and CCTs are degraded into cloud-like morphology or completely decomposed, cancer metastasis (CM) of grade 4 is predicted. In certain embodiments, the subject is a mammal, a human or an animal. In some embodiments, the subject is healthy or diseased. In other embodiments, the subject is suffering from cancer or suspected to suffer from cancer. In some embodiments, the tissue is obtained from anywhere in the body of the subject. In other embodiments, the tissue comprises healthy, benign, or cancerous tissue. In some embodiments, the cancerous tissue comprises any tumor tissue comprising the primary site of tumor origin or the secondary site of tumor metastasis.

Other aspects of the present disclosure provide for methods of screening a subject for cancer. In one embodiment, the method includes obtaining a tissue from the subject, and detecting the presence of the cytocapsular tubes in the tissue by contacting the tissue with an anti-plasma membrane Ca²⁺-ATPase (PMCA) antibody. In certain embodiments, antibodies against the four isoforms of PMCAs including PMCA-1, PMCA-2, PMCA-3 and PMCA-4 and their natural variants are used for detection. In one embodiment, the presence of the cytocapsular tubes in the tissue is an indication that the tissue is cancerous. In another embodiment, the absence of the cytocapsular tubes in the tissue is an indication that the tissue is healthy or benign. In certain embodiments, the subject is a mammal, a human or an animal. In some embodiments, the subject is healthy or diseased. In other embodiments, the subject is suffering from cancer or suspected to suffer from cancer. In some embodiments, the subject has been screened by other methods and determined to have or not to have cancer. The method provided herein can assist the cancer screening by other methods and provide more precise cancer diagnosis, detection or additional information that help cancer screening and diagnosis. In some embodiments, the tissue is obtained from anywhere in the body of the subject. In other embodiments, the tissue comprises healthy, benign, or cancerous tissue. In some embodiments, the cancerous tissue comprises any tumor tissue comprising the primary site of tumor origin or the secondary site of tumor metastasis.

Embodiments of the present disclosure are directed to methods of detecting cytocapsulae/cytocapsulas (CCs) and cytocapsular tubes (CCTs) in tissue samples from a subject or in 3D matrix culture in vitro. As used herein, the term “cytocapsulae” and “cytocapsulas” are used interchangeably to refer to the plural of cytocapsula. The terms “subject”, “individual”, or “patient” are used interchangeably and relate to vertebrates, preferably mammals. For example, mammals in the context of the present disclosure are humans, non-human primates, domesticated animals such as dogs, cats, sheep, cattle, goats, pigs, horses etc., laboratory animals such as mice, rats, rabbits, guinea pigs, etc. as well as animals in captivity such as animals of zoos. The term “animal” as used herein also includes humans. The term “subject” may also include a patient, i.e., an animal, preferably a human having a disease, preferably a disease such as cancer as described herein.

The term “normal” such as used in the terms “normal tissues” or “normal cells” refers to healthy tissues or cells in a healthy subject, i.e., non-pathological conditions, whereas “healthy” as used in the term “healthy subject” means non-cancerous.

The term the “tumor” as used in the terms “tumor cells” or “tumor tissues” refers to a swelling or lesion formed by an abnormal/uncontrolled growth of cells (called neoplastic cells or tumor cells). Tumors may be either benign or malignant. A benign tumor refers to a tumor that lacks all three of the malignant properties of a cancer, that is, a benign tumor does not grow in an unlimited, aggressive manner, does not invade surrounding tissues, and does not spread to non-adjacent tissues (metastasize). A malignant tumor refers to a tumor that displaces all three of the malignant properties of a cancer. A malignant tumor grows in an unlimited, aggressive manner, invades surrounding tissues, and spreads to non-adjacent tissues (metastasize). Cancerous cells display uncontrolled growth (division beyond the normal limits), invasion (intrusion on and destruction of adjacent tissues), and sometimes metastasis (spread to other locations in the body via lymph or blood). Most cancers form a tumor but some, like leukemia, do not.

The terms “malignant tumor progression”, “cancer progression”, and “cancer metastasis” are herein used interchangeably and refer to the stages of uncontrolled tumor cell growth, invading into the surrounding tissues, and spreading to secondary, non-adjacent tissues. “Tumor growth” as used herein relates to the tendency of a tumor to increase its size and/or to the tendency of tumor cells to proliferate.

The inventor of the present disclosure has found that upon malignant transformation, aggressive oncocells generate a second membrane outside the plasma membrane to form cytocapsulas (CCs) and cytocapsular tubes (CCTs), constituting cytocapsular oncocells, with pleotropic biological functions in cancer patient tissues in vivo and in vitro. These oncocells, aggressive oncocells, cancerous cells and malignant tumor cells are believed to have undergone malignant transformation and are capable of generating the second membrane-lined organelles, such as cytocapsulas (CCs) and cytocapsular tubes (CCTs) in vivo. The cytocapsulas (CCs) and cytocapsular tubes (CCTs) are lined by the second membrane, i.e., the cytocapsular membrane, that is different from the cell plasma membrane. Proteomics analysis revealed that the PMCA2 calcium pump is highly upregulated in the membranes of CCs and CCTs in malignant tumors but not in normal tissues, thus defining a unique cancer biomarker and target for cancer therapy. Cytocapsulas (CCs) and cytocapsular tubes (CCTs) are these second membrane-lined organelles.

The inventor of the present disclosure has found that cytocapsular oncocells are universally present in solid cancers and appear in hematologic cancers in immune organs. The term “cytocapsular oncocell” as used herein refer to a previously unrecognized single cancerous cell that is enclosed/enveloped in an extracellular second membrane of the cytocapsula, or cytocapsular tube in vivo and in vitro.

The inventor of the present disclosure has also found that multi-cell malignant tumors are also enveloped by the protective CC membranes, i.e., the cytocapsular membranes in vivo and in vitro. These cytocapsular membrane enclosed/enveloped multi-cell malignant tumors are interchangeably referred to herein as “cytocapsular tumors” or “cytocapsular tumorspheres”. It was observed that these cytocapsular tumors (CTs) generate numerous CCTs that form freeways for cancer cell metastasis to neighboring and far-distance destinations. Entire CT Networks (CTNs) dominate cancer metastasis physical pathways in cancer patients in vivo. Later, CCTs invade micro blood vessels and release cytocapsular oncocells into the blood providing a source of circulating malignant tumor cells. CTNs interconnect cytocapsular tumors in primary and secondary cancer niches creating larger Cytocapsular Tumor Network Systems (CTNSs). Primary and secondary CTNSs are connected and form dynamic and integrated CTNSs. Thus, interconnected cytocapsular oncocells, CTNs and CTNSs coordinate cancer progression in the integrated cytocapsular membrane systems.

Embodiments of the present disclosure provides a method of detecting cytocapsular tubes in a tissue sample from a subject. In one embodiment, the method includes contacting the tissue sample with an antibody, wherein the antibody binds to plasma membrane Ca²⁺-ATPase (PMCA) in cytocapsular membrane of the cytocapsular tubes, and detecting the presence of the cytocapsular tubes in the tissue sample by detecting the antibody. In another embodiment, the method includes detecting cytocapsulars, cytocapsulasome vesicles (cytocapsulasomes), cytocapsular oncocells, cytocapsular tumorspheres (cytocapsular tumors), and cytocapsular tumorsphere network systems in the tissue sample. The presence of the cytocapsular tubes and/or cytocapsulars in the tissue sample indicates the presence of or risk for (i.e. a potential for a development of) a cancer disease in the subject.

Embodiments of the present disclosure provides a method of detecting cytocapsular tubes in vitro. In one embodiment, the method includes contacting the cytocapsular tubes in a 3D matrix culture with an antibody, wherein the antibody binds to plasma membrane Ca²⁺-ATPase (PMCA) in cytocapsular membrane of the cytocapsular tubes, and detecting the presence of the cytocapsular tubes in the 3D matrix culture by detecting the antibody. In another embodiment, the method includes detecting cytocapsulars, cytocapsulasome vesicles (cytocapsulasomes), cytocapsular oncocells, cytocapsular tumorspheres (cytocapsular tumors), and cytocapsular tumorsphere network systems in the 3D matrix culture. The methods for the generation of cytocapsulas and cytocapsular tubes in 3D matrix culture in vitro were previously described (Tingfang Yi and Gerhard Wagner, Cytocapsular Tubes Conduct Cell Translocation. Proc Natl Acad Sci USA. 2018 Feb. 6; 115(6):E1137-E1146). The cytocapsulars, cytocapsular tubes, cytocapsulasome vesicles (cytocapsulasomes), cytocapsular oncocells, cytocapsular tumorspheres (cytocapsular tumors), and cytocapsular tumorsphere network systems all have cytocapsular membranes and are collectively referred to as cytocapsular membrane associated structures.

The present disclosure provides a kit for use in the method described herein. The kit comprises an antibody or antigen-binding fragment described herein, wherein the antibody or antigen-binding fragment binds to PMCA1-4 as described herein. The kit is useful in the methods of the present disclosure such as the methods for detection of cytocapsular tubes in vivo or in vitro. The kit may optionally comprise a detectable label, e.g. indicator enzymes, radiolabels, fluorophores, biotin, colored latex beads, gold nanoparticles, or metal tags. The kit may include informative pamphlets, for example, pamphlets informing on how to use reagents to practice a method disclosed herein.

Malignant tumor progression, cancer metastasis, cancer relapse, pan-cancer drug resistance and immune therapy escape present unmet challenges in cancer therapy. The mechanisms underlying cancer metastasis and progression in vivo are still unclear. The inventor of the present disclosure has found that cancerous cells generate cytocapsulas and cytocapsular tubes outside of the cytoplasm membrane and form protective membranous tunnel systems for shielded cancer cell proliferation, metastasis, and tumor growth. The membranes that are generated by the cancerous cells that envelop or encircle the cytocapsulas and cytocapsular tubes are herein referred to as cytocapsular membranes, which are not the cytoplasm membranes of the cancerous cells. It has been observed that cytocapsular oncocells are shielded from and coexist with immune cells in immune organs. A single incytocapsular oncocell or single malignant tumor cell can proliferate and form small oncocell masses composed of several oncocells in the CC lumens. At this stage, there is no CCT emerging from these small oncocell masses. As used herein, the term “prophase cytocapsular tumor” (PCT) refers to the small oncocell masses enclosed in the CC lumens with no CCTs emerging from these small oncocell masses. At a later stage, the CC enveloping these oncocell masses enlarges and generates CCTs extending into neighbor and far-distance tissues for CCT membrane sheltered and protected oncocell metastasis. As used herein, the terms “cytocapsular tumor” and “cytocapsular tumorsphere” (CT) refer to the oncocell masses enclosed in the CC lumens with CCTs emerging from these oncocell masses.

The atlas of cancer progression in vivo suggests that the 3D cytocapsular tube networks interconnect cytocapsular tumors in primary and secondary niches and engender dynamic and complex cytocapsular tumor network systems. The findings described in the present disclosure reveal that cytocapsular membrane systems conduct membrane-enclosed and protected cancer progression in vivo.

In a previous report, it was found that aggressive cancer cells engender a second membrane outside the plasma membrane of cytocapsula (CC) or cytocapsular tube (CCT) in vitro in 3D matrix culture (Yi T, Wagner G, Cytocapsular tubes conduct cell translocation. Proc Natl Acad Sci USA. 115, E1137-E1146 (2018)). The present disclosure provides an in-depth investigation of the biology of these previously unrecognized organelles and their links to cancer progression in vivo. We identified a biomarker Ca²⁺-ATPase 2 (PMCA2, or ATP2B2) for the CC/CCT membrane, which allowed us to reveal the lifecycle of cytocapsular oncocells both in vitro and in vivo (Jeong J, et al., PMCA2 regulates HER2 protein kinase localization and signaling and promotes HER2-mediated breast cancer. Proc Natl Acad Sci USA. 113, E282-90 (2016); Street A, et al., Mutations in a plasma membrane Ca²⁺-ATPase gene cause deafness in deafwaddler mice. Nat Genet. 19, 390-4 (1998); VanHouten J, et al., PMCA2 regulates apoptosis during mammary gland involution and predicts outcome in breast cancer. Proc Natl Acad Sci USA. 107, 11405-11410 (2010)). The present disclosure describes a series of previously undocumented cancer phenomena, such as: prophase cytocapsular tumors (PCTs), cytocapsular tumor (CT), Nuperphase Tumor (NT), acytocapsular oncocell mass-CC/CCT complex (AMCC), cytocapsulasome, cytocapsular tumor network system (CTNS), and integrated primary and secondary CTNSs. The results and observations of the present disclosure expand our understanding of cancer progression in vivo into systematic and cytocapsular membrane network-shielded oncocell devolution and evolution systems.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example I

### Detection of Endogenous PMCA-2 Expression in Cytocapsular Tubes in Normal, Benign and Primary Cancer Tissues.

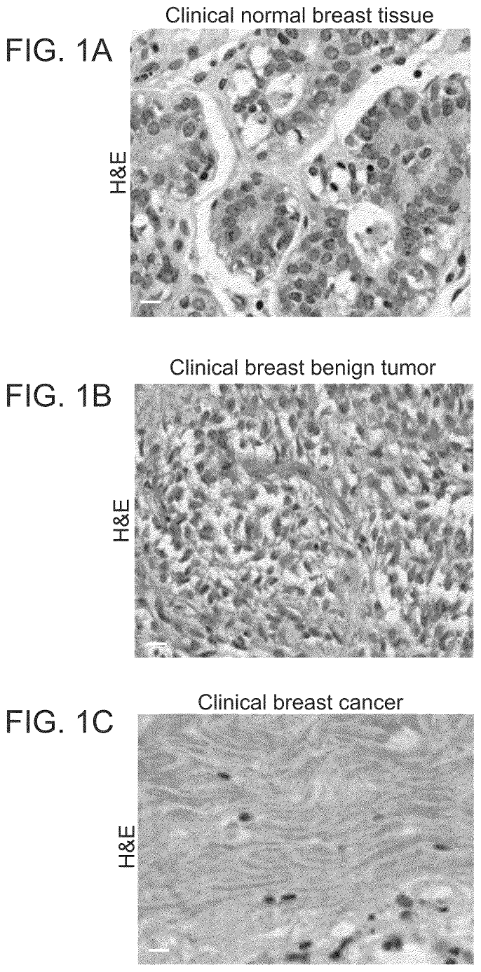

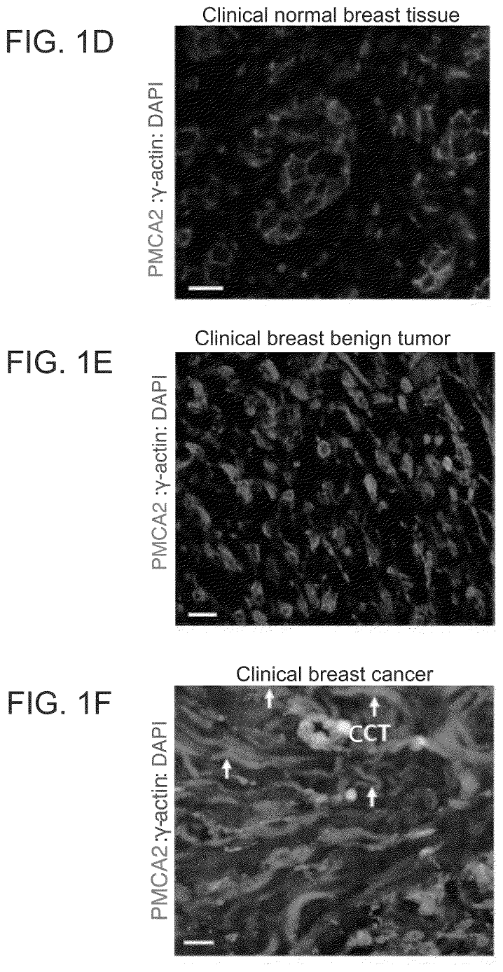

Embodiments of the present disclosure are directed to methods of detecting endogenous PMCA-2 expression in cytocapsular tubes in tissues. In some embodiments, whether normal cells, benign tumor cells and cancer cells in tissues obtained from human subjects can generate cytocapsular tubes in vivo were investigated. In some embodiments, H&E staining and immunohistochemistry (IHC) fluorescence staining analyses were performed with clinical normal breast tissues, benign breast tissues and breast cancer tissues (5 pieces of specimens from 5 different patients of each kind of tissues, 1 piece of specimen per patient; for the 5 breast cancer patients, 1 in stage I, 2 in stage II, 1 in stage III, and 1 in stage IV, surgical biopsy) together with rabbit polyclonal antibodies of anti-PMCA2 (Green) and mouse monoclonal antibodies of anti-gamma-Actin (Red) (FIGS. 1A-1F). No cytocapsular tube was detected in normal breast tissues or breast benign tumor tissues (Table 1). However, a large quantity of cytocapsular tubes (CCT, 3-6 μm in diameter, 300-4000 μm in length in sectioned specimens, 60-115 CTs/mm² in CCT density) were detected in all the tested breast cancer tissues, stages I-IV. These observations evidenced that breast cancer cells, but not normal breast cells or benign breast tumor cells, generate CCTs in breast cancer tissues in vivo. Interestingly, H&E staining assays did not show CCTs in the same tissue samples (FIGS. 1A-1F). These data suggested that while the presence of CCTs are invisible/non-detectable by H&E staining assays, immunohistochemistry (IHC) fluorescence staining with antibodies against PMCA2 can detect the expression of PMCA2 proteins in CCT membranes, and is a sensitive marker specific for CCTs in cancer tissues.

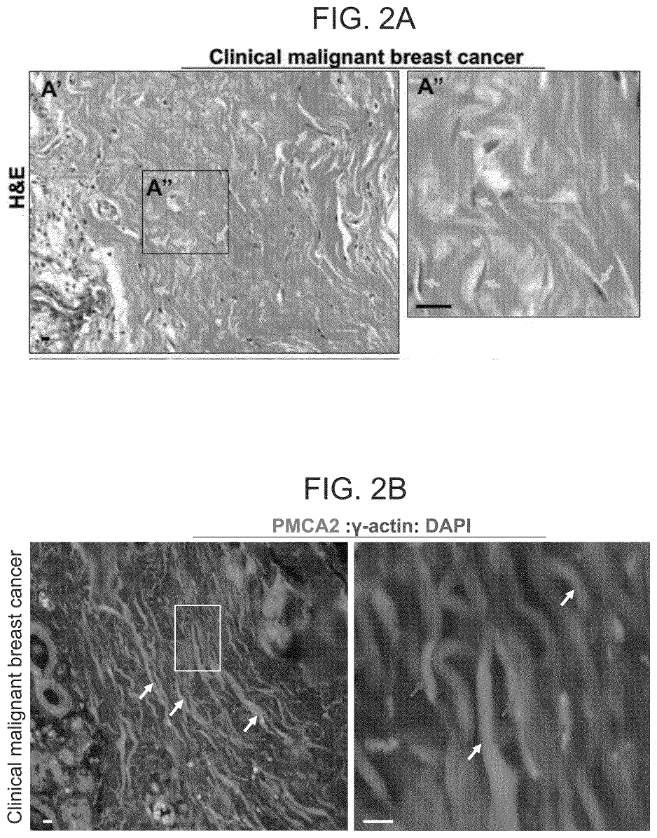

Next, whether cancer cells migrate in CCTs in cancer tissues were examined. H&E staining assays and immunohistochemistry (IHC) fluorescence staining analyses were performed with clinical breast cancer tissues (5 pieces of specimens from 5 different breast cancer patients in stage II, 1 piece of specimen per patient, surgical biopsy) together with rabbit polyclonal antibodies of anti-PMCA2 (Green) and mouse monoclonal antibodies of gamma-actin (Red) (FIGS. 2A-2B). As expected, there are a lot of CTs (60-110 CCTs/mm² in CCT density) in clinical stage II breast cancer tissues, and many cancer cells in CCTs display long and narrow spindle-shaped morphologies exposed in sectioned CCTs, suggesting that these cancer cells are migrating in CCTs and use CCTs as membrane enclosed tube-shaped freeways as means for metastasis in compact and crowded cancer tissues (FIGS. 2A-2B).

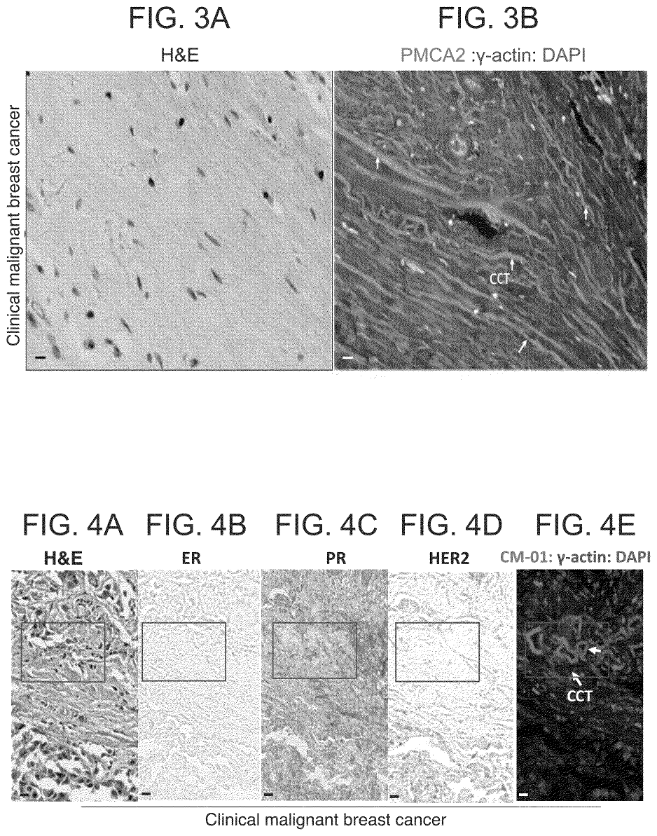

Next, CCT density and morphology in clinical breast cancer tissues in later clinical stages were investigated (5 pieces of specimens from 5 different breast cancer patients in stage III, 1 piece of specimen per patient, surgical biopsy) together with rabbit polyclonal antibodies of anti-PMCA2 (Green) and mouse monoclonal antibodies of gamma-actin (Red) (FIGS. 3A-3B). There are plenty of (75-126 CCTs/mm² in CCT density) long and curved and entangled CCTs in stage III breast cancer tissues (FIGS. 3A-3B). The above results (FIGS. 1-3, and Table 2) suggested that cancer cells in later stage clinical cancer tissues generate increased number of long and membrane enclosed CCTs. The increase in CCT numbers/densities seems to correlate with the degree of cancer metastasis for these cancer tissues.

In order to compare the sensitivity of breast cancer tumor markers, conventional H&E staining assays and IHC assays with antibodies recognizing breast cancer molecular markers of ER, PR, HER-2, and IHC-fluorescence assays with antibodies of anti-PMCA2 recognizing protein PMCA2 together with 5 continuous specimen sections of breast cancer tissues of clinical stage III were performed (FIGS. 4A-4E). Only IHC-fluorescence assays with antibodies of anti-PMCA2 showed the detection of CCTs in clinical breast cancer tissue. Conventional H&E staining assays did not show any CCTs in breast cancer tissues. IHC staining with antibodies recognizing breast cancer molecular markers of ER, PR, and HER-2 showed these breast cancers as either positive or negative with their respective markers ER, PR and HER-2 with various signal intensity and they failed to show CCTs (FIGS. 4B-4D). These data (FIGS. 1-4) strongly suggested that IHC-fluorescence assay with antibodies of anti-PMCA2 is a much more sensitive assay for detection of breast cancer compared to conventional H&E staining assays and IHC assays with antibodies recognizing breast cancer molecular markers of ER, PR, and HER-2, and can provide more sensitivity and precision by detection of CCTs in breast cancer tissues useful for breast cancer diagnosis, prognosis and monitoring.

TABLE 1
A list of clinical normal human tissue samples
showing no detection of CCTs in these tissues.

| Tissue | Number of Positive CCT Staining | Total Number Tested |
|---|---|---|
| Breast | 0 | 3 |
| Liver | 0 | 2 |
| Colon | 0 | 2 |
| Stomach | 0 | 3 |
| Lung | 0 | 3 |
| Esophagus | 0 | 2 |
| Prostate | 0 | 1 |

Table 2. A summary of breast cancer cell cytocapsular tube analyses in clinical patient breast cancer tissues by immunohistochemistry (IHC) with anti-PMCA2, anti-ER, PR and HER2 antibodies, and anti-gamma-actin antibodies. The cytocapsular tube (CCT) number, degradation status and cancer metastasis grades are shown.

| | | | | | | | | | | | | Cytocap- | | | |
|---|---|---|---|---|---|---|---|---|---|---|---|---|---|---|---|
| | | | Organ/ | | | | | | | | | sular | | | Cancer |
| | | | Anatomic | Pathology | | | | | | | HER2 | Tubes | CT/ | CT | metastasis |
| No. | Age | Sex | Site | diagnosis | TNM | Grade | Stage | Type | ER | PR | Ki67 | (CT) | mm2 | degradation | grade |
| 1 | 48 | F | Breast | Invasive | T1NOMO | 1 | I | Malig- | + | − | − | + | 6 | − | 1 |
| | | | | ductal | | | | nant | | | | | | | |
| | | | | carcinoma | | | | | | | | | | | |
| 2 | 64 | F | Breast | Invasive | TINOMO | 1 | I | Malig- | +++ | − | − | + | 9 | − | 1 |
| | | | | ductal | | | | nant | | | | | | | |
| | | | | carcinoma | | | | | | | | | | | |
| 3 | 64 | F | Breast | Invasive | T1NOMO | 1 | I | Malig- | +++ | − | − | + | 6 | − | 1 |
| | | | | ductal | | | | nant | | | | | | | |
| | | | | carcinoma | | | | | | | | | | | |
| 4 | 50 | F | Breast | Invasive | T1NOMO | 1 | I | Malig- | − | * | 1+ | + | 8 | − | 1 |
| | | | | ductal | | | | nant | | | | | | | |
| | | | | carcinoma | | | | | | | | | | | |
| 5 | 61 | F | Breast | Invasive | TINOMO | 1 | I | Malig- | ++ | + | − | + | 24 | − | 2 |
| | | | | ductal | | | | nant | | | | | | | |
| | | | | carcinoma | | | | | | | | | | | |
| 6 | 61 | F | Breast | Invasive | T1NOMO | 1 | I | Malig- | ++ | + | − | + | 18 | − | 2 |
| | | | | ductal | | | | nant | | | | | | | |
| | | | | carcinoma | | | | | | | | | | | |
| 7 | 48 | F | Breast | Invasive | T1NOMO | 1 | I | Malig- | + | − | − | + | 12 | − | 2 |
| | | | | ductal | | | | nant | | | | | | | |
| | | | | carcinoma | | | | | | | | | | | |
| 8 | 50 | F | Breast | Invasive | T1NOMO | 1 | I | Malig- | − | * | 1+ | + | 13 | − | 2 |
| | | | | ductal | | | | nant | | | | | | | |
| | | | | carcinoma | | | | | | | | | | | |
| 9 | 40 | F | Breast | Invasive | TINOMO | 1 | I | Malig- | + | * | 0 | + | 16 | − | 2 |
| | | | | ductal | | | | nant | | | | | | | |
| | | | | carcinoma | | | | | | | | | | | |
| 10 | 40 | F | Breast | Invasive | TINOMO | 1 | I | Malig- | + | * | 0 | + | 22 | − | 2 |
| | | | | ducta | | | | nant | | | | | | | |
| | | | | carcinoma | | | | | | | | | | | |
| 11 | 56 | F | Breast | Invasive | TINOMO | 1 | I | Malig- | +++ | * | 0 | + | 27 | − | 2 |
| | | | | ducta | | | | nant | | | | | | | |
| | | | | carcinoma | | | | | | | | | | | |
| 12 | 56 | F | Breast | Invasive | TINOMO | 1 | I | Malig- | +++ | * | 0 | + | 11 | − | 2 |
| | | | | ductal | | | | nant | | | | | | | |
| | | | | carcinoma | | | | | | | | | | | |
| 13 | 33 | F | Breast | Invasive | T1NOMO | 1 | I | Malig- | +++ | * | 0 | + | 38 | − | 2 |
| | | | | ducta | | | | nant | | | | | | | |
| | | | | carcinoma | | | | | | | | | | | |
| 14 | 67 | F | Breast | Invasive | TINOMO | 1 | I | Malig- | ++ | ++ | 0 | + | 38 | − | 2 |
| | | | | ducta | | | | nant | | | | | | | |
| | | | | carcinoma | | | | | | | | | | | |
| 15 | 33 | F | Breast | Invasive | T1NOMO | 1 | I | Malig- | +++ | * | 0 | + | 46 | + | 3 |
| | | | | ductal | | | | nant | | | | | | | |
| | | | | carcinoma | | | | | | | | | | | |
| 16 | 48 | F | Breast | Invasive | TINOMO | 1 | I | Malig- | + | − | 0 | + | 51 | − | 3 |
| | | | | ductal | | | | nant | | | | | | | |
| | | | | carcinoma | | | | | | | | | | | |
| 17 | 48 | F | Breast | Invasive | T1NOMO | 1 | I | Malig- | + | * | 0 | + | 42 | + | 3 |
| | | | | ductal | | | | nant | | | | | | | |
| | | | | carcinoma | | | | | | | | | | | |
| 18 | 55 | F | Breast | Invasive | TINOMO | 2 | I | Malig- | − | − | − | + | 45 | − | 3 |
| | | | | ductal | | | | nant | | | | | | | |
| | | | | carcinoma | | | | | | | | | | | |
| 19 | 55 | F | Breast | Invasive | T1NOMO | 2 | I | Malig- | − | − | − | + | 34 | − | 2 |
| | | | | ductal | | | | nant | | | | | | | |
| | | | | carcinoma | | | | | | | | | | | |
| 20 | 69 | F | Breast | Invasive | T1NOMO | 2 | I | Malig- | +++ | +++ | − | + | 56 | + | 3 |
| | | | | ductal | | | | nant | | | | | | | |
| | | | | carcinoma | | | | | | | | | | | |

(Note:
F, female; ″+″, detected; ″−″, not detected; T1, tumor invades submucosa; T2, Tumor invades muscularis propria; T3, Tumor invades through muscularis propria into submucosa or into non-peritonealized pericolic or perirectal tissues; T4, Tumor directly invades other organs or structures and/or perforate visceral peritoneum. NO, no regional lymph node metastasis; N1, metastasis in 1 to 3 regional lymph nodes; N2, metastasis in 4 or more regional lymph nodes. MO, No distant metastasis; M1, Distant metastasis. CT/CCT, cytocapsular tube).

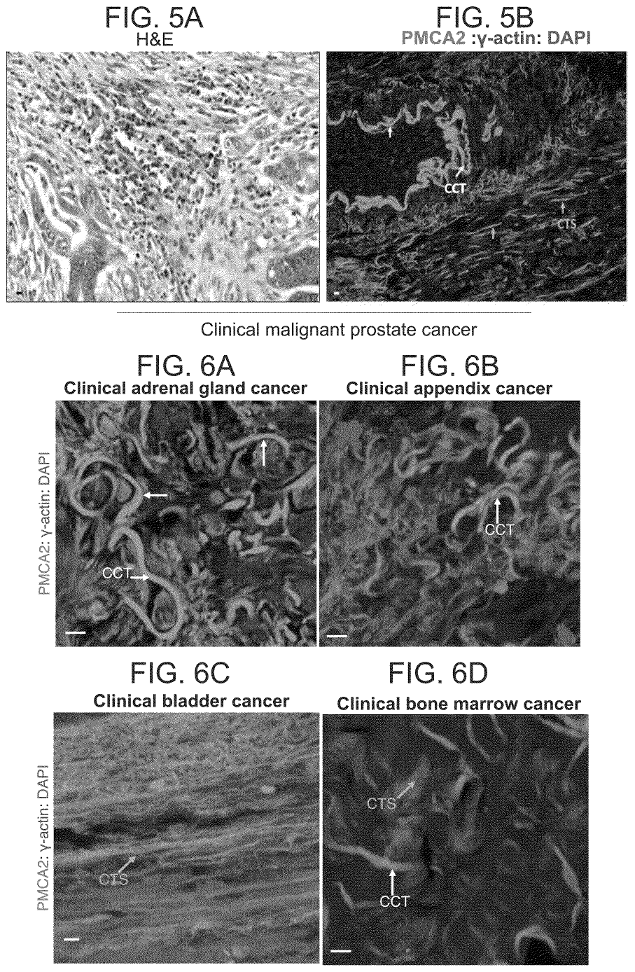

Prostate cancer is a top 5 cancer in men. Next, CCTs in clinical prostate cancer tissues were investigated (5 pieces of specimens from 5 different prostate cancer patients, 2 in stage I, 1 in stage II, 1 in stage III, and 1 in stage IV; 1 piece of specimen per patient, surgical biopsy) by H&E staining and IHC-fluorescence assays together with rabbit polyclonal antibodies of anti-PMCA2 (Green) and mouse monoclonal antibodies of gamma-actin (Red) (FIGS. 5A-5B). IHC-fluorescence assays with antibodies of anti-PMCA2, but not H&E staining assays, showed that there are plenty of (55-123 CCTs/mm² in CCT density) long and curved and entangled CCTs in clinical prostate cancer tissues of stage II (FIGS. 5A-5B). These data suggested that IHC-fluorescence assays with antibodies of anti-PMCA2 to detect CCTs in prostate cancer tissues can be used as a sensitive method in prostate cancer diagnosis, prognosis and monitoring.

### Example II

### Detection of Endogenous PMCA-2 Expression in Cytocapsular Tubes in Primary Cancer Tissues.

Cancers can arise from anywhere in a subject's body. The detection of CCTs in breast cancer and prostate cancer tissues strongly suggest that CCTs can be generated by cells in other types of cancers. Next, the CCTs in another 32 types of primary cancers from various organs and tissues were investigated using the method of IHC-fluorescence assays with antibodies of anti-PMCA2. Shown here are the results from investigated adrenal gland cancer, appendix cancer, bladder cancer and bone marrow cancer (5 pieces of specimens from 5 different cancer patients in each type of cancer, 2 in stage I, 1 in stage II, 1 in stage III, and 1 in stage IV; 1 piece of specimen per patient, surgical biopsy) with IHC-fluorescence assays with antibodies of anti-PMCA2. A lot of CCTs of variant densities (45-118 CCTs/mm² in CCT density) have been detected in all these cancer tissues investigated. As the metastasis progress to later clinical stages, some CCTs in the cancer tissues exhibited degraded morphology, progressing from thick, to thin and very thin strands through the CCT life cycle (FIGS. 6A-6D). Some degraded CCTs exhibited cloud-like morphology in very late stage (clinical stage IV). The cloud-like morphology corresponds to the end stage of CCT lifecycle.

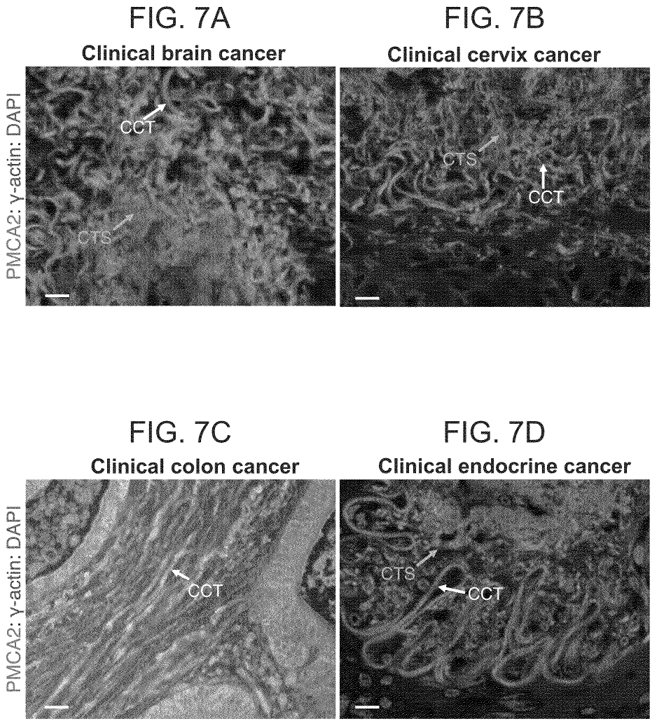

The investigation of CCTs were next expanded in additional cancer types including brain cancer, cervix cancer, colon cancer and endocrine cancer (5 pieces of specimens from 5 different cancer patients in each type of cancer, 1 in stage I, 1 in stage II, 2 in stage III, and 1 in stage IV; 1 piece of specimen per patient, surgical biopsy) with IHC-fluorescence assays with antibodies of anti-PMCA2. A lot of CCTs in variant densities (56-121 CCTs/mm² in CCT density) have been detected in all these cancer types. As the metastasis progress to later clinical stages, some CCTs in the cancer tissues exhibited degraded morphology, progressing from thick, to thin and very thin strands through the CCT life cycle (FIGS. 7A-7D).

Table 3. A summary of colon cancer tissue of difference cancer types or subtypes, showing cytocapsular tube analyses in these clinical patient colon cancer tissues by immunohistochemistry (IHC) with anti-PMCA2 antibodies. The cytocapsular tube (CCT/CT) number, degradation status and cancer metastasis grades are shown.

| | | | Organ/ | | | | | | Cytocapsul | | | Cancer |
|---|---|---|---|---|---|---|---|---|---|---|---|---|
| | | | Anatomic | Pathology | | | | | ar Tubes | CT/ | CT | metastasis |
| No. | Age | Sex | Site | diagnosis | TNM | Grade | Stage | Type | (CT) | mm2 | degradation | grade |
| 1 | 49 | M | Colon | Adenocarcinoma | T2NOMO | 1 | I | Malignant | + | 23 | + | 2 |
| 2 | 24 | F | Colon | Mucinous | T2NOMO | 1 | I | Malignant | + | 18 | + | 2 |
| | | | | Adenocarcinoma | | | | | | | | |
| 3 | 30 | M | Colon | Adenocarcinoma | TINOMO | 1 | I | Malignant | + | 27 | + | 2 |
| 4 | 27 | M | Colon | Adenocarcinoma | T2NOMO | 1 | I | Malignant | + | 31 | + | 2 |
| 5 | 69 | M | Colon | Adenocarcinoma | T2NOMO | 2 | I | Malignant | + | 27 | + | 2 |
| 6 | 51 | M | Colon | Mucinous | T2NOMO | 1 | I | Malignant | + | 39 | + | 2 |
| | | | | Adenocarcinoma | | | | | | | | |
| 7 | 68 | M | Colon | Adenocarcinoma | T2NOMO | 1 | I | Malignant | + | 43 | + | 3 |
| 8 | 45 | M | Colon | Mucinous | T2NOMO | 1 | I | Malignant | + | 52 | + | 3 |
| | | | | Adenocarcinoma | | | | | | | | |
| 9 | 63 | F | Colon | Adenocarcinoma | T2NOMO | 2 | I | Malignant | + | 33 | + | 2 |
| 10 | 69 | F | Colon | Adenocarcinoma | T2NOMO | 2 | I | Malignant | + | 29 | + | 2 |
| 11 | 49 | F | Colon | Adenocarcinoma | T2NOMO | 2 | I | Malignant | + | 46 | + | 3 |
| 12 | 67 | F | Colon | Adenocarcinoma | T2NOMO | 1 | I | Malignant | + | 31 | + | 2 |
| 13 | 53 | F | Colon | Adenocarcinoma | T2NOMO | 2 | I | Malignant | + | 44 | + | 3 |
| 14 | 69 | M | Colon | Adenocarcinoma | T2NOMO | 2 | I | Malignant | + | 38 | + | 2 |
| 15 | 64 | M | Colon | Mucinous | T2NOMO | 3 | I | Malignant | + | 52 | + | 3 |
| | | | | Adenocarcinoma | | | | | | | | |
| 16 | 61 | F | Colon | Adenocarcinoma | T2NOMO | 2 | I | Malignant | + | 63 | + | 3 |
| 17 | 70 | F | Colon | Mucinous | T3NOMO | 1 | I | Malignant | + | 46 | + | 3 |
| | | | | Adenocarcinoma | | | | | | | | |
| 18 | 57 | F | Colon | Adenocarcinoma | T2NOMO | 2 | I | Malignant | + | 38 | + | 2 |
| 19 | 43 | M | Colon | Mucinous | T2NOMO | 1 | I | Malignant | + | 29 | + | 2 |
| | | | | Adenocarcinoma | | | | | | | | |
| 20 | 51 | M | Colon | Mucinous | T2NOMO | 2 | I | Malignant | + | 35 | + | 2 |
| | | | | Adenocarcinoma | | | | | | | | |

(Note:
F, female; M, male; ″+″, detected; ″−″, not detected; T1, tumor invades submucosa; T2, Tumor invades muscularis propria; T3, Tumor invades through muscularis propria into submucosa or into non-peritonealized pericolic or perirectal tissues; T4, Tumor directly invades other organs or structures and/or perforate visceral peritoneum. NO, no regional lymph node metastasis; N1, metastasis in 1 to 3 regional lymph nodes; N2, metastasis in 4 or more regional lymph nodes. MO, No distant metastasis; M1, Distant metastasis. CT/CCT, cytocapsular tube).

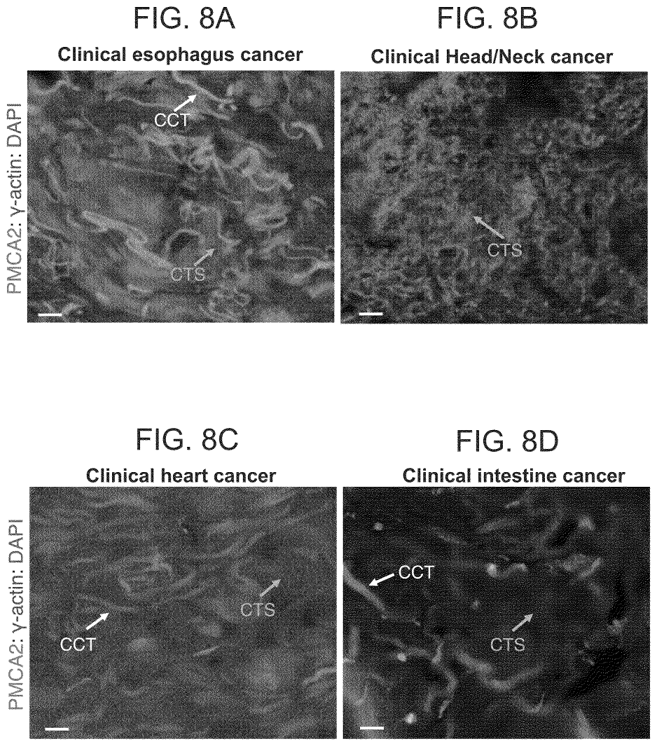

The investigation of CCTs were further expanded to esophagus cancer, head/neck cancer, heart cancer and intestine cancer (5 pieces of specimens from 5 different cancer patients in each type of cancer, 1 in stage I, 2 in stage II, 1 in stage III, and 1 in stage IV; 1 piece of specimen per patient, surgical biopsy) with IHC-fluorescence assays with antibodies of anti-PMCA2. A lot of CCTs in variant densities (54-118 CCTs/mm² in CCT density) have been detected in all these cancer types. As the metastasis progress to later clinical stages, some CCTs in the cancer tissues exhibited degraded morphology, progressing from thick, to thin and very thin strands through the CCT life cycle (FIGS. 8A-8D).

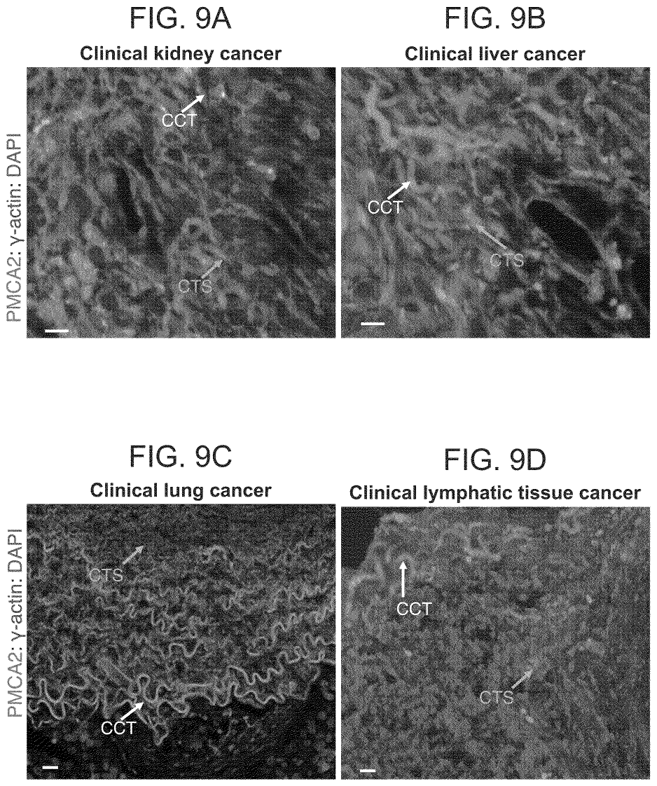

The investigation of CCTs were further expanded to kidney cancer, liver cancer, lung cancer and lymphatic tissue cancer (5 pieces of specimens from 5 different cancer patients in each type of cancer, 1 in stage I, 1 in stage II, 2 in stage III, and 1 in stage IV; 1 piece of specimen per patient, surgical biopsy) with IHC-fluorescence assays with antibodies of anti-PMCA2. A lot of CCTs in variant densities (48-127 CCTs/mm² in CCT density) have been detected in all these cancer types. As the metastasis progress to later clinical stages, some CCTs in the cancer tissues exhibited degraded morphology, progressing from thick, to thin and very thin strands through the CCT life cycle (FIGS. 9A-9D).

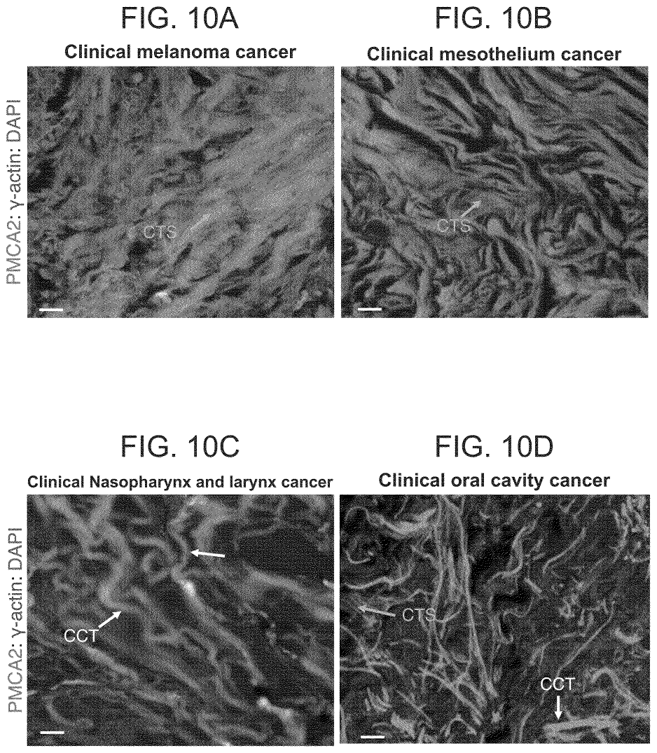

The investigation of CCTs were further expanded to melanoma cancer, mesothelium cancer, nasopharynx and larynx cancer and oral cavity cancer (5 pieces of specimens from 5 different cancer patients in each type of cancer, 1 in stage I, 1 in stage II, 1 in stage III, and 2 in stage IV; 1 piece of specimen per patient, surgical biopsy) with IHC-fluorescence assays with antibodies of anti-PMCA2. A lot of CCTs in variant densities (54-108 CCTs/mm² in CCT density) have been detected in all these cancer types. As the metastasis progress to later clinical stages, some CCTs in the cancer tissues exhibited degraded morphology, progressing from thick, to thin and very thin strands through the CCT life cycle (FIGS. 10A-10D).

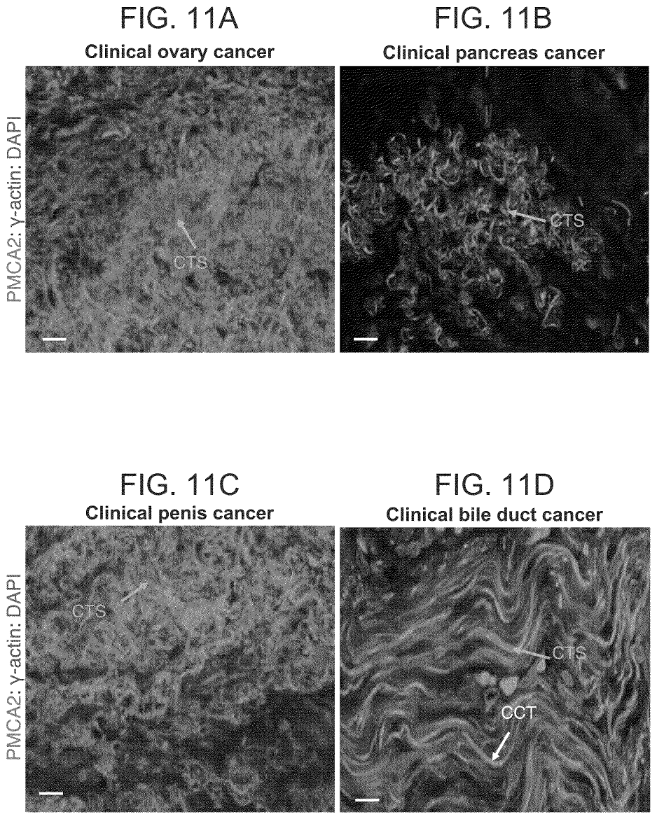

The investigation of CCTs were further expanded to ovary cancer, pancreas cancer, penis cancer and bile duct cancer (5 pieces of specimens from 5 different cancer patients in each type of cancer, 1 in stage I, 1 in stage II, 1 in stage III, and 2 in stage IV; 1 piece of specimen per patient, surgical biopsy) with IHC-fluorescence assays with antibodies of anti-PMCA2. A lot of curved and entangled CCTs in variant densities (47-107 CCTs/mm² in CCT density) have been detected in all these cancer types. As the metastasis progress to later clinical stages, some CCTs in the cancer tissues exhibited degraded morphology, progressing from thick, to thin and very thin strands through the CCT life cycle, with cloud-like morphology CCTs correspond to the end stage of CCT life cycle (FIGS. 11A-11D).

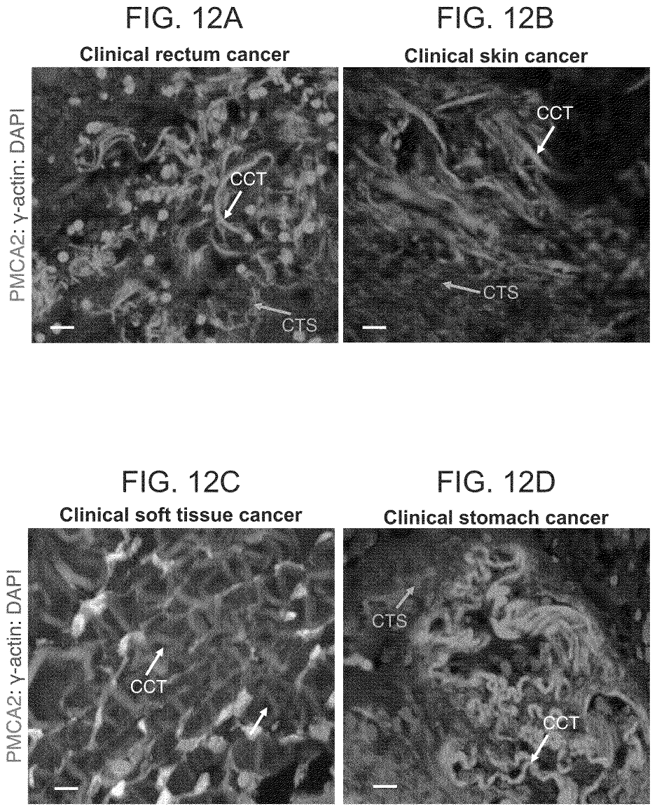

The investigation of CCTs were further expanded to rectum cancer, skin cancer, soft tissue (smooth muscle) cancer and stomach cancer (5 pieces of specimens from 5 different cancer patients in each type of cancer, 1 in stage I, 2 in stage II, 1 in stage III, and 1 in stage IV; 1 piece of specimen per patient, surgical biopsy) with IHC-fluorescence assays with antibodies of anti-PMCA2. A lot of CCTs in variant densities (44-118 CCTs/mm² in CCT density have been detected in all these cancer types. As the metastasis progress to later clinical stages, some CCTs in the cancer tissues exhibited degraded morphology, progressing from thick, to thin and very thin strands through the CCT life cycle, with cloud-like morphology CCTs correspond to the end stage of CCT life cycle (FIGS. 12A-12D).

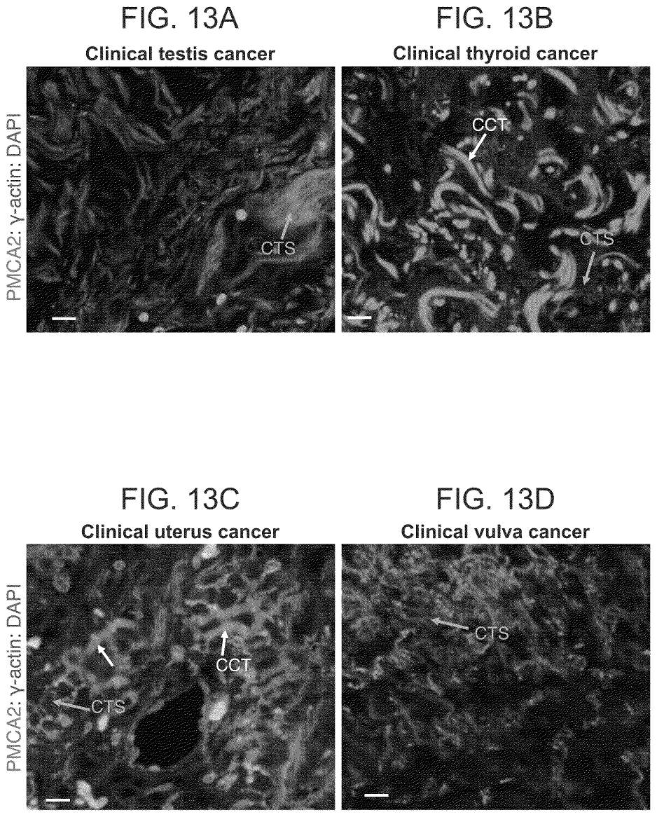

The investigation of CCTs were further expanded to testis cancer, thyroid cancer, uterus cancer and vulva cancer (5 pieces of specimens from 5 different cancer patients in each type of cancer, 1 in stage I, 1 in stage II, 2 in stage III, and 1 in stage IV; 1 piece of specimen per patient, surgical biopsy) with IHC-fluorescence assays with antibodies of anti-PMCA2. A lot of CCTs in variant densities (34-108 CCTs/mm² in CCT density) As the metastasis progress to later clinical stages, some CCTs in the cancer tissues exhibited degraded morphology, progressing from thick, to thin and very thin strands through the CT life cycle, with cloud-like morphology CTs correspond to the end stage of CCT life cycle (FIGS. 13A-13D).

### Example III

### Detection of CTs in Paracancer Tissue

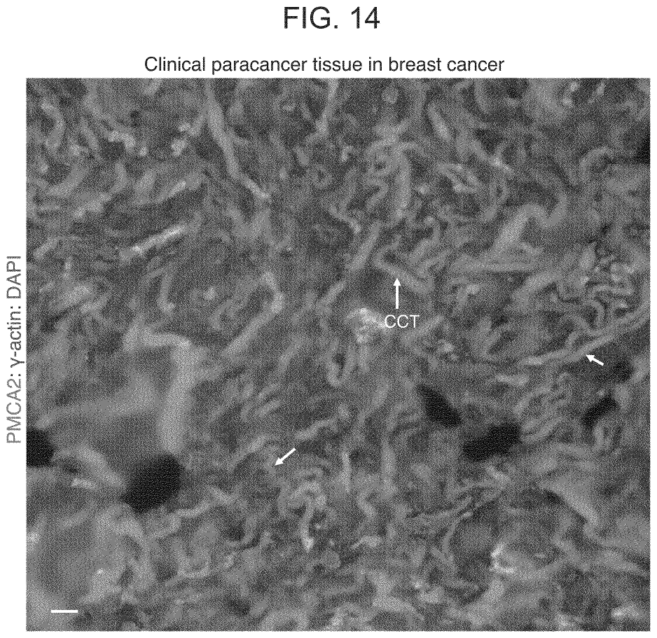

Paracancer tissue are conventionally called normal adjacent tissues (NAT). Then, whether there are CCTs in paracancer tissues were investigated. Paracancer tissue of breast cancer were examined (5 pieces of specimens from 5 different cancer patients of breast cancer, 1 in stage I, 1 in stage II, 2 in stage III, and 1 in stage IV; 1 piece of specimen per patient, surgical biopsy) with IHC-fluorescence assays with antibodies of anti-PMCA2. Surprisingly, a large quantity of long, curved and entangled CCTs (95-128 CCTs/mm² in CCT density) (FIG. 14 shows NAT of stage II breast cancer) were detected in these tissues of all stages of breast cancer examined. These data evidenced that the conventional NAT can actually be not normal tissues in a sense that it still harbors a lot of CCTs, presumably extended from the adjacent cancer tissues. The observations that paracancer tissues contain plenty of CCTs are consistent with the fact that CCTs extend through the paracancer tissues and function to conduct cancer cells from the primary site into neighbor and far distance organs and tissues. This observation is useful for re-assessing the area adjacent of cancer tissues for removal during cancer monitoring and treatment and for the extend of cancer spread during metastasis.

### Example IV

### Detection of CTs in Secondary Cancers

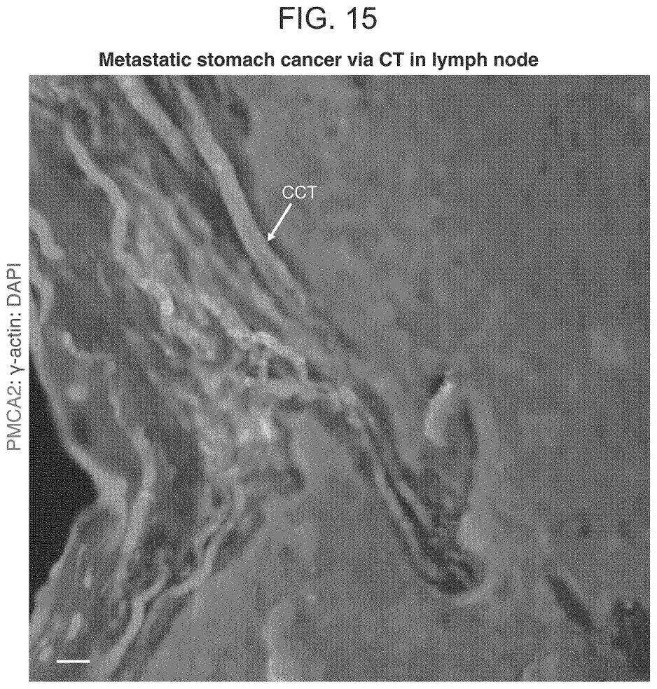

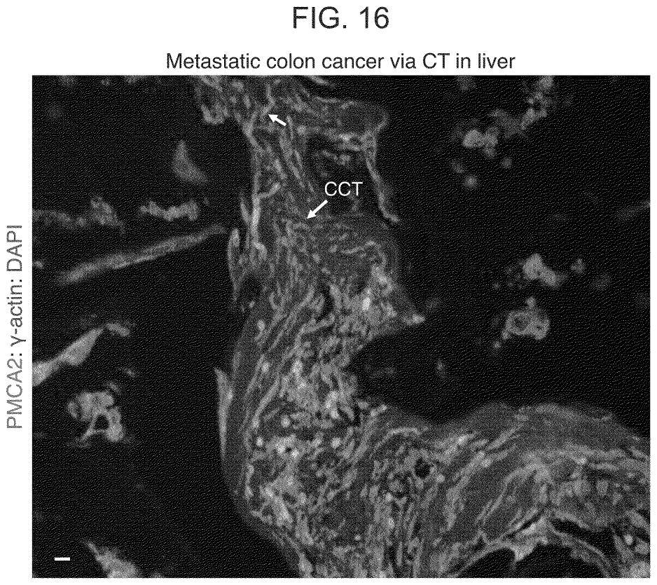

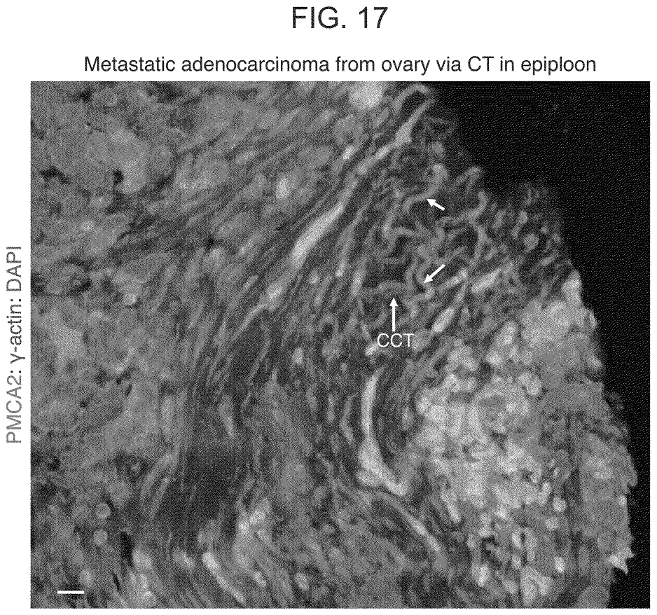

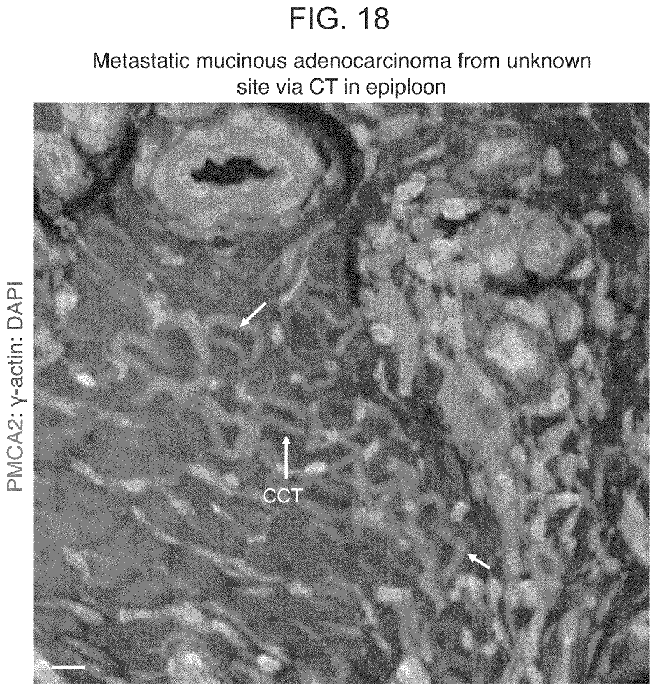

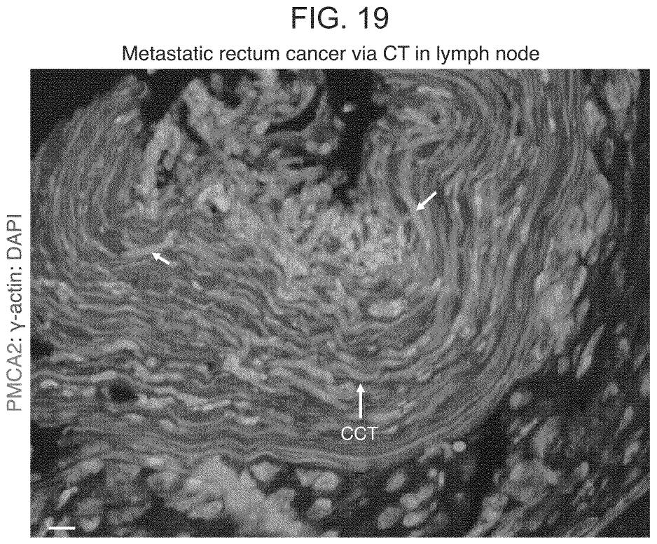

Cancers can metastasize from the primary site of origin to secondary sites of metastasis. Cancer metastasis leads to cancer cell dissemination to neighbor and far distance tissues and organs causing multiple organ biological functional failure and ultimately death. CCTs were thus investigated in multiple metastatic cancers in far distance tissues and organs (5 pieces of specimens from 5 different cancer patients of metastatic cancer, 3 in stage III, and 2 in stage IV; 1 piece of specimen per patient, surgical biopsy) with IHC-fluorescence assays with antibodies of anti-PMCA2. They are metastatic stomach cancer via CCT into lymph nodes (FIG. 15), metastatic colon cancer via CCT into liver (FIG. 16), metastatic adenocarcinoma from ovary via CCT into epiploon (FIG. 17), metastatic mucinous adenocarcinoma from unknown site via CCT into epiploon (FIG. 18) and metastatic rectum cancer via CCT into lymph nodes (FIG. 19). As expected, a lot of CTs (92-124 CCTs/mm² in CCT density) were detected in the metastatic cancers in far distance organs and tissues in all the tested samples. The above consistent data (FIGS. 1-19, and Tables 1-4) evidenced that cancer cells generate CCTs for cancer cell migration, dissemination and cancer metastasis in all kinds of clinical solid cancers and that the method of IHC-fluorescence assays with antibodies of anti-PMCA2 is reliable and applicable for detection of CCTs in cancer tissues and for prediction of cancer metastasis with great sensitivity and precision useful in all kinds of solid tumor cancers.

Embodiments of the present disclosure are directed to using the present methods to investigate CCTs in a wide variety of cancer types and subtypes. Table 4 provides an incomplete list of cancer tissues examined. Almost all tested cancer tissue samples are positive for PMCA-2 staining and positive for CCTs. The data suggested that the present methods for detecting PMCA-2 and CCTs in cancer tissues can be applied to all types of cancers.

TABLE 4
A list of cancer tissues with cytocapsular tubes (CCTs)
examined by IHC with anti-PMCA2 antibodies.

| | | Total number |
|---|---|---|
| | Number of | of tissue |
| | CCT positive | samples |
| Tissues Examined | samples | examined |
| Breast invasive ductal carcinoma | 2617 | 2617 |
| Breast medullary carcinoma | 54 | 57 |
| Breast mucinous carcinoma | 32 | 35 |
| Breast Fibroadenoma | 20 | 22 |
| Benign breast tumor | 2 | 25 |
| Metastatic breast cancer in lymph node | 33 | 33 |
| Metastatic breast cancer in lung | 4 | 4 |
| Metastatic breast cancer in liver | 7 | 7 |
| Paracancer of malignant breast cancer | 56 | 56 |
| Colon adenocarcinoma (colon cancer) | 76 | 80 |
| Colon mucinous adenocarcinoma | 23 | 25 |
| Colon squamous cell carcinoma | 9 | 10 |
| Metastatic colon cancer in lymph node | 5 | 5 |
| Metastatic colon cancer in liver | 4 | 4 |
| Metastatic colon cancer in epiploon | 3 | 3 |
| Metastatic colon cancer in ovary | 3 | 3 |
| Paracancer of malignant colon cancer | 21 | 21 |
| Pancreas adenocarcinoma | 20 | 21 |
| Pancreas ductal adenocarcinoma | 6 | 6 |
| Pancreas mucinous adenocarcinoma | 2 | 2 |
| Metastatic pancreas cancer in lymph | 3 | 3 |
| node | | |
| Metastatic pancreas cancer in mesentery | 2 | 2 |
| Paracancer of malignant pancreas cancer | 15 | 15 |
| Esophagus squamous cell carcinoma | 40 | 42 |
| Esophagus adenocarcinoma | 10 | 10 |
| Esophagus small cell carcinoma | 11 | 11 |
| Esophagus mucinous adenocarcinoma | 2 | 2 |
| Metastatic esophagus adenocarcinoma | 14 | 14 |
| to lymph node | | |
| Paracancer of esophagus | 10 | 10 |
| adenocarcinoma | | |
| Stomach adenocarcinoma | 32 | 34 |
| Stomach mucinous adenocarcinoma | 19 | 20 |
| Metastatic stomach cancer in lymph | 6 | 6 |
| node | | |
| Metastatic stomach cancer in liver | 2 | 2 |
| Metastatic stomach cancer in lung | 1 | 1 |
| Paracancer of stomach adenocarcinoma | 12 | 12 |
| Hepatocellular liver cancer | 34 | 39 |
| Liver cholangiocellular carcinoma | 15 | 16 |
| Liver clear cell carcinoma | 3 | 3 |
| Liver adenosquamous carcinoma | 4 | 4 |
| Liver bile duct adenocarcinoma | 5 | 5 |
| Metastatic liver adenocarcinoma in | 1 | 1 |
| spleen | | |
| Prostate adenocarcinoma | 536 | 540 |
| Paracancer of prostate adenocarcinoma | 5 | 5 |
| Lung squamous cell carcinoma | 30 | 32 |
| Lung adenosquamous carcinoma | 4 | 4 |
| Lung adenocarcinoma | 19 | 20 |
| Lung small cell carcinoma | 4 | 4 |
| Metastatic lung cancer in lymph node | 2 | 2 |
| Metastatic lung cancer in abdominal | 1 | 1 |
| cavity | | |
| Metastatic rectum adenocarcinoma in | 3 | 3 |
| lymph node | | |
| Metastatic epiploon adenocarcinoma in | 2 | 2 |
| lymph node | | |
| Metastatic gallbladder adenocarcinoma | 2 | 2 |
| in lymph node | | |
| Metastatic intestine adenocarcinoma in | 1 | 1 |
| lymph node | | |
| Metastatic jaw adenocarcinoma in | 1 | 1 |
| lymph node | | |
| Thyroid carcinoma | 1 | 1 |
| Metastatic thyroid papillary carcinoma | 2 | 2 |
| in lymph node | | |
| Metastatic thyroid papillary carcinoma | 2 | 2 |
| in cerebrum | | |
| Metastatic ovary adenocarcinoma in | 3 | 3 |
| epiploon | | |
| Adenoid Cystic Carcinoma | 5 | 5 |
| Adrenal Gland Cancer | 23 | 23 |
| Anal cancer | 3 | 3 |
| Appendix cancer | 55 | 55 |
| Basal Cell Carcinoma | 4 | 4 |
| Bile Duct Cancer | 23 | 23 |
| Bladder Cancer | 46 | 46 |
| Bone Cancer | 212 | 212 |
| Brain Tumor | 120 | 124 |
| Carcinoid Tumor | 4 | 4 |
| Cervical Cancer | 53 | 53 |
| Ear cancer | 14 | 14 |
| Endometrial Cancer | 3 | 3 |
| Eye cancer | 4 | 4 |
| Gallbladder cancer | 26 | 26 |
| Esophageal Cancer | 5 | 5 |
| Head and neck cancer | 12 | 12 |
| Intestinal cancer | 15 | 15 |
| Islet Cell Tumor | 4 | 4 |
| Kaposi's sarcoma | 3 | 3 |
| Kidney Cancer | 123 | 123 |
| Laryngeal Cancer | 5 | 5 |
| Liver Cancer | 334 | 334 |
| Lobular Carcinoma | 5 | 5 |
| Lung Cancer | 543 | 543 |
| Lung neuroendocrine tumor | 4 | 4 |
| Malignant Glioma | 46 | 46 |
| Mesothelioma | 5 | 5 |
| Nasal and paranasal sinus cancer | 4 | 4 |
| Nasopharyngeal Cancer | 3 | 3 |
| Neuroblastoma | 121 | 121 |
| Neuroendocrine Tumor | 3 | 3 |
| Oral Cancer | 17 | 17 |
| Oesophageal cancer | 4 | 4 |
| Osteosarcoma | 3 | 3 |
| Ovarian Cancer | 45 | 45 |
| Pancreatic Cancer | 222 | 222 |
| Parathyroid Cancer | 4 | 4 |
| Penile Cancer | 3 | 3 |
| Peritoneal Cancer | 4 | 4 |
| Pituitary Gland Tumor | 5 | 5 |
| Prostate Cancer | 335 | 335 |
| Renal Cell Carcinoma | 5 | 5 |
| Salivary Gland Cancer | 33 | 33 |
| Sarcoma | 14 | 14 |
| Signet cell cancer | 3 | 3 |
| Sinus cancer | 3 | 3 |
| Skin Cancer (Melanoma) | 122 | 122 |
| Soft tissue sarcoma | 57 | 57 |
| Spinal cancer | 4 | 4 |
| Stomach Cancer | 245 | 245 |
| Stomach neuroendocrine tumors | 3 | 3 |
| Testicular Cancer | 24 | 24 |
| Throat cancer | 4 | 4 |
| Thymus gland tumors | 5 | 5 |
| Thyroid Cancer | 17 | 17 |
| Tongue cancer | 23 | 23 |
| Tonsil cancer | 4 | 4 |
| Uterine Cancer | 26 | 26 |
| Unknown primary solid tissue cancer | 3 | 3 |
| Vaginal Cancer | 14 | 14 |
| Vulvar cancer | 13 | 13 |
| Wilms' Tumor | 6 | 6 |

| | |
|---|---|
| Total tested samples | 7107 |

### Example V

### Detection of CTs in Breast Cancer Tissue Using Needle Biopsy

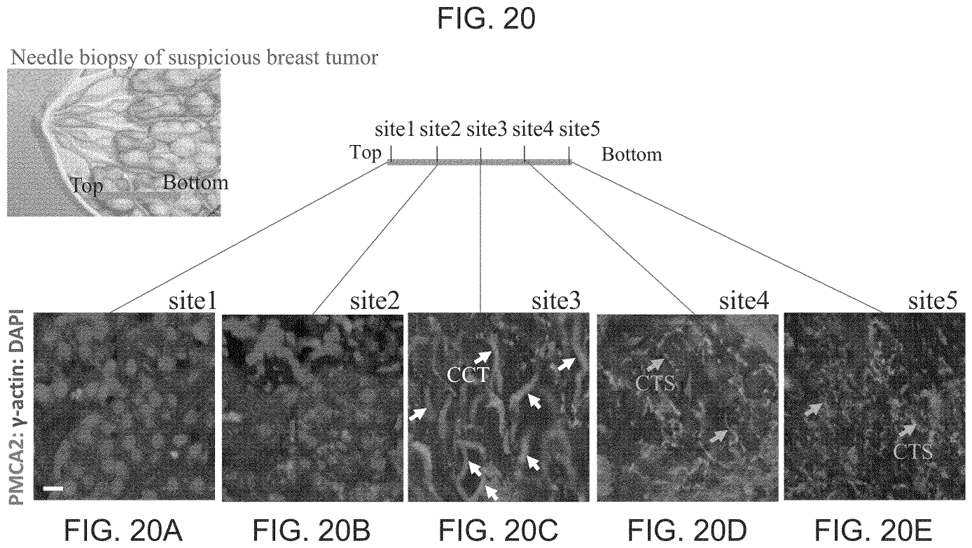

Needle biopsy is a powerful tool for cancer prognosis and diagnosis with minimum tissue damage. Next, needle biopsy samples of clinical breast cancer tissues were examined (5 pieces of specimens from 5 different cancer patients of breast cancer, 1 in stage I, 2 in stage II, 1 in stage III, and 1 in stage IV; 1 piece of specimen per patient, needle biopsy) with the method of IHC-fluorescence assays with antibodies of anti-PMCA2. Scanning from the top end to the bottom end of the needle biopsy sample, the CCTs along the thin and long needle biopsy samples were detected (FIGS. 20A-20E) and obtained CCT numbers, densities (0-112 CCTs/mm² in CCT density) and morphology along the depths of the tissue. These results suggested that the method of IHC-fluorescence assays with antibodies of anti-PMCA2 for detection of CCTs can be applied to clinical needle biopsy samples.

### Example VI

### Detection of CTs Using Anti PMCA1-4 Antibodies

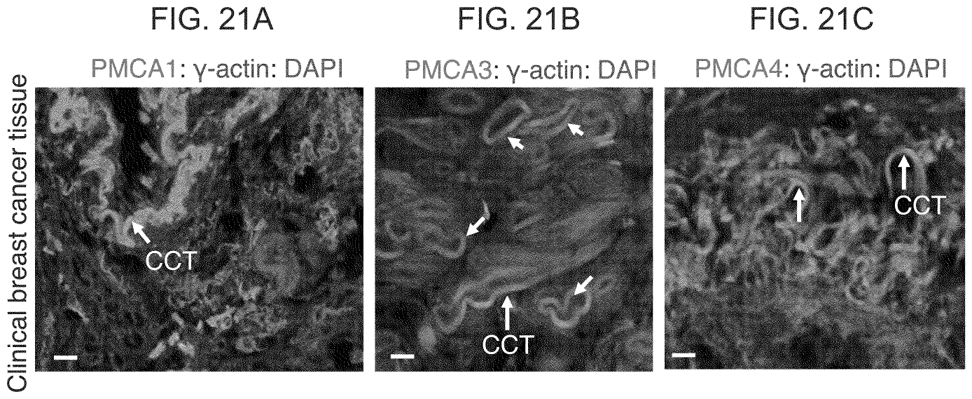

PMCA has four homologs, PMCA1-4. Next, whether other PMCA such as PMCA1, PMCA3, and PMCA4 can be detected in CCT membranes and used for CCT detection in cancer analyses was investigated. We performed IHC-fluorescence assays with antibodies of anti-PMCA1, anti-PMCA3 and anti-PMCA4 using clinical breast cancer tissues (5 pieces of specimens from 5 different cancer patients of breast cancer in stage III; 1 piece of specimen per patient, surgical biopsy). Indeed, antibodies of anti-PMCA1, anti-PMCA3 and anti-PMCA4 showed many CCTs of various densities (35-112 CCTs/mm² in CCT density) in clinical breast cancer tissues and in diverse stages progressing throughout CCT lifecycle (FIGS. 21A-21C). Therefore, these data evidenced that the methods of IHC-fluorescence assays with antibodies of anti-PMCA1, anti-PMCA2, anti-PMCA3 and anti-PMCA4 can be used as various embodiments of the present methods detection of CCTs in all kinds of cancer tissues, such as all solid tumor cancers. The present methods are useful in the cancer prognosis, diagnosis and monitoring in humans and animals.

The membrane proteins of PMCA1, PMCA2, PMCA3 and PMCA4 were highly expressed in membranes in cytocapsular tubes in comparison with their relatively low abundance in the membranes of cancerous cells and normal cells. The ratio of the relative levels of expression were found as follows: about 20:1 for PMCA1 in CCTs vs. cancer/normal cells; about 60:1 for PMCA2 in CTs vs. cancer/normal cells; about 15:1 for PMCA3 in CCTs vs. cancer/normal cells; and about 12:1 for PMCA4 in CCTs vs. cancer/normal cells. The high abundance of PMCAs in CCTs facilitates detection of CCTs with high sensitivity above backgrounds in diverse clinical cancer tissue analyses, which helps to advance IHC-fluorescence assays with antibodies of anti-PMCAs comparing to conventional H&E staining and IHC staining assays with other cancer cell molecular markers.

### Example VII

### Proposing a Cancer Metastasis Grade Classification System Using CCT Analysis

Based on the CCT density and morphology that were observed in their association with cancer tissue stages of all the cancer tissues examined, it was found that CCTs of lower density and little or no degradation are associated with early cancer tissue stages of the tissues examined, while CCTs of higher density and degradation are associated with more advanced cancer tissue stages of the tissues examined. A predictive new classification system of Cancer Metastasis (CM) Grade with grades 0-4 is thus proposed based on the density and morphology/degradation of CCTs in the cancer tissue. For example, if no cytocapsular tube (CCT) is detected and no CCT degradation is detected, cancer metastasis (CM) of grade 0 is predicted; if the density of CCT detected is in the range of 1-10/mm² and no CCT degradation is detected, cancer metastasis (CM) of grade 1 is predicted; if the density of CCT detected is in the range of 11-40/mm², and CCTs are degraded into strands and thin strands, cancer metastasis (CM) of grade 2 is predicted; if the density of CCT detected is in the range of 41-80/mm², and CCTs are degraded into strands, thin strands, and silk-like strands, cancer metastasis (CM) of grade 3 is predicted; and if the density of CCT detected is in the range of >81/mm², and CCTs are degraded into cloud-like morphology or completely decomposed, cancer metastasis (CM) of grade 4 is predicted. The relationships between the proposed CM grade and conventional cancer stages are shown in Table 5.

TABLE 5
A comparison of clinical cancer stages and the
cancer metastasis (CM) grades proposed based on
the described herein in the present disclosure:

| Clinical Cancer Stage | Cancer Metastasis (CM) Grade |
|---|---|
| 0 | 0, 1, 2, 3, 4 |
| I | 1, 2, 3, 4 |
| II | 2, 3, 4 |
| III | 3, 4 |
| IV | 4 |

The conventional cancer diagnosis cannot detect CCTs (which are difficult to find due to their size of about 3-6 μm in diameter/width). For example, by conventional cancer diagnosis, a presumed “normal” tissue at stage 0 may actually contain CCTs of various density and degradation stages, which can be detected by the methods described herein. Therefore, a clinical cancer stage 0 can correspond to CM grades from 0-4. The methods of the present disclosure can predict real healthy tissues if no CCTs is detected in the tissue, thus offering a more sensitive and precise prediction of cancer metastasis grades compared to conventional cancer diagnosis methods.

Cancer metastasis, a feature of malignant tumors, is a major source of cancer lethality, while the mechanisms underlying cancer dissemination has long been obscure (Sporn, M. B. (1996). The war on cancer. Lancet 347, 1377-1381; Kinzler, K. W., and Vogelstein, B. (1998). Landscaping the cancer terrain. Science 280, 1036-1037; Chambers, A. F., and Matrisian, L. M. (1997). Changing views of the role of matrix metalloproteinases in metastasis. J. Natl. Cancer Inst. 89, 1260-1270; Johnson, J. P. (1991). Cell adhesion molecules of the immunoglobu-lin supergene family and their role in malignant transformation and progression to metastatic disease. Cancer Metastasis Rev. 10, 11-22). Embodiments of the present disclosure are based on the surprising discovery that endogenous PMCAs are highly expressed in the membranes that forms the cytocapsular tubes in cancer tissues. Antibodies raised against PMCA, including anti-PMCA1, anti-PMCA2, anti-PMCA3 and anti-PMCA4 antibodies have been shown to detect their respective PMCA1-4 proteins in CCT membranes in cancer tissues using IHC-fluorescence assays. No CCTs were detected in healthy or benign tissues. The data suggested that cancer cells within the cancer tissues generate these membrane-enclosed cytocapsular tubes and use these CCTs as cancer cell metastasis freeways for cancer migration and dissemination. Hundreds of cancer types and subtypes have been investigated by the present method. Cytocapsular tubes were detected in all clinical cancers investigated and images were shown for 32 kinds of representative solid tumor tissues and organs with both surgical and needle biopsy samples. These data evidenced that the present method provide for precise and quantitative analyses of cytocapsular tubes that are useful for precise cancer metastasis degree prediction in cancer prognosis, diagnosis and monitoring during cancer treatment and management of all kinds of human and animal solid tumor cancers, and that methods of IHC-fluorescence assays with antibodies of anti-PMCA1, anti-PMCA2, anti-PMCA3 and anti-PMCA4 can reliably to be used for the detection of cytocapsular tubes in cancers, paracancer tissues and metastatic tissue and organs.

Precise cancer metastasis analyses are previously impossible due to the lack of clear mechanisms underlying cancer metastasis. The data described herein provided strong evidence that CCTs are membrane-enclosed freeways for cancer cell dissemination and migration to neighbor and far distance tissues and organs in all kinds of solid tumor cancers. The present methods pave the way for precise cancer assays during prognosis, diagnosis and monitoring before and after therapy/treatment/management. CCT analysis will be a powerful tool in the precise, quantitative analysis of human cancers and cancer metastasis prediction in clinical cancer patient treatment procedures. CCTs will undoubtedly be the targets for the development of drugs and treatment methods in order to effectively inhibit cancer metastasis since cancer cells depend on CCT to conducted cancer cell dissemination and migration.

Embodiments of the present disclosure also direct to grading cancer metastasis by CCT density and morphology detected in cancer tissues. The proposed Cancer metastasis (CM) grades will facilitate the precise and quantitative analyses of cancer metastasis status, and therefore augment the effectiveness and efficiency of cancer treatment and management. CCTs in cancers are always in complex appearances with curved, entangled, circled, compact, large structures with irregular architectures and shapes, which make it difficult to accurately count the CCT numbers/densities. Therefore, the CCTs numbers and densities can be only an estimation. In addition, CCT with or without degradation and in variant thickness strand morphology are often detected in the same sample tissue and mixed together, which also increase the difficulty to accurately quantity cancer metastasis grades by relying on CCT density and morphology. However, the CCT grades suggested here, which are based on the analyses of >7,000 cancer patients (from 5 countries and America, Asia and Europe continents) covering >260 types and subtypes of human cancers, is a powerful tool for the relatively precise and quantitative cancer analyses as detection methods and statistics improve. Embodiments of the present disclosure are directed to cytocapsular tubes detection by IHC-fluorescence assays with antibodies of anti-PMCAs (PMCA1-4) for the detection of cytocapsular tube density and morphology. The data suggested that CCTs are formed by the cancerous cells in the cancer tissues and undergo various stages of degradation throughout the CCT life cycle. With the appearance of fewer, shorter and thicker cytocapsular tubes at the initial stage and the CCTs grown to longer, larger and thinner tubes, CCTs then degrade to thinner strands and cloud-like morphologies in later stages of degradation and finally decompose completely.

Further, the so called normal adjacent tissues (NAT) have been found to associate with a large quantity of CCTs in CCT analysis. These findings will help to correct the false “NAT” as abnormal paracancer tissues, and therefore help clinical doctors to obtain samples with improved precision for correct diagnosis, prognosis and evaluation of cancer, and choose optimal treatment protocols for cancer patients, reducing cancer recurrence due to cancer spreading from previous “normal adjacent tissues”.

### Example VIII

### Materials and Methods

### Reagents and Antibodies.

Rabbit polyclonal antibodies of anti-PMCA1-4 (1:200 dilution) were produced by immunizing rabbits with each of the PMCA1-4 antigens. DAPI (1:1,000 dilution in the immunofluorescence assay) was ordered from Sera Care. The following antibodies (all in 1:1,000 dilution) were ordered from Abcam: for the immunofluorescence assay, anti-γ-actin (γ-actin, monoclonal, ab123034).

### H&E Staining Assay.

The clinical Formalin-Fixed Paraffin-Embedded (FFPE) tissue (normal tissue, benign tumor tissue and cancer tissues) specimens were ordered from US Biomax, Inc. or obtained from hospitals in Boston, Chicago and Florida of USA. The H&E staining assays were performed as previously described. (Barbolina M V, et al. (2009) Microenvironmental regulation of ovarian cancer metastasis. Cancer Treat Res 149:319-334.)

### Immunohistochemistry (IHC)-Fluorescence Assay.

The clinical FFPE tissue (normal tissue, benign tumor tissue and cancer tissues) specimens were ordered from US Biomax, Inc. or obtained from hospitals of USA, Ukraine, Vietnam, and China.

Deparaffinization and rehydration of tissue slides a retrieval of antigen (0.6 mM sodium citrate, boiling for 30 min) a Cool down for 30 min 0.3% H2O2 treatment for 15 min 1% BSA in 1×TBST buffer blocking for 30 min, followed by primary antibody against PMCA2 (1:200 dilution) incubation in dark room for 40 min, followed by 3 washes with TBST buffer. Secondary goat, anti-rabbit IgG Alexa flour 488 (green color) antibodies (1:1000 dilution) were added and incubation for 30 min, followed by 3 washes with TBST buffer, 1% FBS in 1×TBST buffer blocking for 30 min. Primary anti-gamma-actin antibodies (1:200 dilution) incubation in dark room for 40 min, followed by secondary goat, anti-mouse IgG Alexa Fluor 568 (red color) antibodies (1:1000 dilution) incubation for 30 min, followed by 3 washes with TBST buffer DAPI staining (1:1000 dilution) for 5 min. Then mount immunofluoresence stained slides. Imaging was done with a fluoresence microscope and analysis of breast cancer cell cytocapsular tubes for breast cancer metastasis identification and evaluation was done.

### Quantification and Statistical Analysis

The diameters, widths, and lengths, numbers and densities of cytocapsular tubes (CCTs) were measured with ImageJ. For the clinical surgical biopsy specimens, the CCT numbers of the 5 sites (left, top, right, bottom, and center sites) were counted, and the CCT densities (CT/mm²) were calculated. The average CCT density of the 5 sites was used as CCT density of a piece of specimen.

### Example IX

### Identification of PMCA2 as a Biomarker for Cytocapsulas and Cytocapsular Tubes

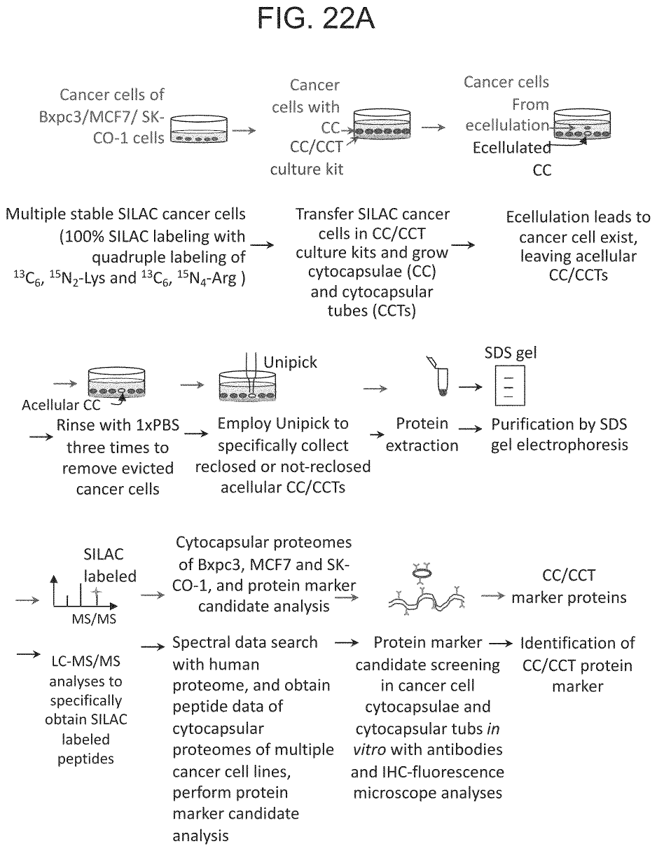

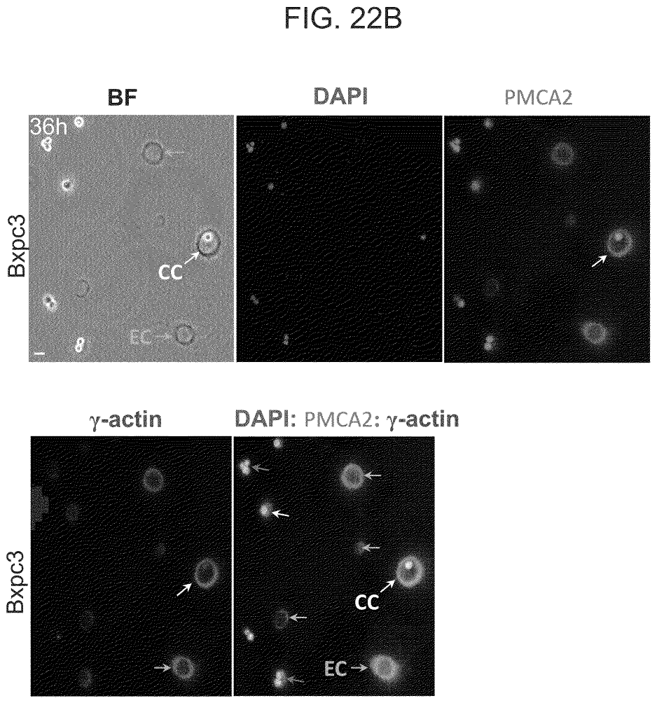

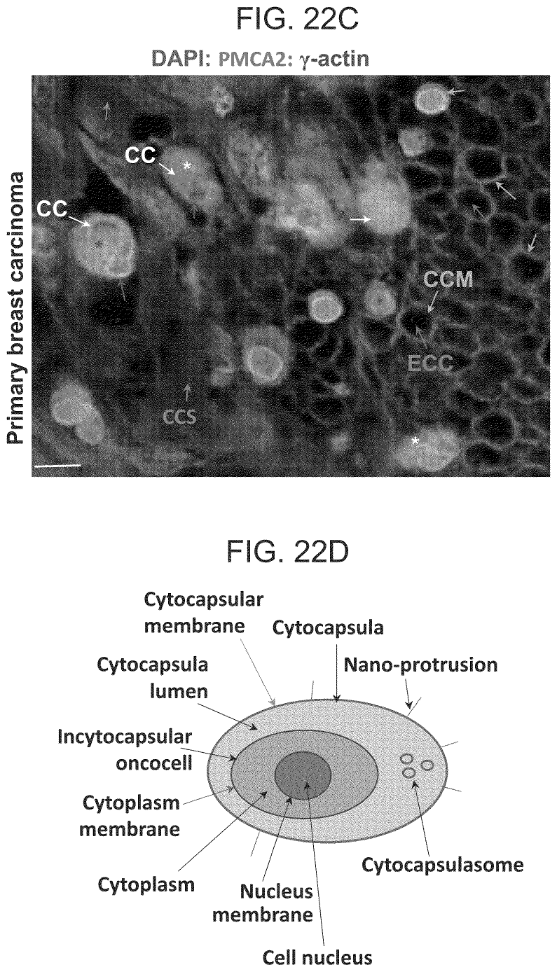

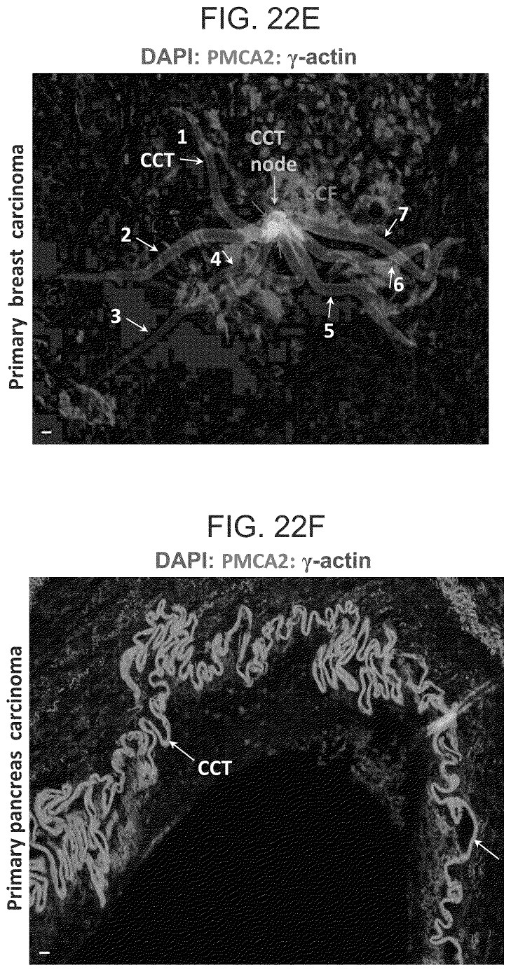

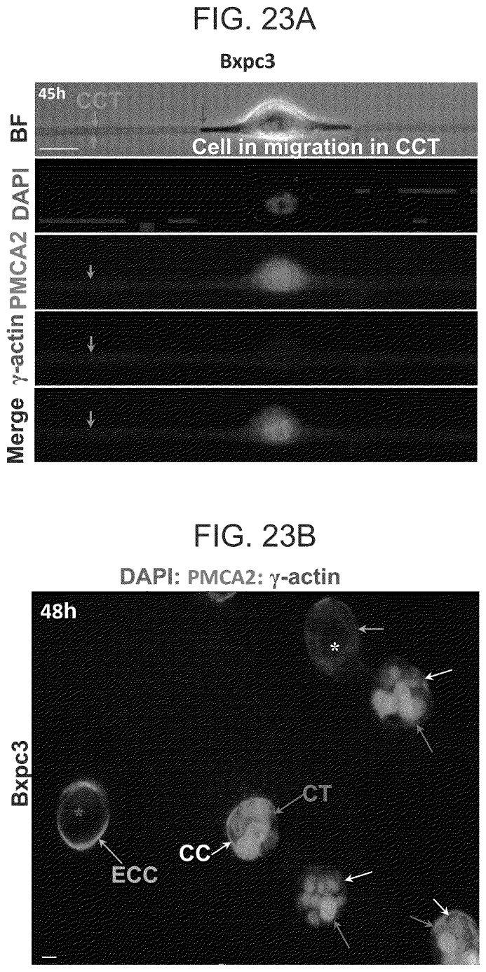

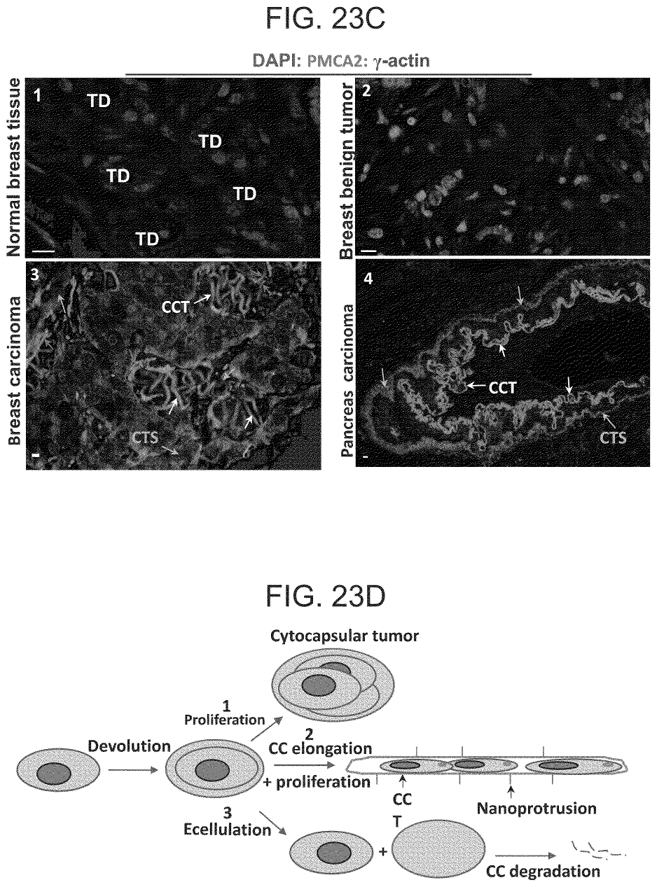

To explore the structure and protein components of the previously discovered cytocapsula (CC) membranes, we developed conditions for ecellulation of cancer cells from the cytocapsulas in in vitro 3D matrix culture, in order to obtain the proteome of the acellular cytocapsulas. Using CC/CCT 3D matrix culture kits and Unipick, we cultured and collected acellular SILAC labelled cytocapsulas (CCs) of Bxpc3 pancreas cancer cells, MCF-7 breast cancer cells, and SK-CO-1 colon cancer cells for CC proteome assays (FIG. 22A). A prominent protein was found to be the calcium pump plasma membrane Ca²⁺-ATPase 2 (PMCA2) that consistently appeared in the plasma membranes of the enclosed cancer cells (FIG. 22B). However, PMCA2 was found to be at much higher abundance in the CC membranes encapsulating single or multiple cancer cells (number tested, n=601), or in ecellulated CC membranes (n=514) in vitro. No PMCA2 signal was found in the CC/CCT culture kit matrix outside the CCs or CCTs (FIG. 22B). PMCA2 and γ-actin were found to be constantly colocalized in cancer cell plasma membranes (n=601) and in completely ecellulated CC membranes (n=514) in vitro (FIG. 22B). Consistently, PMCA2 showed high abundance and colocalized with γ-actin in the cytocapsular tube (CCT) membranes surrounding Bxpc3 cancer cell (n=106, FIG. 23A). The same was found in the enlarged CC membranes surrounding tumorspheres (n=546, FIG. 23B), and in the ecellulated enlarged CC membranes of tumorspheres (n=127, FIG. 23B). These observations suggested that PMCA2 is a molecular biomarker of CCs and CCTs in vitro. Next, we analyzed human-normal, benign-tumor and malignant-tumor (cancer) tissues with anti-PMCA2 antibodies. In clinical normal patients (n=14 patients, 1 tissue/patient) (FIG. 23C, panel 1) or benign-tumor tissues (n=126 patients, 1 tissue/patient), (FIG. 23C, panel 2, there were very low PMCA2 signals and no CC/CCTs, indicating that normal tissues and benign-tumor tissues don't generate CCs/CCTs, and that PMCA2 expression is tightly controlled and maintained at low abundance in human normal tissues or benign tumors (FIG. 23C, panels 1 and 2). In contrast, in clinical breast (n=685 patients, FIG. 23C, panel 3) and pancreas (n=310 patients, FIG. 23C, panel 4) carcinoma tissues, there were many long and curved CCTs with high abundance of PMCA2 in the CCT membranes (FIG. 23C, panels 3 and 4). In addition, there were no PMCA2 signals in the extracellular matrix (ECM) in normal, benign and cancer tissues in vivo (FIG. 23C). The above observations suggest that PMCA2 is a molecular marker of CCs/CCTs in vitro and in vivo.

### Example X

### Characterization and Lifecycle of Cytocapsular Oncocells In Vitro and in Human Cancer Tissues In Vivo

With a cytocapsula (CC)/cytocapsular tube (CCT) biomarker PMCA2 in hand, we tested whether cancerous cells generate CCs in vivo. Indeed, in the early stage of breast carcinoma, it was found that there are many cancerous breast cells enclosed by CCs, which shield cancerous cells inside, and isolate and protect them from the extra-cytocapsular microenvironments (FIG. 22C). CCs display diversities in morphologies, sizes, taut or folded membranes, and with/without ecellulation, autodegradation and autodecomposition. Some CCs (n=135) appear in irregular morphologies with highly folded membranes wrapping breast cancer cells, while others (n=268) tightly and smoothly wrap around breast cancer cells (FIG. 22C). Some breast cancer cells ecellulate from the CCs (n=1023), leaving acellular CCs behind (FIG. 22C, ECC). These acellular CCs align together and form acellular CC groups (FIG. 22C). Some acellular CCs appear in round or oval morphologies (n=576), and many adopt irregular shapes (n=1217) (FIG. 22C). Some acellular CC membranes indicate a taut and stringent status (n=107, FIG. 22C). The CC ecellulation phenomena and acellular CCs' morphologies in vivo (FIG. 22C) are consistent with those in vitro (FIG. 22B and FIG. 23B). Importantly, cancer cells proliferate in CC lumens and migrate in CCT lumens in vitro and in vivo (FIGS. 22B-22C and FIGS. 23A-23B). These observations suggest that the cytocapsular membranes not only shelter and protect cancerous cells inside but also allow cancerous cells to execute cellular behaviors and activities in the CC/CCT lumens. We termed this previously unrecognized single cancerous cell that is enclosed in an extracellular second membrane of the CC, or CCT a “cytocapsular oncocell” (FIG. 22D). It performs cellular activities in the cytocapsular lumen.

Next, we investigated individual cytocapsular oncocell activities and behaviors in vitro and in human cancer tissues in vivo. It was found that single cytocapsular oncocells generate long CCTs wherein they migrate in vitro. CCT membranes tightly wrap oncocell and display bulges (FIG. 23A). The stretched and contracted CCTs are 2~3 μm in width/diameter, while the bulged CCT fragments enlarged by oncocell inside are 5~10 μm in width, increasing 2.5~5-folds in width and 7.85~15.7-fold in calculated perimeter (perimeter=3.14×diameter in a circle). These observations suggest that CCT membranes are propertied with potent elasticity, permitting oncocells in variable sizes to dynamically migrate inside (FIG. 23A). A single breast cytocapsular oncocell can aggressively engender multiple CCTs in different directions but connected by a node in compact breast carcinoma in vivo (FIG. 22E). A single pancreas cytocapsular oncocell can intensely generate a very long and highly curved CCT in pancreas carcinoma tissues (FIG. 22F). These observations strongly suggest that individual cytocapsular oncocells have capacities to engender multiple, long, elastic, and robust CCTs for CCT membrane enclosed freeway-protected and directed cell migration. Cytocapsular oncocells proliferate in cytocapsular lumens and grow into cytocapsular tumorspheres in vitro (FIG. 22B and FIG. 23B) and initiate tumor formation in vivo (FIG. 22C). Sometimes, cytocapsular oncocell ecellulation generates acytocapsular oncocells and acellular cytocapsulas followed by autodegradation into cytocapsular strands (FIG. 22C). In short, the lifecycle of cytocapsular oncocells includes 3 successive procedures: 1) Incytocapsular oncocells proliferate and grow into cytocapsular tumors wrapped in enlarged cytocapsulas, 2) cytocapsulas elongate and generate cytocapsular tubes with oncocells migrating inside, 3) cytocapsular ecellulation engenders acytocapsular oncocells and acellular cytocapsulas (CCs), followed by CC degradation (FIGS. 1A-1F and FIG. 23D).

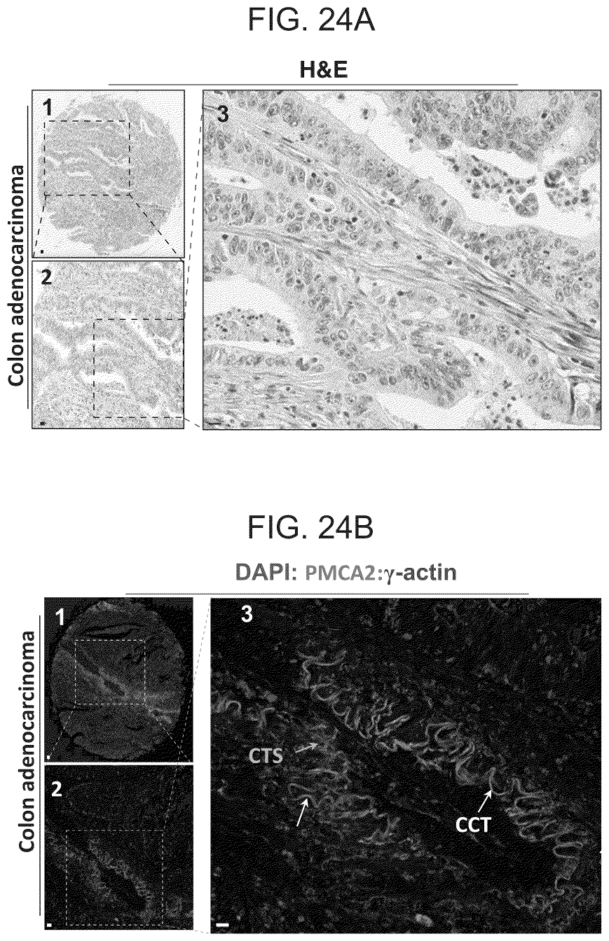

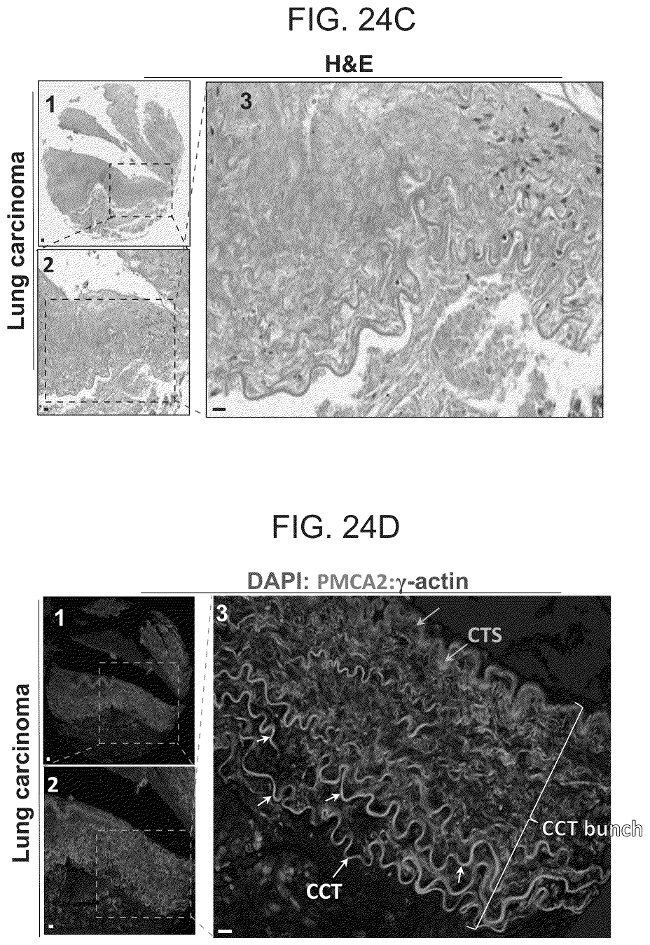

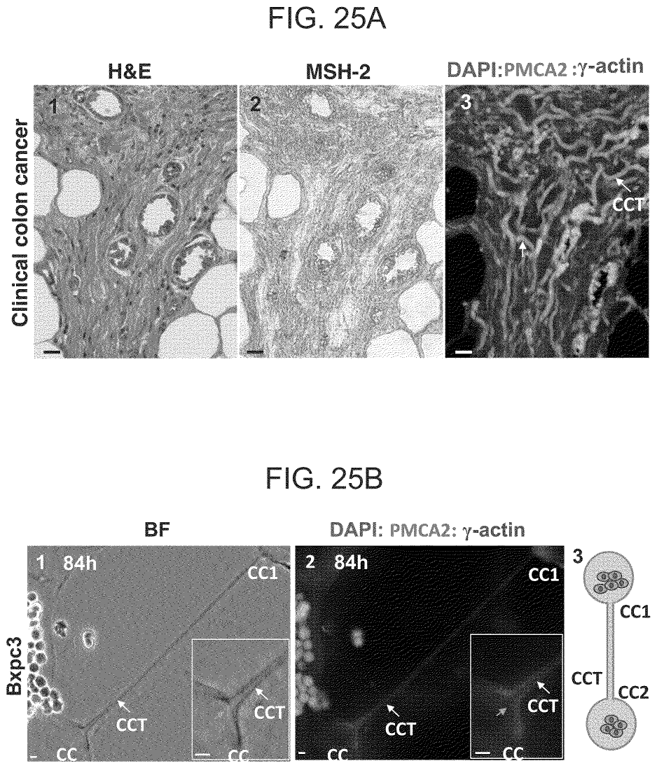

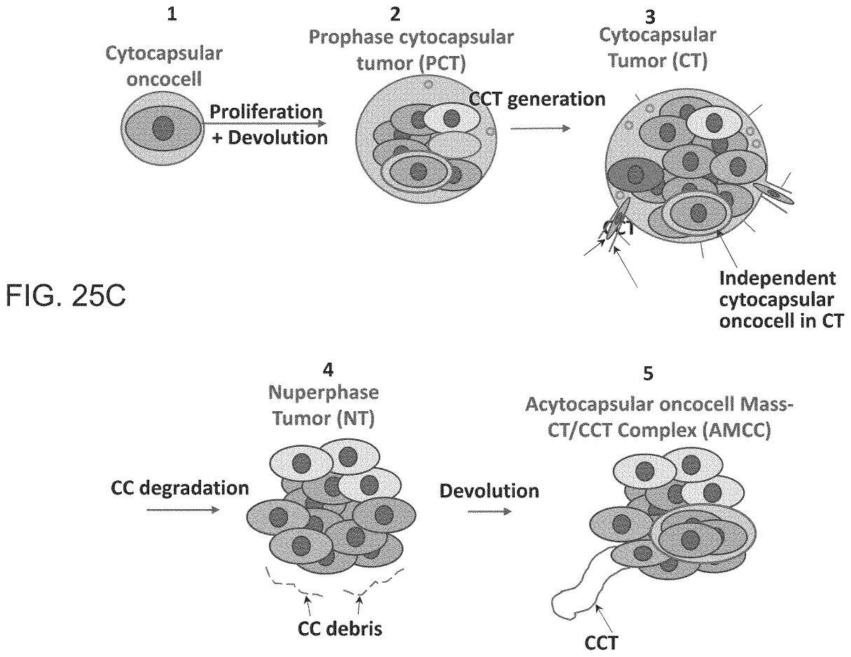

Next, we examined why CCs and CCTs were previously not recognized with conventional methods. In the continuously sectioned adjacent specimens from the same site of the same cancer samples, Hematoxylin and Eosin (H&E) staining does not show features for the large quantities of CCTs in cancers due to the poor staining of CCT membranes by Eosin (n=352 patients, FIG. 24A and FIG. 24C). The antibodies recognizing clinical breast cancer cell marker proteins ER, PR and HER-2 don't recognize CCT marker proteins in these breast cancer samples (n=213 patients, FIGS. 4A-4E). Furthermore, colon cancer cell markers MSH-2 do not show features of CCTs in colon cancers (n=86 patients, FIG. 25A). Indeed, CCs and CCTs could only be detected after we observed the generation of the second membrane outside the cell membrane in CC/CCT 3D matrix culture kits, managed to separate the second membranes from cancer cells, obtained the CC proteome, and compared the relative abundances of the marker from ~10,000 clinically annotated cancer/normal specimens (FIGS. 22A-22F, FIGS. 24A-24D, FIGS. 4A-4E, FIG. 25A).

### Example XI

### Progression and Lifecycle of Cytocapsular Tumors

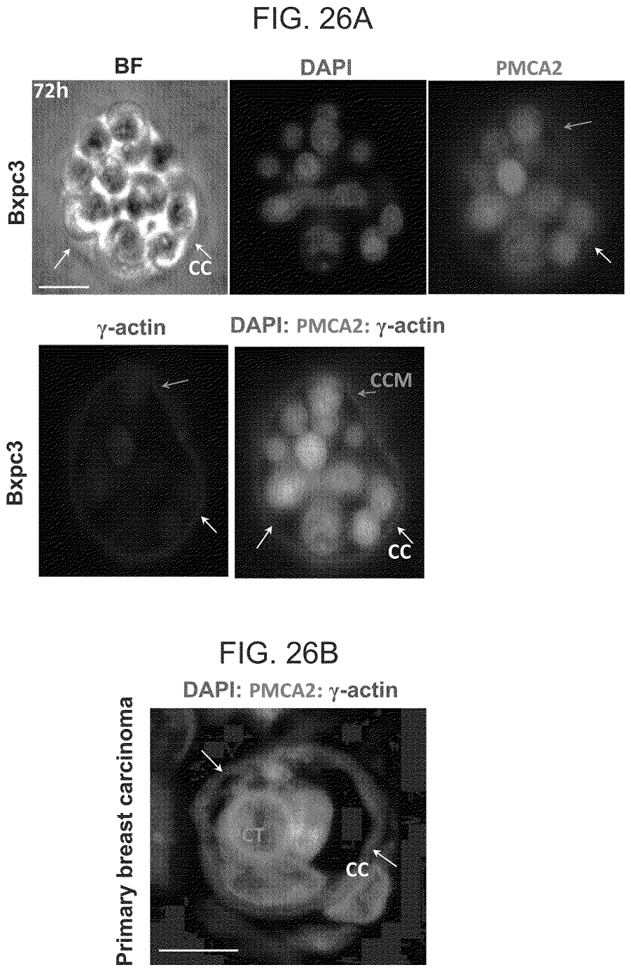

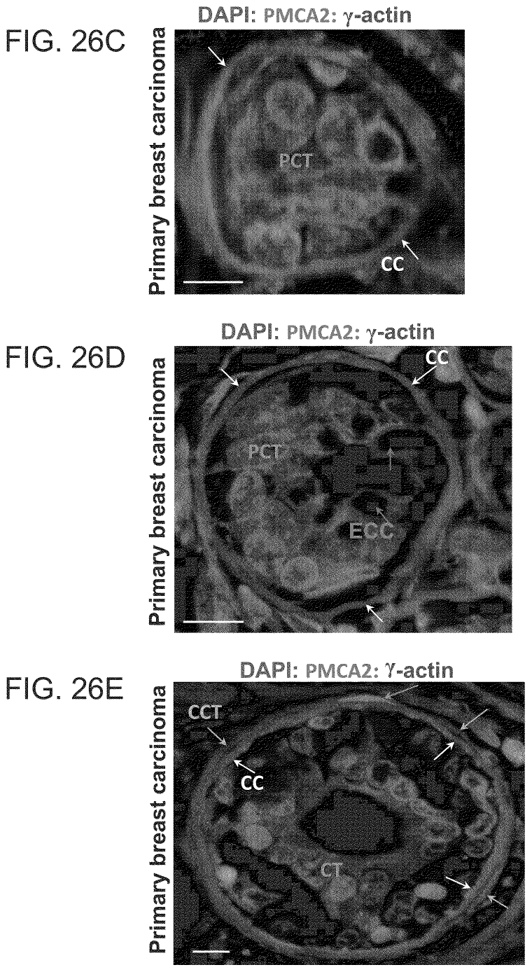

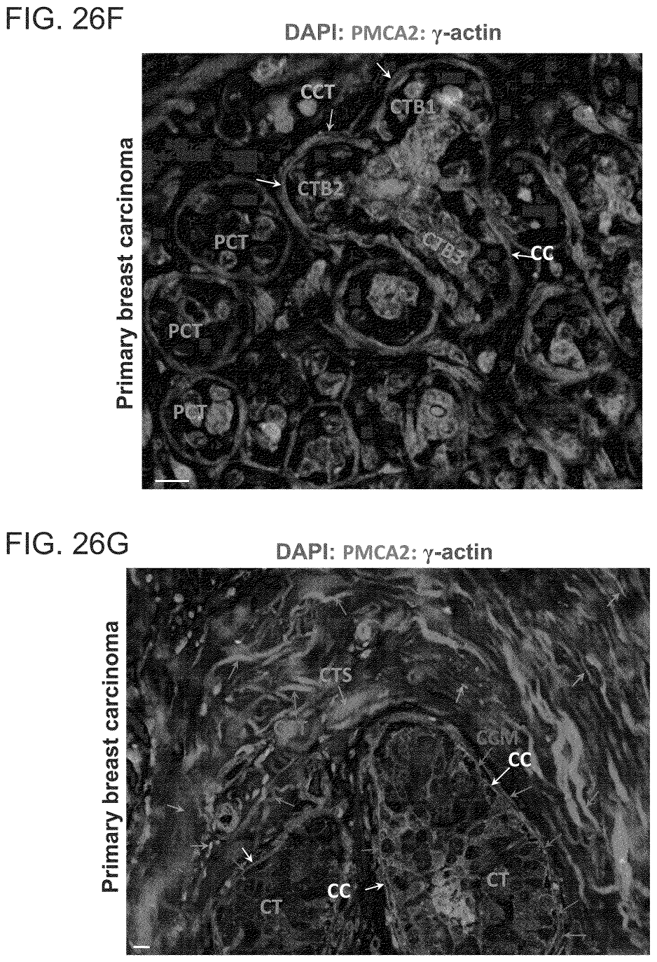

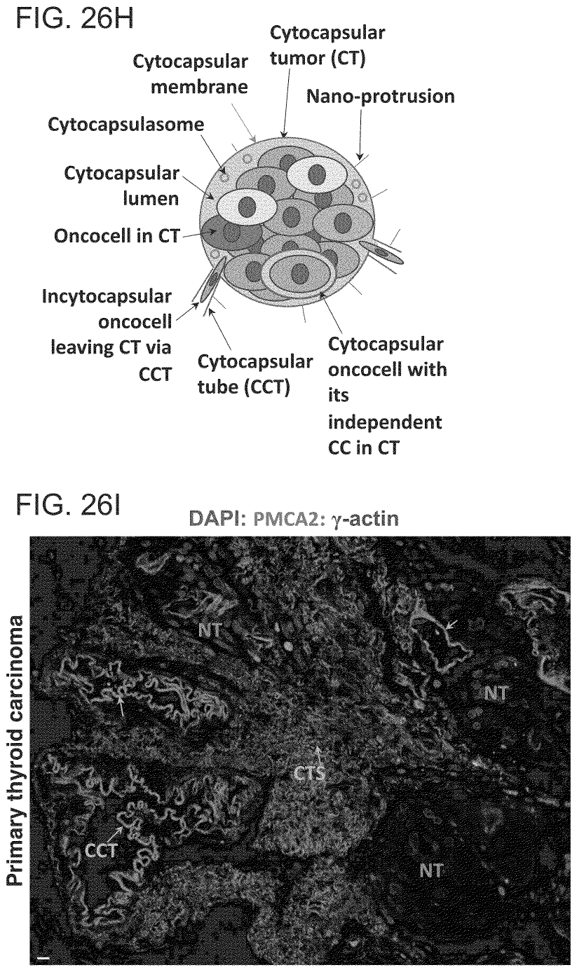

Next, we investigated whether and how individual cytocapsular oncocells grow into tumors. Indeed, at 48 h and 72 h, in the CC/CCT culture kit matrix, cytocapsular Bxpc3 oncocells proliferate in CCs and grow into tumorspheres in vitro, and small CCs develop into enlarged CCs with increased sizes in volume, enclosing big tumorspheres inside (n=458, FIG. 26A and FIG. 23B). The width/diameter of CCs can increase from 8~10 μm of single cytocapsular oncocells to up to 40 μm of big tumorspheres in vitro in our hands, increasing up to 5-folds in width/diameter. The calculated CC volume (v=4/3 πr³ in sphere) of big cytocapsular tumorspheres can be up to 49-folds of that of single cytocapsular oncocell CCs in vitro. These observations suggested that incytocapsular oncocells in CCs have potent capacities to support/drive small CCs to grow into big CCs with enlarged volumes to allow oncocell proliferation and grow into big tumorspheres (FIG. 26A and FIG. 23B). Similarly, like single cytocapsular oncocell ecellulation, some tumorspheres in large CCs perform spontaneous ecellulation (n=23 tumorspheres, FIG. 23B). Individual oncocell ecellulation of tumorspheres in enlarged CCs generates large, reclosed and reunited, acellular, deflated and concave CC discs (n=35, FIG. 23B). Ecellulation of tumorspheres in large CCs engender large, not-reclosed/not-reunited, acellular, deflated CCs with big open holes (n=74, FIG. 23B). These observations evidence that solid cytocapsular oncocells grow into cytocapsular tumorspheres in enlarged CCs in vitro.

Subsequently, we investigated how cytocapsular oncocells progress into malignant tumors in human tissues in vivo. Initially, in the early clinical breast cancer tissues, single cytocapsular oncocells' extracellular cytocapsulas grow slightly larger than 15 μm in diameter/width. Single breast incytocapsular oncocells proliferate and form small oncocell masses composed of several oncocells (n>2 oncocells) in the CC lumens (n=675, FIG. 26B). There are no CCTs emerging from these small oncocell masses at this stage (n=675, FIG. 26B). We name this early stage a “prophase cytocapsular tumor (PCT)” (FIG. 26B). Subsequently, CCs of PCTs grow up and increase in diameter/width, breast incytocapsular oncocells continue to proliferate and form bigger and compacter breast oncocell masses enclosed in the enlarged CCs (n=312, FIG. 26C). The width/diameters of PCTs in the checked breast carcinoma (n=25) are in a range from 15 μm to 50 μm, and the calculated PCTs' CC sizes (estimated in sphere morphology) are in a range from 1,432.7 μm³ to 49,062.5 μm³ in volume (n=89, FIGS. 26B-26D). Sometimes, acellular cytocapsulas (n=161) appear in breast PCT lumens (FIG. 26D), indicating that PCT incytocapsular oncocells generate secondary independent cytocapsulas, and these secondary cytocapsular oncocells can perform ecellulation and create ecellulated cytocapsulas. Subsequently, these ecellulated cytocapsulas in PCTs autodegrade into thin strands, followed by decomposition and disappearance, leaving liquid filled empty cavities (n=78, FIG. 26D). Breast oncocells in the enlarged CCs can extend parts of CC membranes, increase CC membranes in area, deform CC membranes, and form tube-shaped CCTs providing membrane-sheltered freeways for incytocapsular oncocell dissemination outside of CCs of the compact tumors. These CCT membranes are the extensions of CC membranes of PCTs in vitro (n=65, FIG. 25B) and in vivo (n=117, FIG. 26E). We termed this previously unrecognized single malignant tumor, which is wrapped in an enlarged CC and generates CCTs extending into neighbor and far-distance tissues for CCT membrane sheltered and protected oncocell metastasis, a “cytocapsular tumor, CT” (FIG. 26E).

Subsequently, uncontrolled breast incytocapsular-oncocell proliferation produces more oncocells, forms tube-like structures in cytocapsular tumor (CT) lumens, and grows into bigger malignant tumors in the enlarged CC lumens (n=117, FIG. 26E). Frequently, at the early stage, two or more adjacent small breast PCTs, CTs, or PCTs and CTs merge into larger PCTs (or CTs) via CC membrane contact, integration, degradation, and open connection formation, and form long or irregular-shaped cytocapsular tumorspheres enclosed in longer and bigger CCs (n=145, FIG. 26F). Some newly merged CTs have three or more tumor branches (n=23, FIG. 26F). PCTs with quite variable sizes coexist, reflecting heterogeneity of PCTs (FIG. 26F). Later, with uncontrolled oncocell proliferation, the merged CTs with tumor branches remodel and transit into spherical, oval or irregular-shaped and compact CTs (n=132, FIG. 26G). CTs in the examined breast carcinoma (n=51) are in a wide range from 50 μm to 400 μm in width, indicating CC membranes can shelter and protect enlarged CTs until in large sizes. Subsequently, breast CTs generate large quantities of CCTs outside CTs primed for oncocell metastasis. Many long CCTs surround cytocapsular tumors (CTs) and form thick CCT layers enveloping CTs, and the measured thicknesses of CCT layers of CTs are 22~510 μm (n=132 CTs, FIG. 26G). CTs in primary niches interconnect by dense CCT networks and form cytocapsular tumor-network system (CTNS, FIG. 26G). CTs display large heterogeneities in regular or irregular morphologies, sizes, CCT number, thickness of wrapped CCT layers, oncocell density in CC lumens (FIG. 26G). The above observations suggest that cytocapsular tumors with incytocapsular oncocell masses and CCTs (FIG. 26H) provide two inherent physical and structural drivers for the two prime features of malignant tumors (cancers) in clinical observations: uncontrolled proliferation and metastasis (FIGS. 26G-26H).

Subsequently, in the late cancer stage, the CC membranes of CTs degrade, decompose and disappear, leaving compact acytocapsular oncocell masses without CCs (n=814, FIG. 26I). We named this stage of tumor a “nuperphase tumor” (“nuper”, means “late” in Latin). It is composed of acytocapsular oncocell masses after CC degradation without enlarged CCs wrapping the tumor. Some acytocapsular oncocells in nuperphase tumors (NTs) regenerate many new, long, and highly curved CCTs (n=1021NTs). NT oncocells can invade into these CCTs via alloentry and disseminate, leaving decreased oncocell density in place (n=25, FIG. 26I). NT acytocapsular oncocell masses and the newly generated CCTs form a big acytocapsular oncocell mass-CT/CCT complex (AMCC) (n=825, FIG. 26I). In summary, the lifecycle of cytocapsular tumors includes 4 successive procedures: 1) single cytocapsular oncocells generation of prophase cytocapsular tumors (PCTs) without CCTs, 2) generation of cytocapsular tumors (CTs) with many CCTs for cancer metastasis, 3) CC degradation engenders nuperphase tumors (NTs) without CC enclosing oncocell masses, 4) some acytocapsular oncocells in NT regenerate new CCs/CCTs or new small CTs and form AMCCs (FIG. 25C).

### Example XII

### Distribution of Cytocapsular Oncocells and Tumors in Human Tissues and Organs

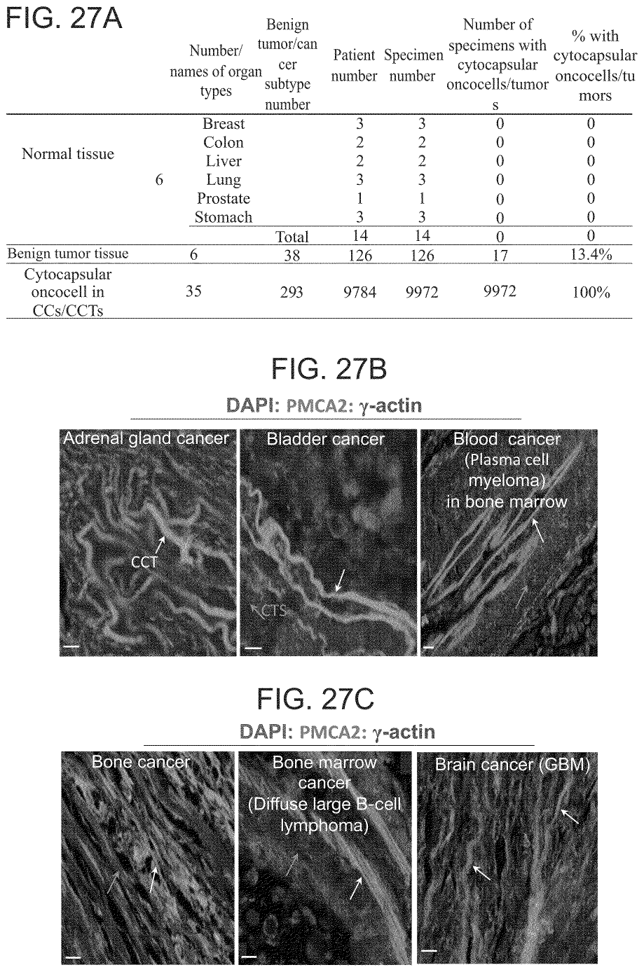

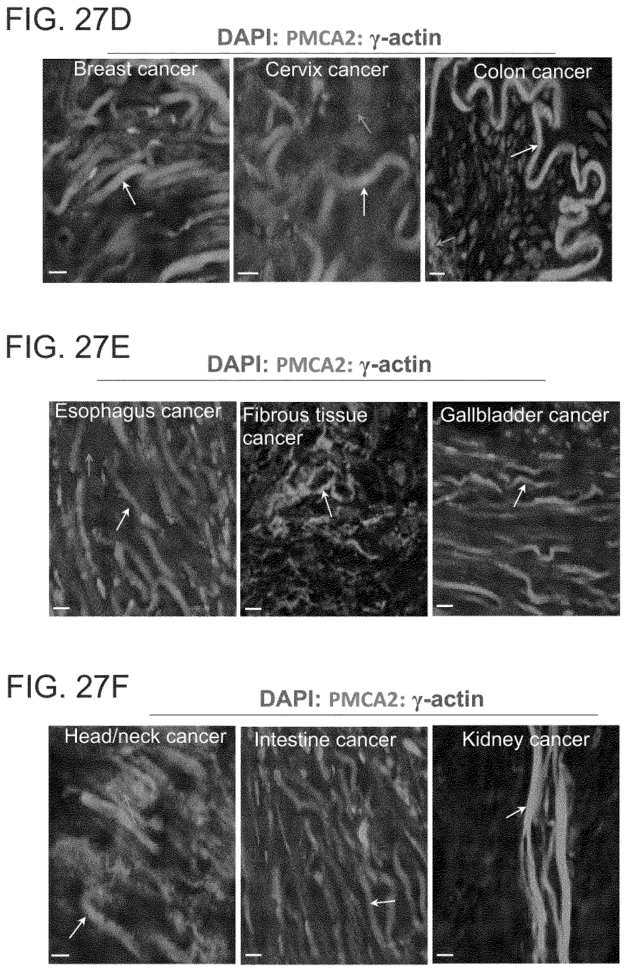

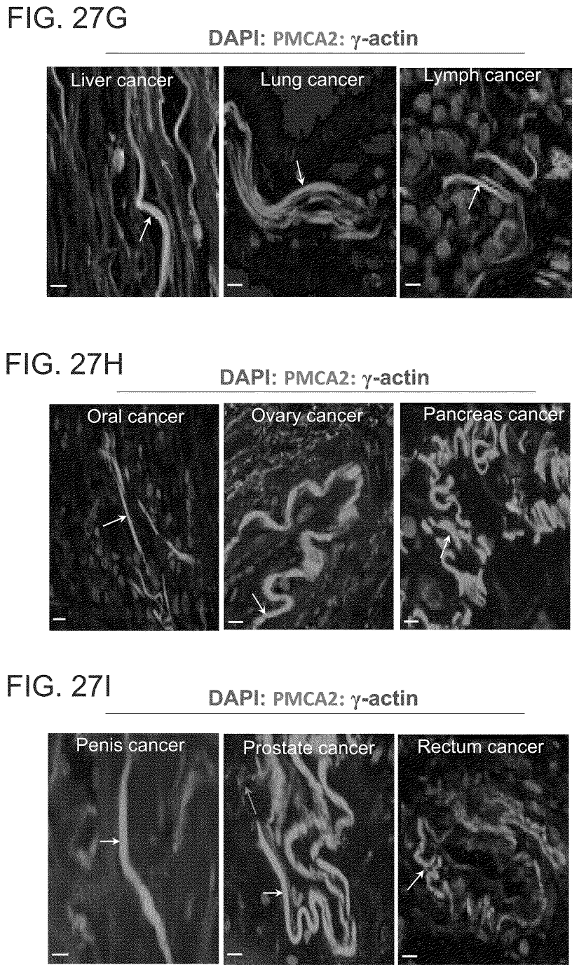

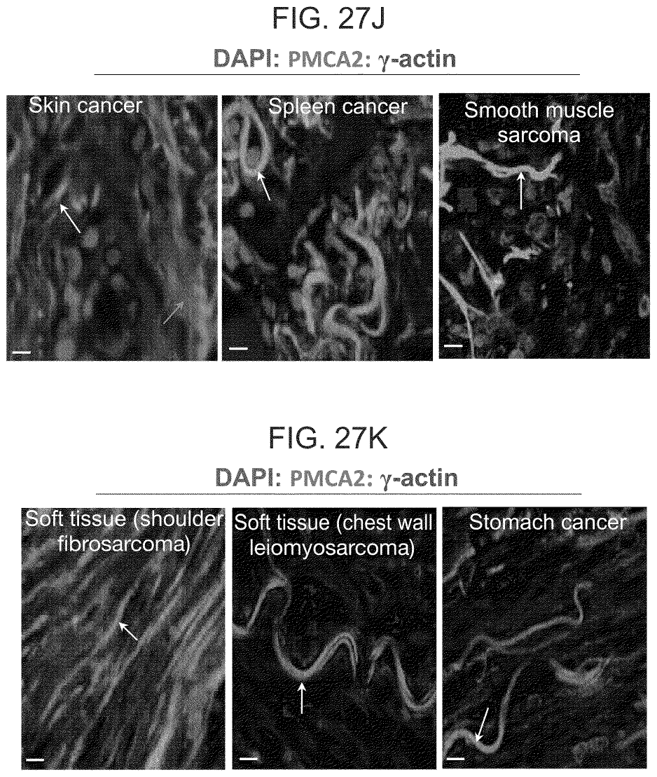

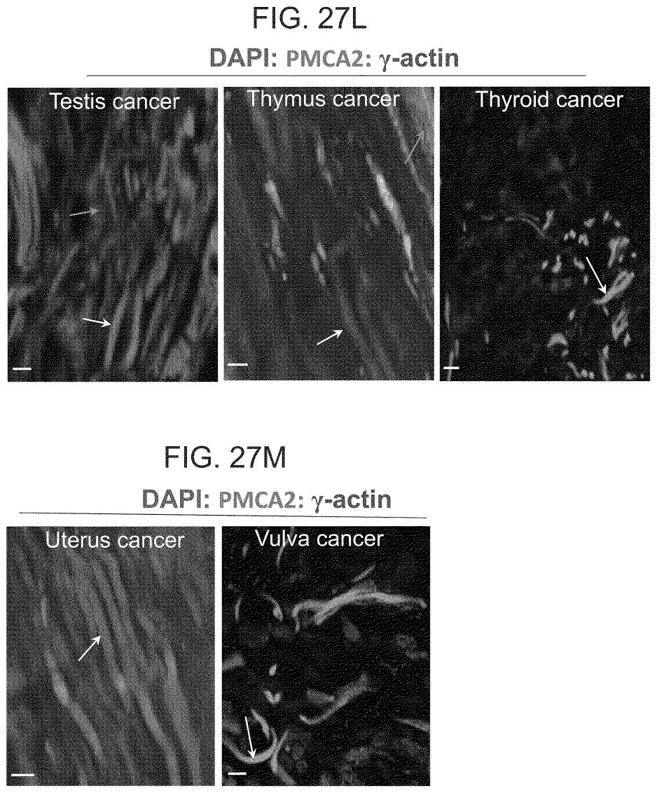

It was well known that cancers universally occur in most human organs and tissues (Sung H, et al., Global cancer statistics 2020: GLOBOCAN estimates of incidence and mortality worldwide for 36 cancers in 185 countries. CA Cancer J Clin. Online ahead of print (2021)). Using PMCA2 as a CC/CCT biomarker, we investigated the distribution of cytocapsular oncocells and tumors in 34 kinds of human organs and tissues. In the examined 6 kinds of normal human organ tissues from breast, colon, liver, lung, prostate and stomach, we did not find detectable amounts of cytocapsular oncocells/tumors (FIG. 27A, n=14 patients). In the tested 38 subtypes of benign tumors (FIG. 27A, n=126 patients), most benign tumors did not exhibit cytocapsular oncocells/tumors, while 13.4% of benign tumors displayed cytocapsular oncocells, indicating the transformation and cytocapsular oncocell occurrence in these tumors clinically annotated as benign. In the tested 290 types/subtypes of cancers of 34 kinds of human organs/tissues (except hematologic cancers in blood, patient number, n=9,770; specimen number, n=9,958), 100% of tested cancers show cytocapsular oncocells (FIG. 27A, and Table 6). These observations suggest that human normal and benign tumor tissues do not generate cytocapsular oncocells, and that human cancers universally engender cytocapsular oncocells. Hematologic cytocapsular oncocells appear in immune organs/tissues of bone marrow, lymph node, spleen and thymus (FIGS. 27B-27M and Table 6). There are many acytocapsular oncocells localized beyond CCs and CCTs, suggesting that acytocapsular oncocells coexist with cytocapsular oncocells in vivo. Cytocapsular oncocells exhibit 3 prominent features: 1) high abundance of PMCA2 in CC/CCT membranes, 2) CCTs are 3-10 μm in diameter/width and up to >3000 μm in length in the sectioned specimens, and 3) localize in CCs or migrate in CCTs (FIGS. 27B-27M and Table 6). Cytocapsular oncocells' CCTs in 283 subtypes of tested solid cancers display vast diversities. These include CCT density, morphologies, super-structures, degradation, interconnections, incytocapsular oncocell migration in CCTs, bunches, mixture with acytocapsular oncocells, which reflects the considerable heterogeneity of cytocapsular oncocells in solid cancers (FIGS. 27B-27M and Table 6). Cytocapsular tumors universally appear in human solid cancers, but not in hematologic cancers in the blood (Table 6). The above observations suggest that cytocapsular oncocells are universally distributed in solid cancers and in hematologic cancers in bone marrow, lymph node, spleen and thymus, but not in normal or benign tumor tissues.

Table 6. Characterization of oncocell, acytocapsular oncocell, cytocapsular oncocell, cytocapsular tumor, and cytocapsular tumor network system in 293 types/subtypes of cancers in human organs and tissues.

| | | | | | | | | | | With | | With | With | |
|---|---|---|---|---|---|---|---|---|---|---|---|---|---|---|
| | | | | | | | | | | cytocapsular | With | cytocapsular | CCT | With |
| | Cancer | | | | | | | | With | oncocell | CCTs | tumors | network | CTNS |
| | types by | | Tissue | | | | | | acyto- | in each | in each | in each | in each | in each |
| | organs/ | *Cancer | specimen | Patient | | | | Cancer | capsular | checked | checked | checked | checked | checked |
| | tissues | subtypes | number | number | Male | Female | Age | stages | oncocell | subtype | subtype | subtype | subtype | subtype |
| 1 | Adrenal | 3 | 23 | 22 | 10 | 12 | 26-71 | I-IV | + | + | + | + | + | + |
| | Gland | | | | | | | | | | | | | |
| 2 | Bladder | 7 | 46 | 46 | 30 | 16 | 36-74 | I-IV | + | + | + | + | + | + |
| 3 | Blood | 3 | 14 | 14 | 8 | 6 | 21-45 | I-IV | + | | | | | |
| | (in blood | | | | | | | | | | | | | |
| | vessel) | | | | | | | | | | | | | |
| 4 | Blood | 2 | 35 | 35 | 18 | 17 | 35-70 | I-IV | + | + | + | | + | |
| | (in bone | | | | | | | | | | | | | |
| | marrow) | | | | | | | | | | | | | |
| 5 | Bone | 12 | 212 | 210 | 115 | 95 | 21-78 | I-IV | + | + | + | + | + | + |
| 6 | Bone | 5 | 20 | 20 | 10 | 10 | 25-64 | I-IV | + | + | + | | + | |
| | marrow | | | | | | | | | | | | | |
| 7 | Brain | 00 | 124 | 122 | 36 | 36 | 24-67 | I-IV | + | + | + | + | + | + |
| 8 | Breast | | | | | | | In situ | - | + | + | + | + | + |
| | | | | | | | | I | + | + | + | + | + | + |
| | | 49 | 3960 | 3890 | | 3890 | 18-86 | II | + | + | + | + | + | + |
| | | | | | | | | II | + | + | + | + | + | + |
| | | | | | | | | IV | + | + | + | + | + | + |
| 9 | Cervix | 7 | 53 | 51 | | 51 | 37-55 | I-IV | + | + | + | + | + | + |
| 10 | Colon | 6 | 1586 | 1586 | 823 | 763 | 16-90 | I-IV | + | + | + | + | + | + |
| 11 | Esophagus | 7 | 63 | 62 | 41 | 21 | 24-65 | I-IV | + | + | + | + | + | + |
| 12 | Fibrous | 7 | 25 | 25 | 12 | 13 | 35-67 | I-IV | + | + | + | + | + | + |
| 13 | Gallbladder | 4 | 26 | 26 | 20 | 6 | 37-62 | I-IV | + | + | + | + | + | + |
| 14 | Head/neck | 5 | 12 | 12 | 4 | 8 | 41-66 | I-IV | + | + | + | + | + | + |
| 15 | Intestine | 3 | 15 | 15 | 10 | 5 | 36-68 | I-IV | + | + | + | + | + | + |
| 16 | Kidney | 11 | 123 | 120 | 81 | 39 | 33-65 | I-IV | + | + | + | + | + | + |
| 17 | Liver | | 362 | 358 | 231 | 127 | 36-76 | I-IV | + | + | + | + | + | + |
| 18 | Lung | 30 | 683 | 676 | 437 | 239 | 27-78 | I-IV | + | + | + | + | + | + |
| 19 | Lymph | 13 | 56 | 56 | 31 | 25 | 21-73 | I-IV | + | + | + | + | + | |
| 20 | Oral cavity | 6 | 17 | 17 | 10 | 7 | 28-69 | I-IV | + | + | + | + | + | + |
| 21 | Ovary | 6 | 83 | 83 | | 83 | 32-61 | I-IV | + | + | + | + | + | + |
| 22 | Pancreas | 16 | 222 | 218 | 162 | 56 | 28-74 | I-IV | + | + | + | + | + | + |
| 23 | Penis | 3 | 3 | 3 | 3 | | 45-62 | I-IV | + | + | + | + | + | + |
| 24 | Prostate | 7 | 1606 | 1528 | 1528 | | 35-77 | | + | + | + | + | + | + |
| 25 | Rectum | 7 | 5 | 5 | 4 | 1 | 32-57 | I-IV | + | + | + | + | + | + |
| 26 | Skin | 10 | 122 | 122 | 35 | 87 | 21-79 | I-IV | + | + | + | + | + | + |
| 27 | Spleen | 3 | 6 | 6 | 1 | 5 | 36-48 | I-IV | + | + | + | + | + | |
| 28 | Smooth | 3 | 20 | 20 | 10 | 10 | 35-55 | I-IV | + | + | + | + | + | + |
| | muscle | | | | | | | | | | | | | |
| 29 | (other) Soft | 12 | 120 | 106 | 56 | 50 | 36-66 | I-IV | + | + | + | + | + | + |
| | tissues | | | | | | | | | | | | | |
| 30 | Stomach | 10 | 245 | 245 | 123 | 122 | 36-72 | I-IV | + | + | + | + | + | + |
| 31 | Testis | 5 | 24 | 24 | 24 | | 41-67 | I-IV | + | + | + | + | + | + |
| 32 | Thymus | 8 | 5 | 5 | 4 | 1 | 37-56 | I-IV | + | + | + | + | + | |
| 33 | Thyroid | 3 | 17 | 17 | 10 | 7 | 29-61 | I-IV | + | + | + | + | + | + |
| 34 | Uterus | 1 | 26 | 26 | | 26 | 35-56 | I-IV | + | + | + | + | + | + |
| 35 | Vulva | 3 | 13 | 13 | | 13 | 32-55 | I-IV | + | + | + | + | + | + |
| | Total | 293 | 9972 | 9784 | 3937 | 584 | | | | | | | | |

### Example XIII

### Mechanics of Cytocapsula Growth, Generation and Elongation, and Cytocapsular Oncocell Migration in CCTs

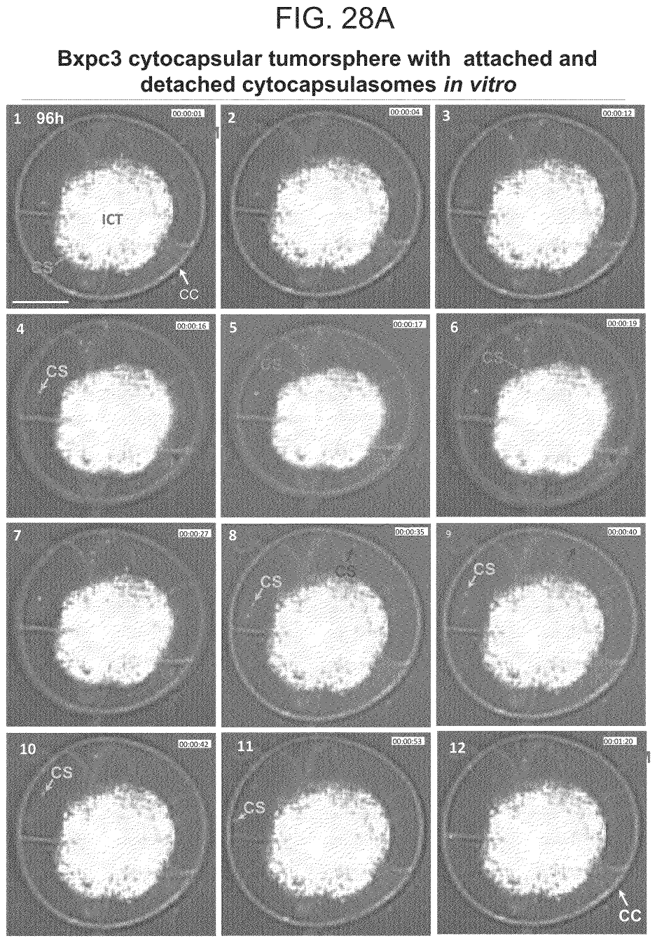

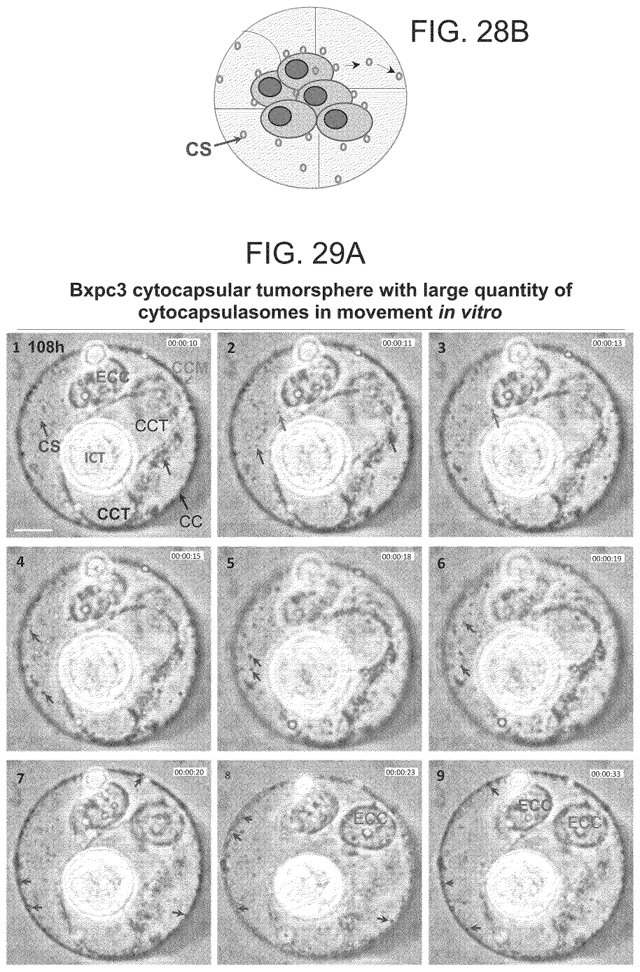

Next, we investigated the mechanics underlying the growth of cultured cytocapsulas around oncocells in vitro. Using inverted bright field microscope, we recorded videos of Bxpc3 tumorspheres in big CCs with wide gaps in the lumens between tumorsphere edge and CC membranes. There are multiple spike-like structures (0.2~0.9 μm in diameter/width, 4~9 μm in length) in the CC lumen (named cytocapsular spikes, CS, FIG. 28A and FIG. 28B). The cytocapsular spikes (checked/examined CCs, n=26; average: n=4~10 CSs per cytocapsular tumor) interconnect the tumorsphere surfaces with the inside face of CC membranes. They point in multiple 3D directions, support the enlarged CC membranes, and maintain CC spherical/irregular morphologies possibly to prevent collapse (FIG. 28A and FIG. 28B).

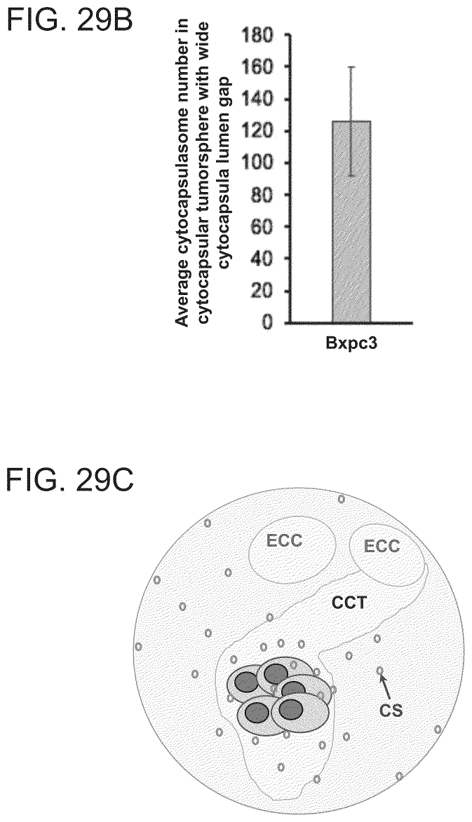

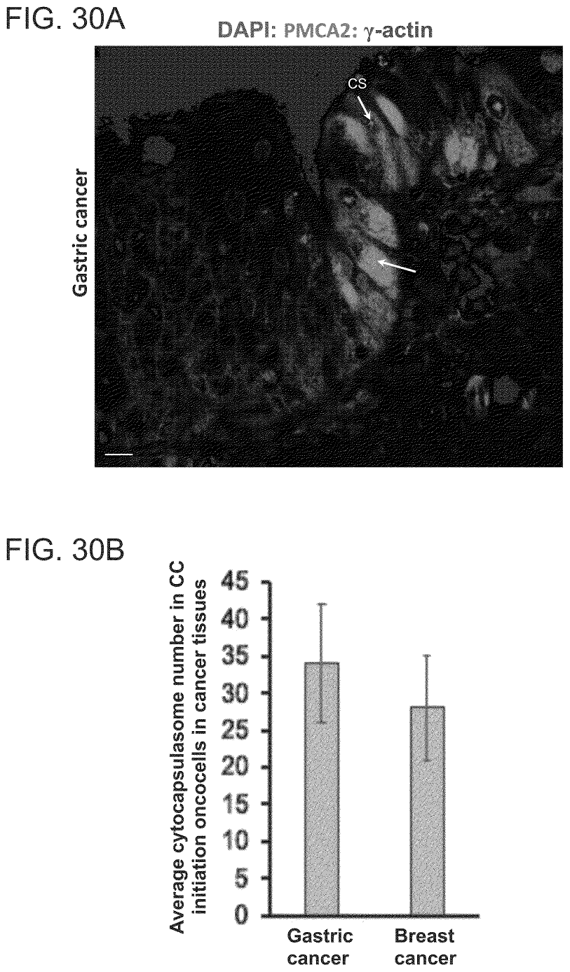

Furthermore, we observed numerous tiny (0.1~0.5 μm in diameter/width), spherical, membrane-enclosed particles (subsequently identified as vesicles containing PMCA2) that attach to and cover the tumorsphere surfaces, indicating that tumorsphere oncocells generate large quantities of this kind of vesicles (n=12 cytocapsular tumorspheres, FIG. 28A and FIG. 28B). These vesicles detach from oncocell surfaces and freely and randomly move in the cytocapsular lumen fluids (FIGS. 28A-28B, FIGS. 29A-29C). Eventually, these vesicles contact, fuse, and integrate into CC membranes, increase CC membrane areas and sizes, and promote CC growth (FIGS. 28A-28B, FIGS. 29A-29C).

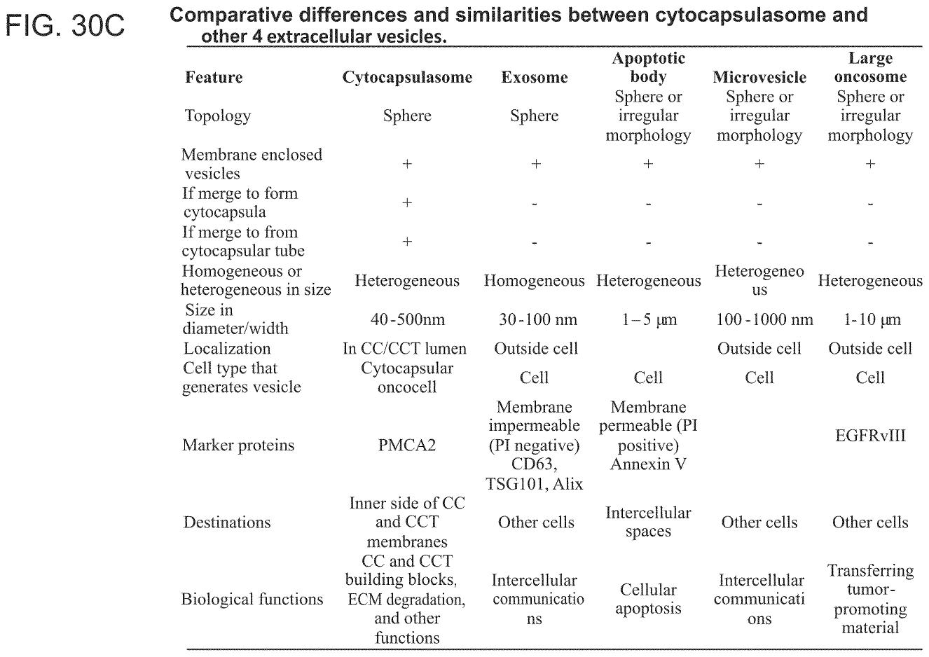

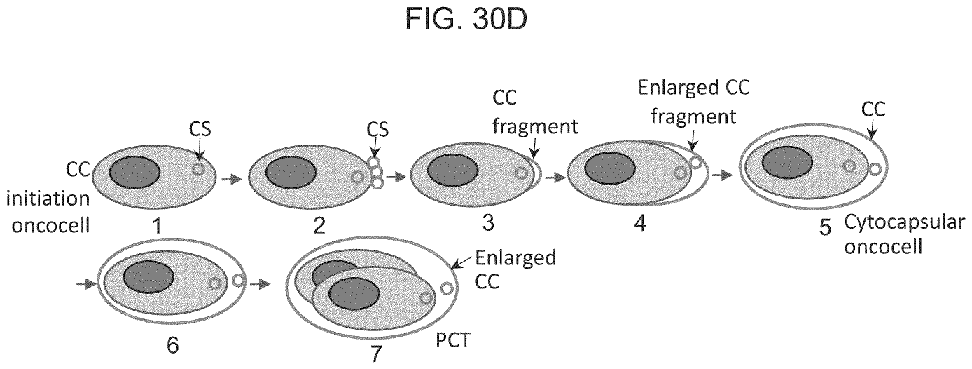

Consistently in vivo, there are many tiny, membrane-enclosed vesicles with high PMCA2 abundance located inside and outside of the cancer cell cytoplasm in both early-stage stomach cancer tissues (FIG. 30A). Subsequently, these vesicles fuse together and generate cytocapsulas enclosing oncocells inside and engender cytocapsular oncocells (FIG. 30A). These vesicles supporting CC generation/growth are present in vitro and in vivo (FIGS. 28-30). We named this kind of tiny and membrane-enclosed vesicles with high PMCA2 abundance, which act as cytocapsular membrane building material delivery cargoes and support CC generation and growth, as “cytocapsulasomes” (FIGS. 30A-30D).

Cytocapsulasomes are initially engendered in the cytoplasm of acytocapsular transformed cells (n=56 samples, FIG. 30A). Subsequently, cytocapsulasomes are released outside the cytoplasm membranes (n=87). These outside cytocapsulasomes fuse together and form small cytocapsular membrane fragments, which subsequently grow into cytocapsulas wrapping the whole oncocells and generate cytocapsular oncocells (n=62 cancer samples, FIG. 30A). The average numbers of detectable cytocapsulasome (CS) per cytocapsular oncocell in stomach and breast cancers are 34±8 CS/cell (n=65) and 28±7 CS/cell (n=120) in vivo, respectively (FIG. 30B). The above observations suggest that acytocapsular transformed cells initially generate cytocapsulasomes in the cytoplasm. Subsequently, cytocapsulasomes are released outside of cellular membrane, randomly diffuse in the lumen and attach to the CC membrane. Then, cytocapsulasomes gradually merge together to form cytocapsulas, and engender cytocapsular oncocells. Cytocapsulasomes function as cytocapsular membrane material cargo delivery carriers to drive cytocapsula growth (FIG. 30D).

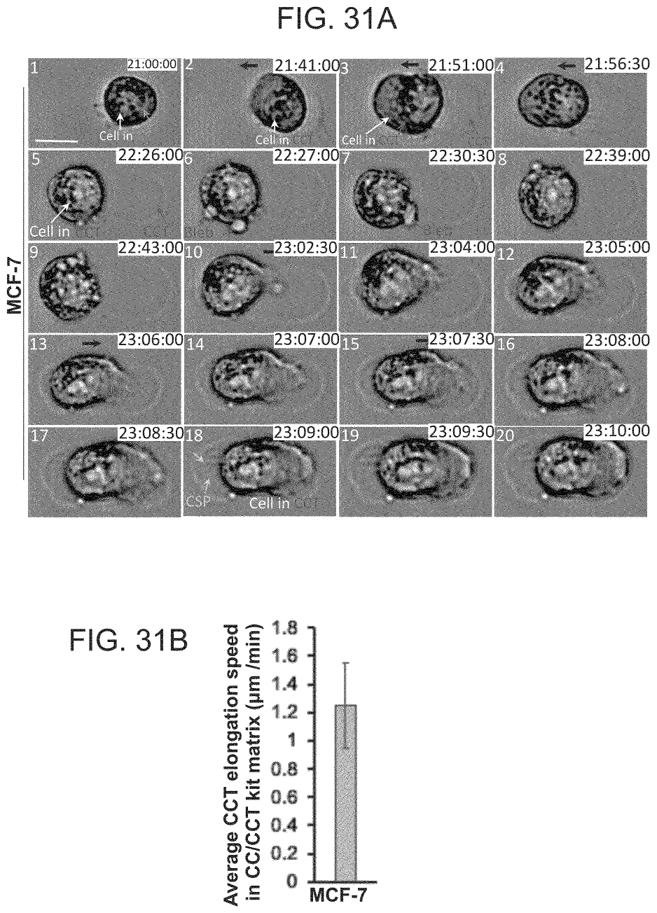

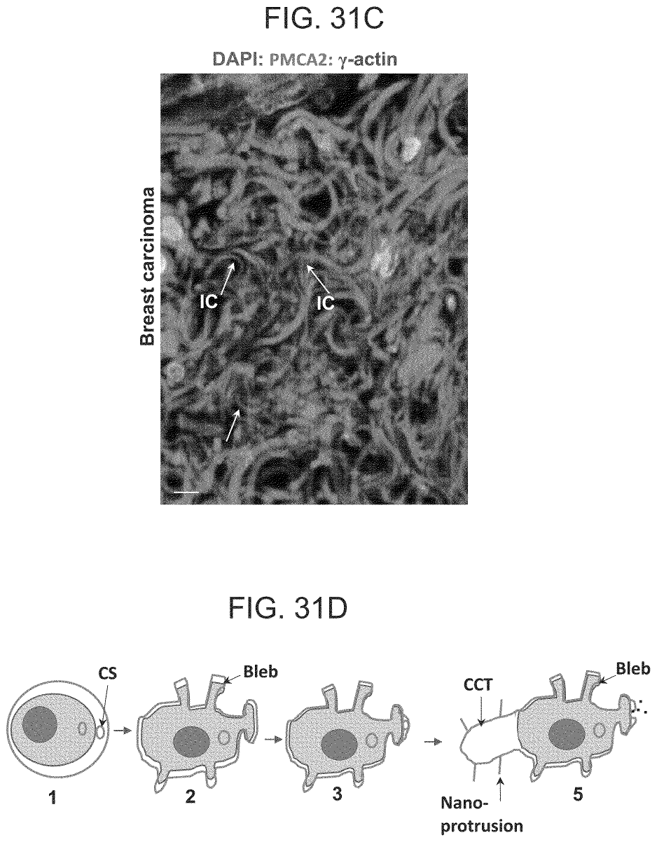

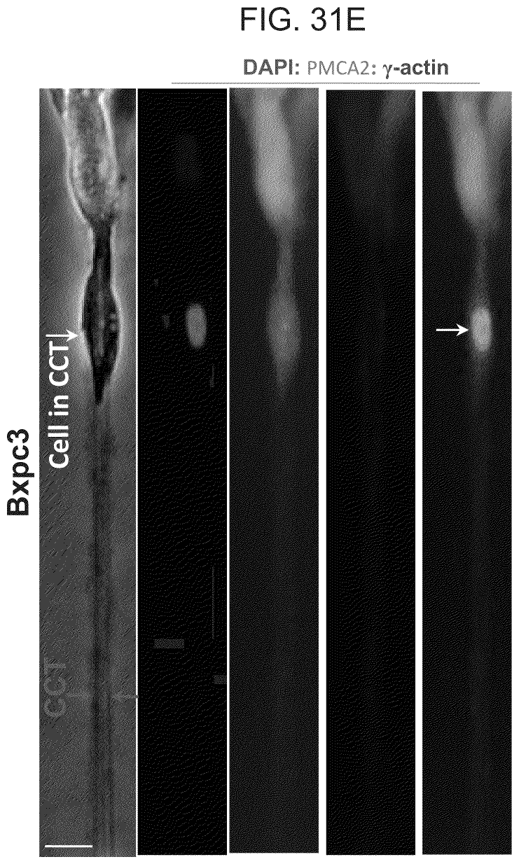

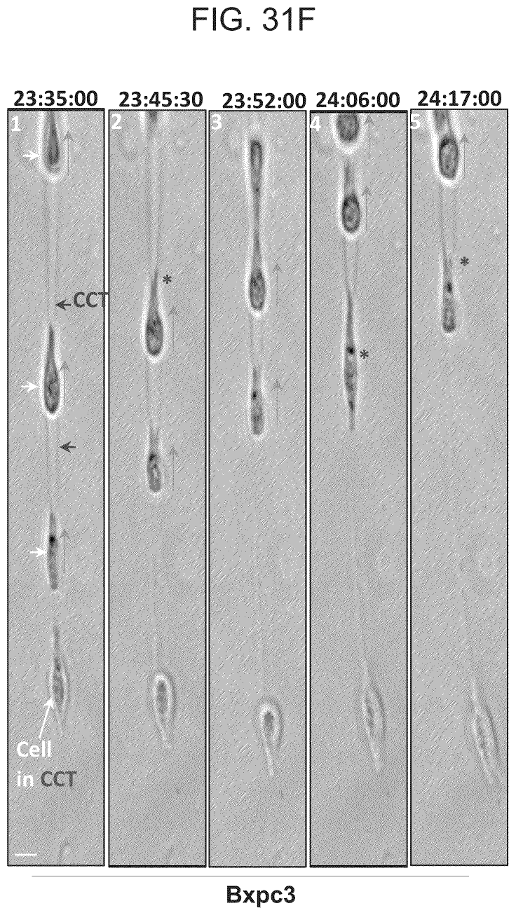

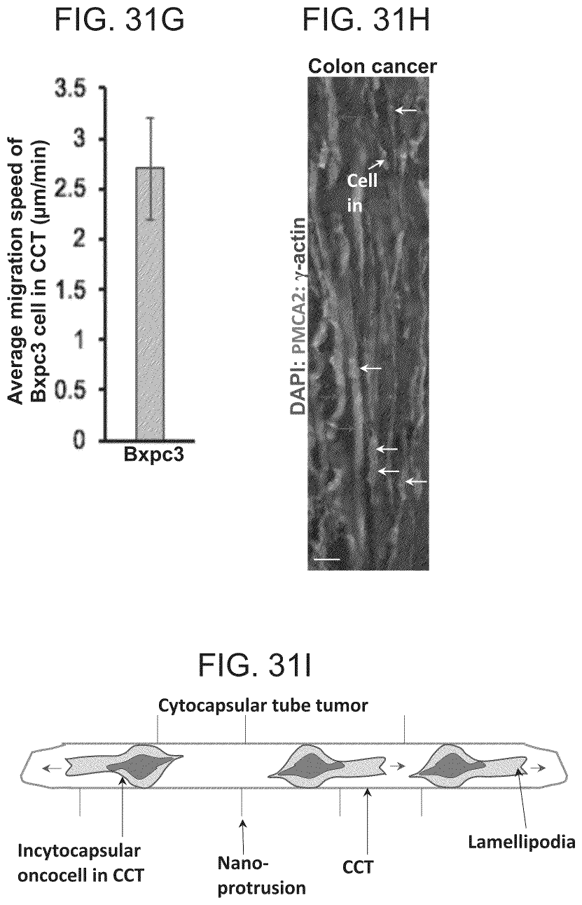

Next, we assessed the mechanics underlying cytocapsular oncocell CCT generation and elongation. Using time-lapse DIC microscopy, we investigated cytocapsular oncocell CCT generation and elongation in vitro. Initially, single MCF-7 breast oncocells generate CCs, the membrane of which tightly wraps around the oncocell. With bleb-based motilities, single cytocapsula oncocells in CCs push CC membranes forward, deform CC membrane shapes into tube-like morphologies, and generate short CCT fragments (FIG. 31A, panels 1-3). The initial CCT fragments are well anchored in the viscous CC/CCT culture kit matrix and maintain the wide tube shapes without being stretched and contracted into thin and long CCT tail shapes (FIG. 31A, panels 2-3). The cytocapsular oncocells constantly generate cytocapsulasomes, which merge and integrate into the front part of the CC membranes. They tightly keep contact with oncocell membranes, and increase CC membrane areas. Cytocapsular oncocells constantly and dynamically generate many transient blebs in various sizes. Blebs repeatedly protrude and retract in the CC, sense the extracytocapsular environments in many 3D directions, and choose or decide the motility directions. After cytocapsula creation, oncocells migrate backward in the CCTs they generated. They engender several short cytocapsular spikes in the CCT lumens, which link the rear of the oncocell and CCT membranes (FIG. 31A). Subsequently, when the single cytocapsular oncocells migrated backward in the established CCTs, it switched into a lamellipodia-based motility format (FIG. 31A, panels 11-20). Average CCT elongation speed of MCF-7 cytocapsular breast oncocells in CC/CCT culture kit matrix is 1.25±0.3 μm/min (n=3 cells, FIG. 31B).

Next, we investigated cytocapsular oncocell CCT generation and elongation in vivo. In the early stage of breast cancer (FIG. 31C) there are many initial cytocapsular oncocell CCTs that have long and thin CCT tails (0.1~1 μm in width, IC). In the initiation of CCT regeneration in AMCC phase (FIG. 22E), a single breast acytocapsular oncocell engenders multiple CCTs with thin-tails (0.2~2 μm in width) where it tries multiple different migration directions during cancer metastasis. This indicates that the initial CCT fragments are stretched and contracted by the single cytocapsular breast oncocells when they move forward while the initial CCT fragments are not well anchored into the ECM (FIG. 22E and FIG. 31C). Subsequently, when the CCTs are well anchored in the ECM by extended CCT nano-protrusions, the CCTs remain consistently maintained at 3~6 μm in diameter/width in solid cancers (FIG. 31C). The above in vitro and in vivo observations suggest that single cytocapsular oncocells in CCs employ a bled-based sensing and motility format. This implies a possible mechanism of how cytocapsulasomes effect directions and promote promote forward movement, engender cytocapsulasomes to support CC membrane size increase, push and deform CC membranes into tube-shaped morphologies, and generate and elongate CCTs (FIG. 31D). The initial CCT fragments frequently have long and thin CCT tails in vivo.

Next, we investigated mechanics of cytocapsular oncocell migration in CCTs. Single Bxpc3 pancreas cytocapsular oncocells migrating in long CCTs (3~6 μm in diameter), appear squeezed by the stretched and contracted CCT membranes and molded into long, thin, spindle-shaped morphologies. Single cytocapsular oncocells appear usually in single cell mesenchymal migration format in CCTs (FIG. 31E). Furthermore, using time-lapse DIC technologies, we investigated the dynamic cell migration activities in CCTs with multiple MCF-7 breast cytocapsular oncocells migrating in a long CCT. In most instances, multiple cytocapsular oncocells in CCTs are in a single epithelial migration format, not in collective migration mode. The polarized, thin and long cytocapsular oncocells in CCTs migrate forward with periodic protrusion and retraction of the leading lamellipodia and movement of the blunt cell rear. Sometimes, the long lamellipodia are completely retracted and the cells transiently appear in spherical morphologies in CCTs. CCT membranes always tightly wrap and adhere to cancer cell cytoplasm membranes and dynamically increase/decrease the CCT diameter/width locally, displaying considerable CCT membrane elasticity (FIG. 31F). The polarized cytocapsular oncocells in CCTs can freely switch the migration direction back and forth and can thus migrate bi-directionally in CCTs. Here, multiple cytocapsular oncocells freely and bi-directionally migrate in a long CCT, with dynamic cellular morphologies. The elastic CCT membranes shelter from obstacles in the heterogeneous extracytocapsular matrix outside, and provide membrane-enclosed and protected freeways for cytocapsular oncocell migration inside (FIGS. 31E and 31F). The average cell migration speed of multiple single Bxpc3 pancreas cytocapsular oncocells in CCT in vitro is 2.7±0.5 μm/min (n=10 cells (FIG. 31G). Consistently, in the long (straight or curved) cytocapsular colon oncocell CCTs (3~6 μm in diameter) in colon carcinoma tissues, cytocapsular colon cancer cells migrating in CCTs, usually adapt single, long, thin and spindle-shaped morphologies in vivo (CCT, n=256; tissues, n=34; FIG. 31H). This indicates that cytocapsular oncocells in migration in CCTs in vivo use a single mesenchymal migration format (FIG. 31H). The above observations suggest that cytocapsular oncocells can freely, dynamically, and bi-directionally migrate in membrane-enclosed and protected CCTs in a single mesenchymal migration format and in thin and long morphologies in vitro and in vivo. A CCT with multiple or numerous oncocells inside is topologically and bio-functionally a long and tube-shaped cytocapsular tumor, and is thus termed a “cytocapsular tube tumor” (FIGS. 31H-31I).

### Example XIV

### Primary Cytocapsular Tumor Network System Progression

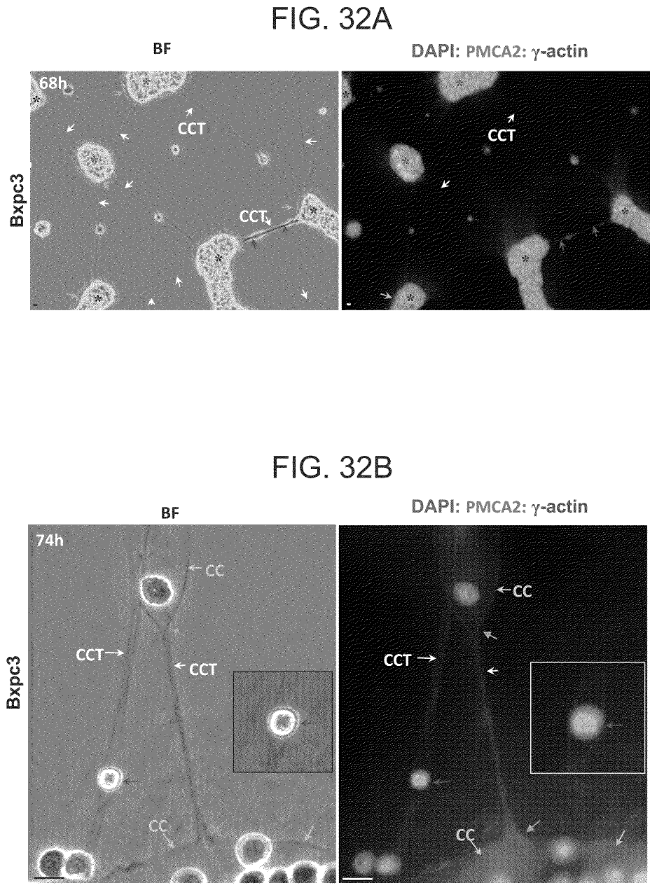

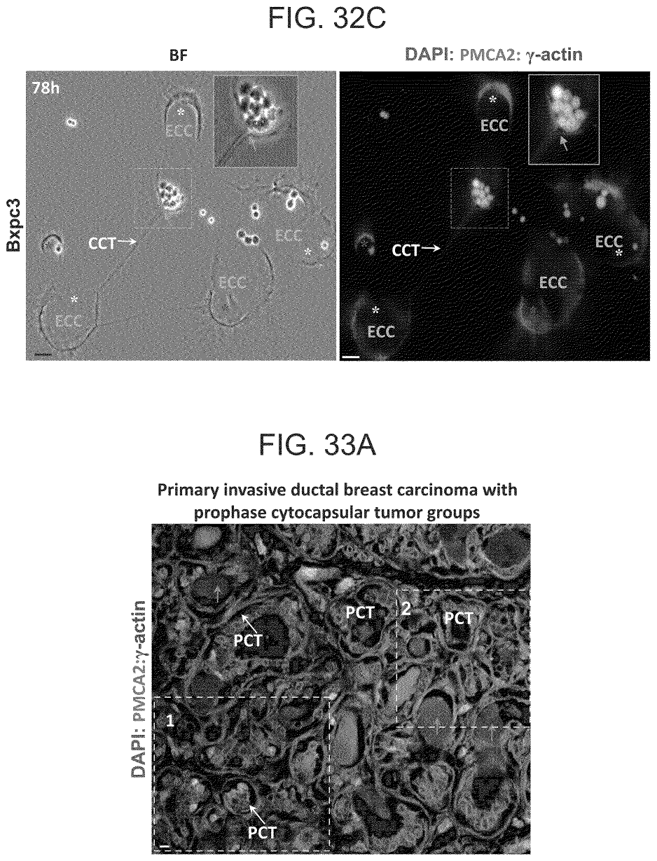

Primary malignant tumors are origins of metastatic (secondary) cancers. Thus, we asked how primary cytocapsular tumors progress in primary niches. We assessed cytocapsular tumorsphere metastasis and secondary cytocapsular tumorsphere growth in vitro. In the CC/CCT culture kit matrix, at 68 h, single Bxpc3 cytocapsular oncocells proliferate in CCs and grow into big primary cytocapsular tumorspheres (CTs, FIG. 32A). Two or more primary CTs merge together via CC membrane contact, contact-interface degradation, and open connection formation, and form long and irregular-shaped CTs enclosed in long and big CCs (FIG. 32A). Cytocapsular oncocells in enlarged CCs push CC membranes, elongate and generate CCTs. CCTs interconnect and form CCT networks (FIG. 32A). The CCT networks interconnect all primary and secondary CTs and form a cytocapsular tumorsphere network systems (CTNS) in a well of 6-well plate (FIG. 32A). Acytocapsular oncocells can enter CCTs via alloentry. Incytocapsular oncocells perform spontaneous ecellulation and become acytocapsular oncocells beyond CCs/CCTs (FIG. 32A). The ecellulated acytocapsular oncocells can regenerate new cytocapsulas and CCTs (FIG. 32B). Incytocapsular oncocells metastasize via CCT networks (FIG. 32B). In addition, cytocapsular oncocells metastasize and reside in CCT interconnection nodes, proliferate and grow into secondary cytocapsular tumorspheres, which are integrated into the established CTNSs (n=38 checked secondary cytocapsular tumorspheres (FIGS. 32A-32B). Incytocapsular oncocells migrate and translocate in CTNSs via CCT networks (FIG. 32B). Cytocapsular tumorspheres' spontaneous ecellulation engenders acellular CT cytocapsular parts and acellular CCT fragments, making the open CCT connections between CTs visible (FIG. 32B). At 78 h, all the primary and secondary CTs in a well of 6-well plates are interconnected and covered by the integrated cytocapsular membrane systems, which are composed of cytocapsular tumorsphere CCs and CCT networks (CTNS number, n=55, FIG. 32C). These observations suggested that: (1) primary CT metastasize, generate CCTs, CCT networks and primary CTNSs; (2) metastasis of cytocapsular oncocells develop into secondary cytocapsular tumorspheres, and form secondary CTNSs; (3) ecellulation and alloentry allow oncocells to bidirectionally evict from and enter into CCs/CCTs; and (4) primary and secondary CTNSs integrate via CCT networks and form a dynamic and integrated primary and secondary CTNS enclosed in cytocapsular membrane systems in vitro.

Next, we investigated primary cytocapsular tumor network systems (CTNSs) in vivo, following subsequent development stages as outlined in FIG. 25C.

### Prophase CT (PCT) Formation:

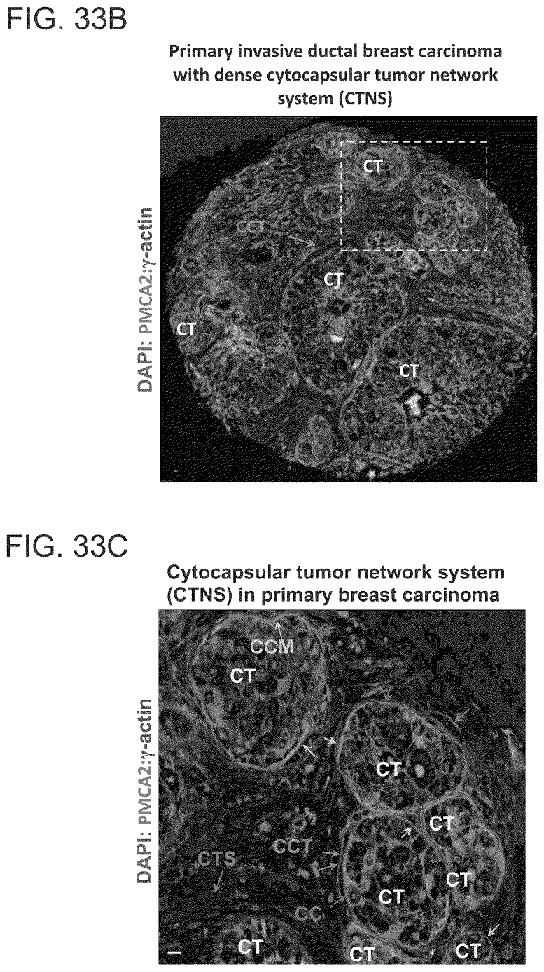

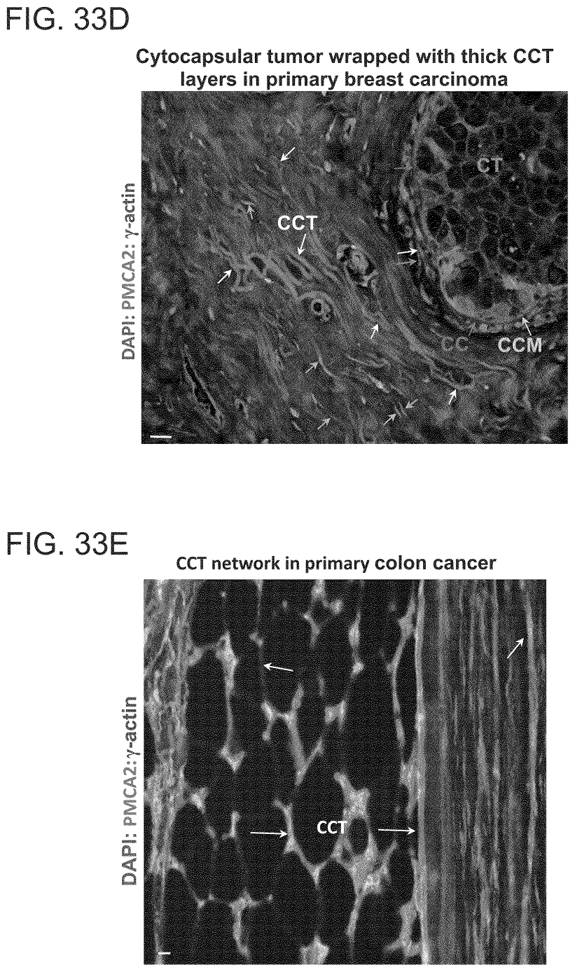

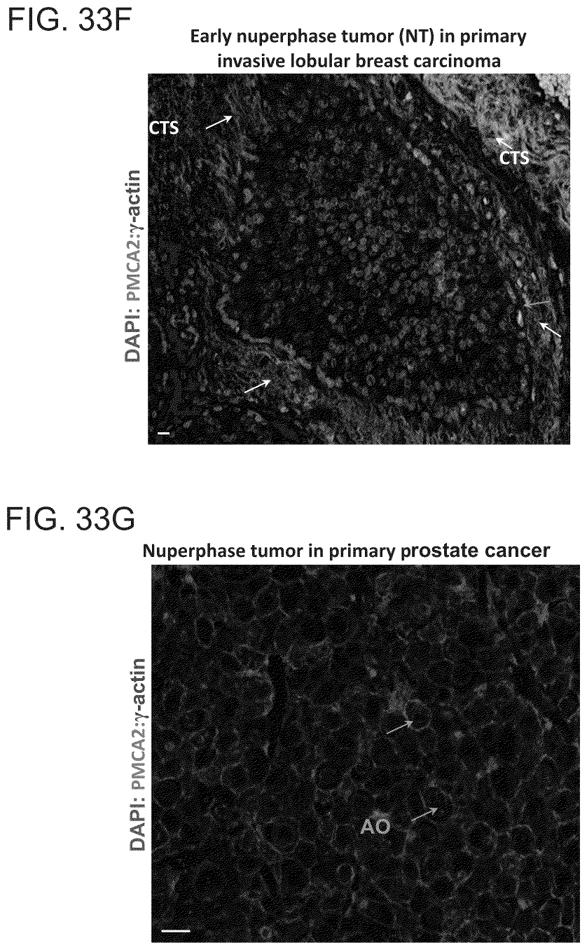

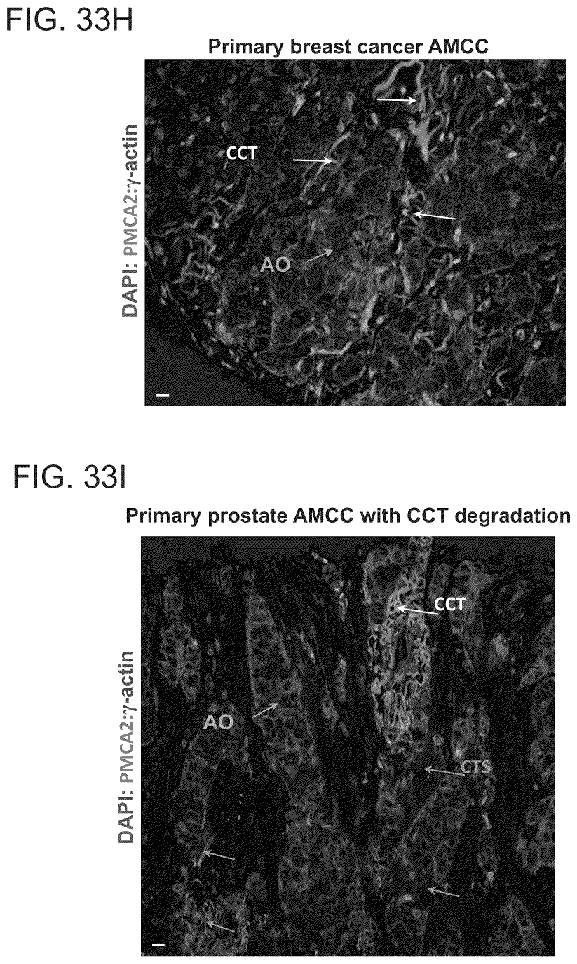

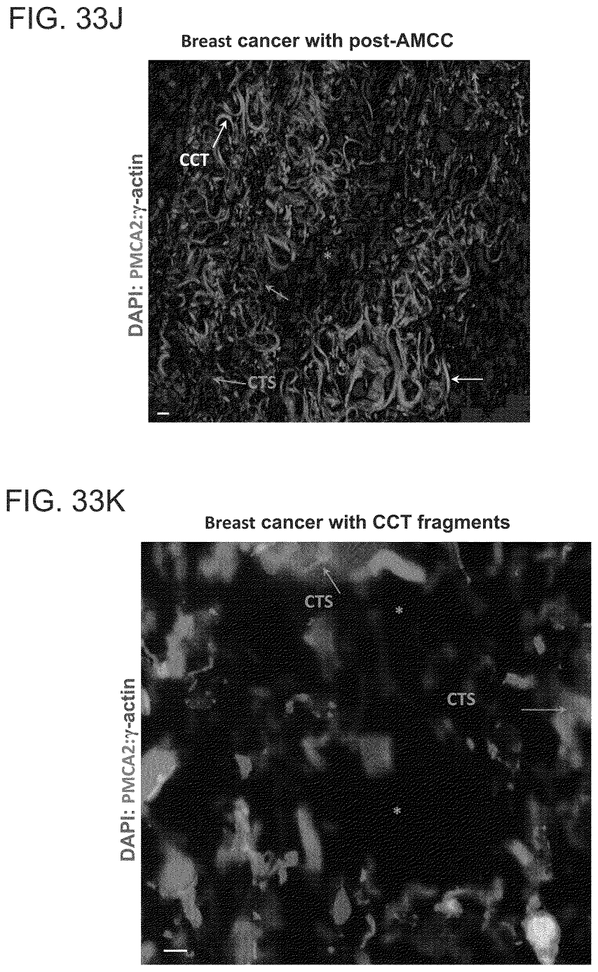

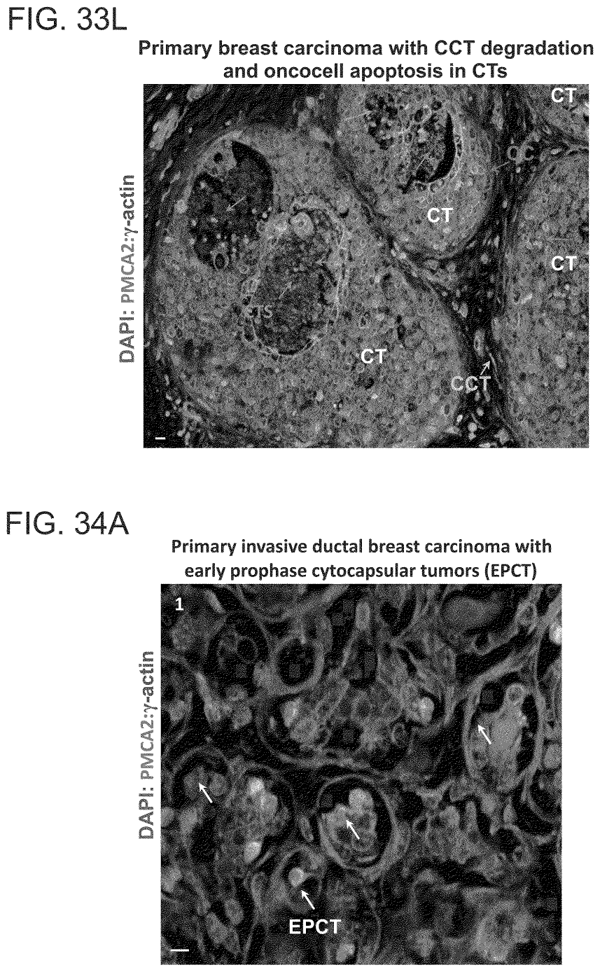

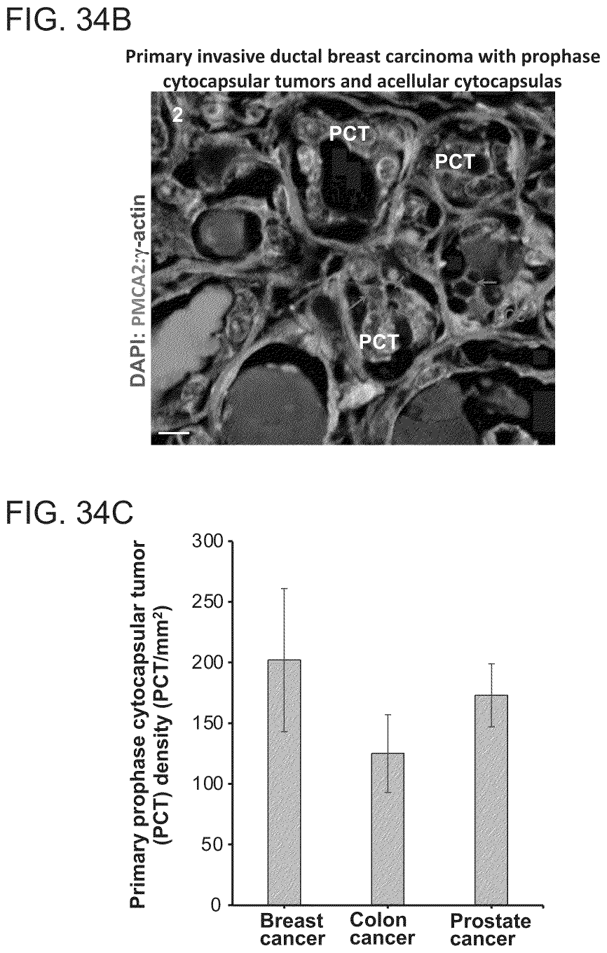

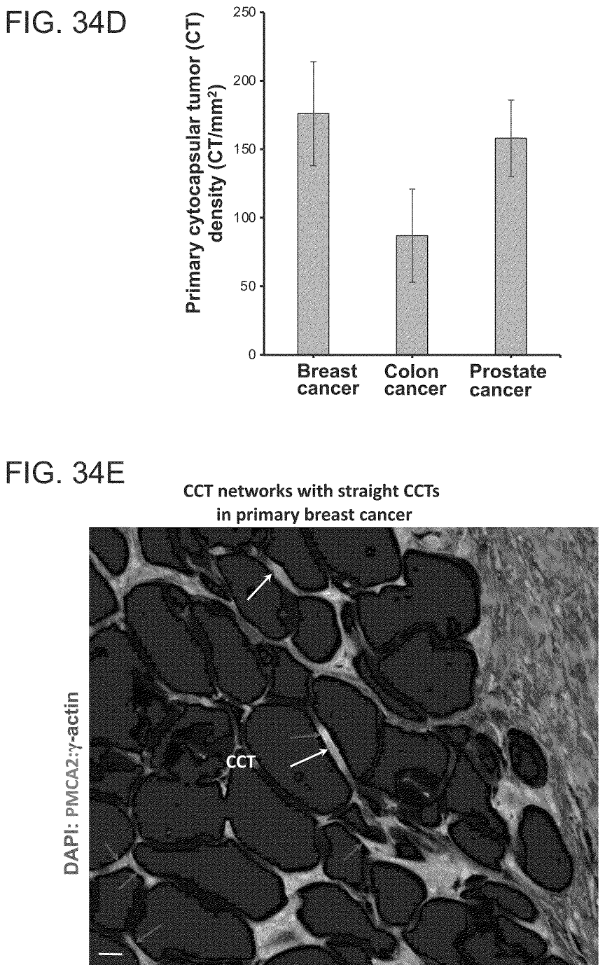

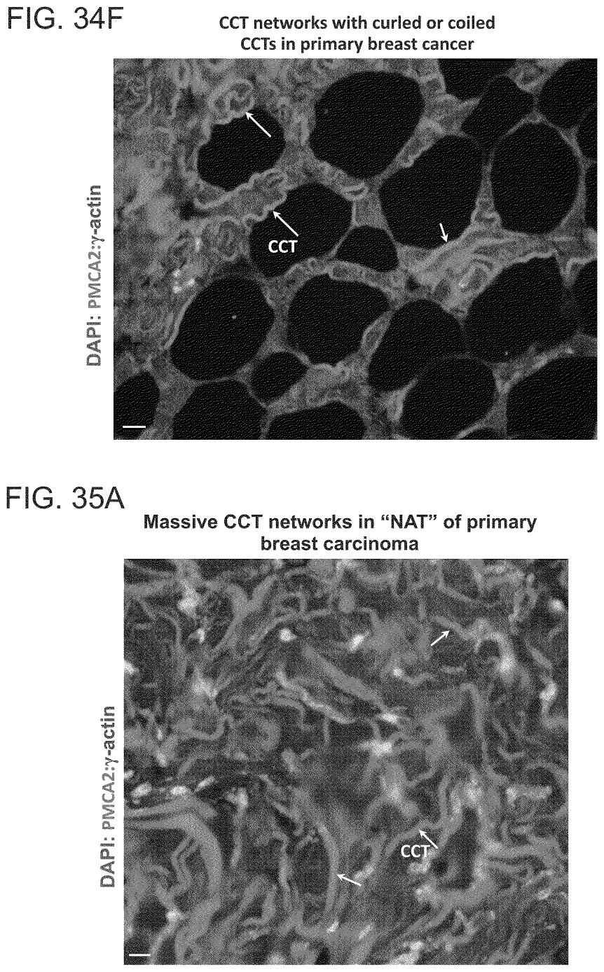

In early-stage primary breast cancers, there are many spherical or irregular-shaped prophase cytocapsular tumors (PCTs) in variable sizes (25~120 μm in diameter/width, FIG. 26F, FIG. 33A and FIGS. 34A-34B). The average prophase cytocapsular tumor (PCT) densities of early-stage cancers in breast, colon, and prostate are 202±59 PCTs/mm², 125±32 PCTs/mm², and 173±26 PCTs/mm², respectively (specimens, n=3~6, 1 specimen/patient, 1-2 subtypes/cancer type, FIG. 34C).

### PCT to CT:

Subsequently, the PCTs develop into spherical or irregular-shaped cytocapsular tumors (CTs) in variable sizes (50~320 μm in diameter/width, FIGS. 33B-33C). The average CT densities of early-stage cancers in breast, colon, and prostate are 176±38 CTs/mm², 87±34 CTs/mm², and 158±28 CTs/mm², respectively (specimens, n=5~12, 1 specimen/patient, 3-4 subtypes/cancer type) (FIG. 34D). These data indicate that PCT and CT densities in the early primary solid cancers are statistically high. Consistent with cytocapsular tumorsphere merge in vitro, two or more small PCTs/CTs in vivo can merge into middle or big-sized PCTs/CTs (FIG. 26F and FIG. 33A). The CTs with >50 μm in diameter/width start to generate a few cytocapsular tubes (CCTs) outside. Cytocapsula tumors with >70 μm in diameter/width engender a lot of CCTs outside and form thick surrounding CCT layers wrapping cytocapsula tumors (FIG. 26G and FIG. 33D). The dense CCT layers present various thicknesses (30~803 μm in thickness) (FIG. 33D). Straight, curved or coiled CCTs intensively interconnect and form 3D CCT networks, which broadly interconnect primary CTs in the primary cancer niches (FIG. 33E and FIGS. 34E-34F). These observations suggest that cytocapsular tumors in the primary niches are physically interconnected via 3D CCT networks and form primary cytocapsular tumor network systems (CTNSs) in vivo.

### CC Degradation:

Subsequently, CCs and CCTs of cytocapsular tumors in size of >800 μm in diameter/width degrade into strands followed by disappearance, engendering acytocapsular malignant tumors/oncocell masses with high cell density and without CCs or CCTs (FIG. 33F).

### Nuperphase Tumor:

Uncontrolled proliferation of acytocapsular oncocells generate big and irregular-shaped acytocapsular malignant tumors/oncocell masses (up to >2 cm in width) in the checked sectioned specimens (FIG. 33G).

### AMCC:

The acytocapsular status of these big malignant tumors/oncocell masses is transient. Subsequently, some acytocapsular oncocells regenerate a few or many new straight, curved or coiled CCTs, and form CCT network bunches and masses, which invade into and scatter in the dense acytocapsular oncocell masses, making a complex mixed by dense oncocell masses and CCT networks but without an enlarged CC wrapping them (FIG. 33H). CCT bunches and masses create many CCT mass cavities (cross section: in round, oval or irregular shapes, 30~200 μm in diameter/width; longitudinal section: in straight, curved, or irregular column morphologies, 80~850 μm in length), which are interconnected and form CCT mass cavity compounds (FIG. 23C, panel 3 and FIG. 33H). Acytocapsular oncocells around CCTs perform alloentry, invade into CCTs, and disseminate via CCTs (FIG. 23C, panel 3 and FIG. 33H).

AMCC with CCT Degradation:

Subsequently, CCTs degrade and disappear, leaving CCT mass cavities filled with intercellular fluids (FIG. 33I). Meanwhile, some acytocapsular oncocells regenerate new CCTs (FIG. 33I). CCT degradation and regeneration coexist in the Acytocapsular oncocell Mass-CCT network Complex (AMCC) in vivo (FIG. 33I).

### Post-AMCC:

Subsequently, after many oncocells enter CCTs and depart away via CCTs, there are only a few oncocells left, and many CCTs (FIG. 33J). Then, CCTs degrade, decompose and disappear (FIG. 33K). On the other hand, CTNSs in primary niches at the late cancer stage still remain (FIG. 33L). Some small or middle-sized CTs present oncocell apoptosis and CCT degradation inside (FIG. 33L). The above observations suggest that: 1) primary CTs develop into primary CTNSs in vivo, 2) CC/CCT degradation, acytocapsular oncocell proliferation, and CC/CCT regeneration generate AMCC, 3) primary CTNSs are dynamic systems including CCT alloentry, ecellulation, CC/CCT generation, degradation and regeneration, and dissemination of cytocapsular oncocells via CCTs.

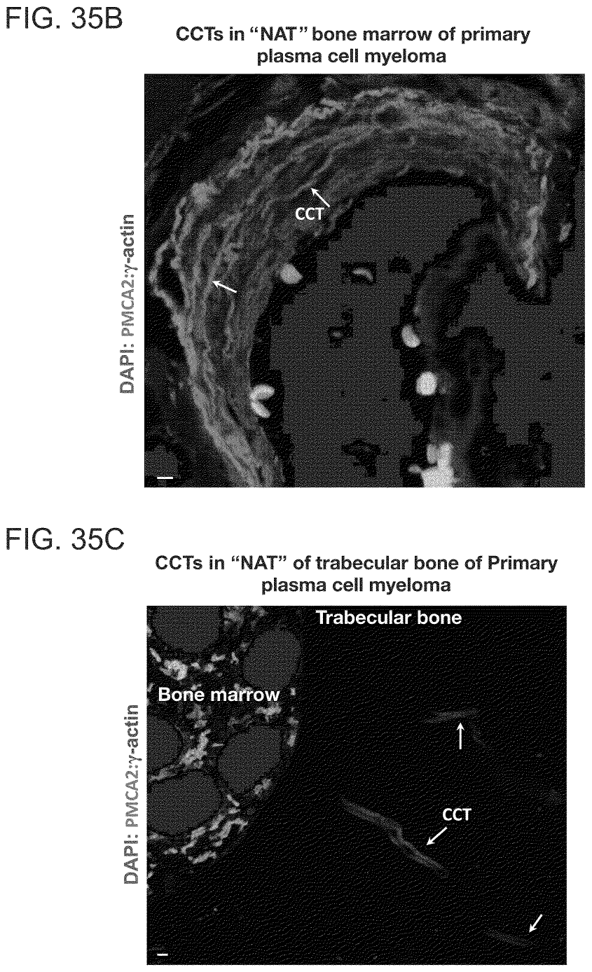

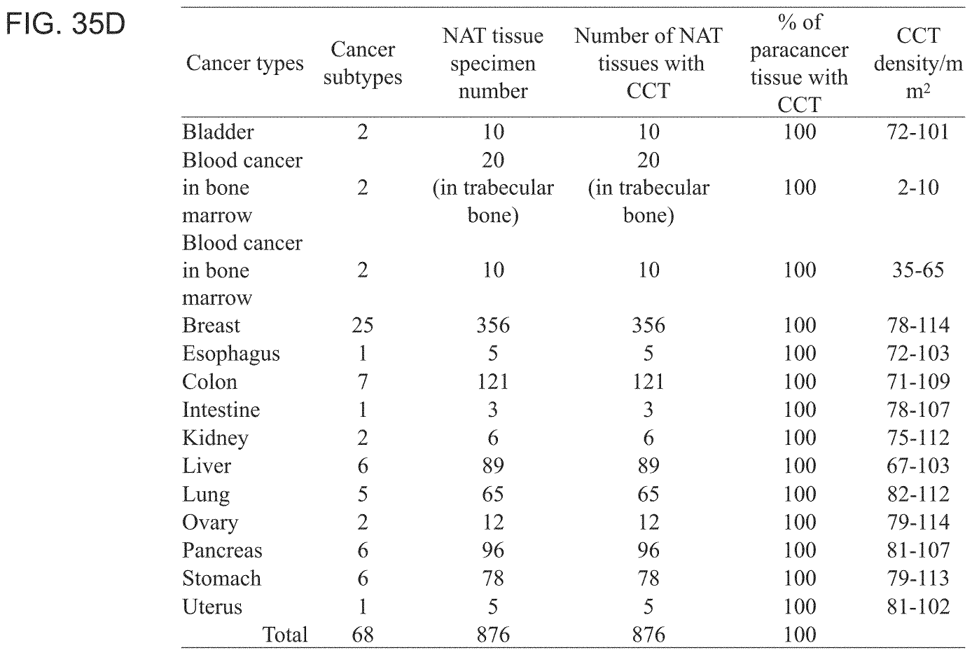

Normal tissues adjacent to the tumor (NAT) are necessary sites that CCTs have to go through if they expand beyond. Indeed, there are large quantities of CCTs that aggressively invade into and go through the NATs in one or multiple 3D directions in breast cancer tissues (FIG. 35A), many CCTs in the bone marrow NAT of plasma cell myeloma (FIG. 35B). Even in the hard tissue NAT of trabecular bone of plasma cell myeloma, there are a few CCTs (FIG. 35C). In the examined NATs of 68 subtypes of cancers, 100% of them harbor a lot of CCTs and networks with cytocapsular oncocells in migration inside (FIG. 35D). CCT density in soft tissue cancers can be up to 114 CCT/mm² and even in hard tissue (bone) cancers can be up to 10 CCT/mm² (FIG. 35D). The above observations (Table 6 and FIGS. 35A-D) suggest that cytocapsular tumor progression in the primary niches includes 7 major successive stages: 1) generation of transformed acytocapsular oncocells, 2) acytocapsular oncocell devolution and generation of cytocapsular oncocells, 3) generation of prophase cytocapsular tumors and cytocapsular tumors, 4) formation of CCT networks and primary CTNSs, 5) cytocapsular oncocell metastasis via CCTs and go through NATs, 6) CC/CCT degradation and AMCC, 7) CCT regeneration, alloentry, ecellulation, and dynamic CTNS formation with continuous cytocapsular tumor metastasis and CTNS regeneration.

### Example XV

### Cytocapsular Oncocell Metastasis in Human Tissues In Vivo

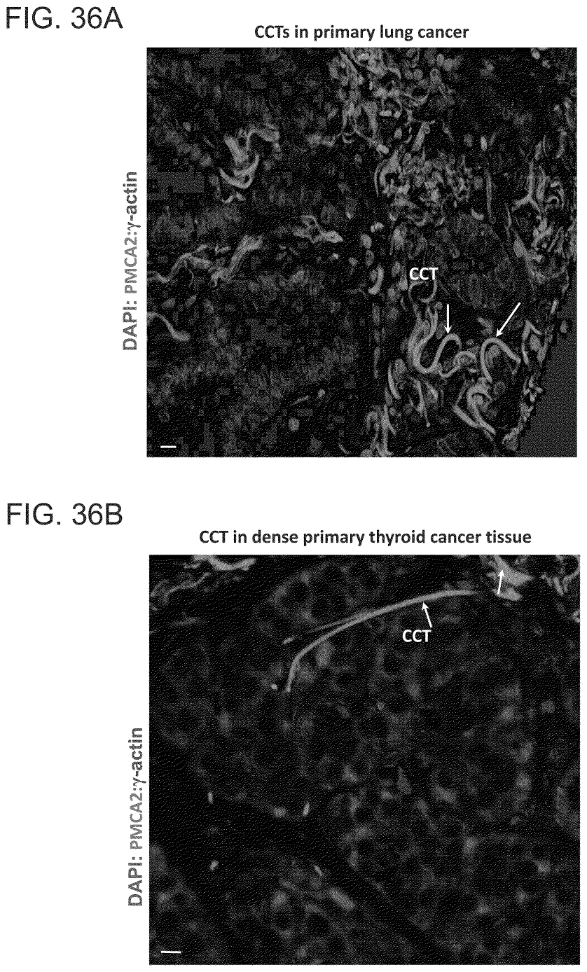

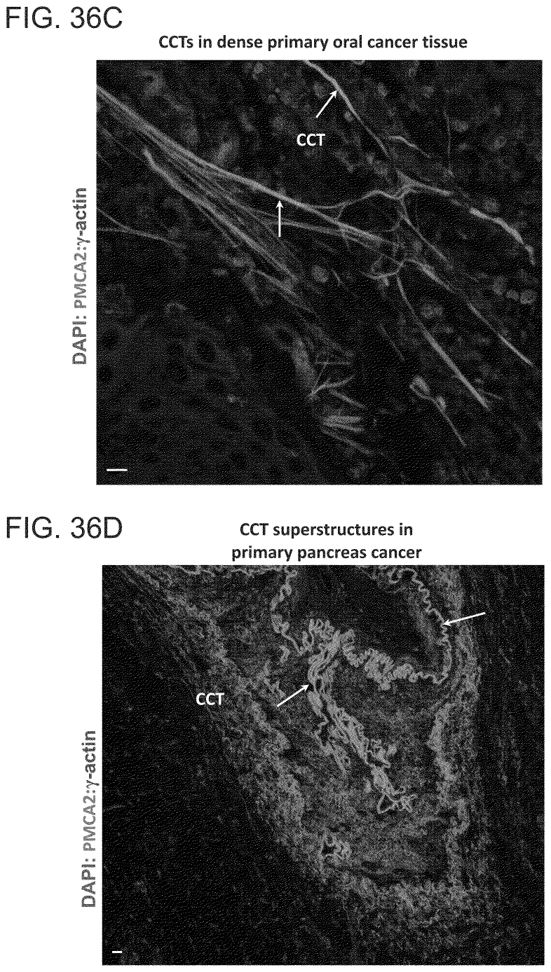

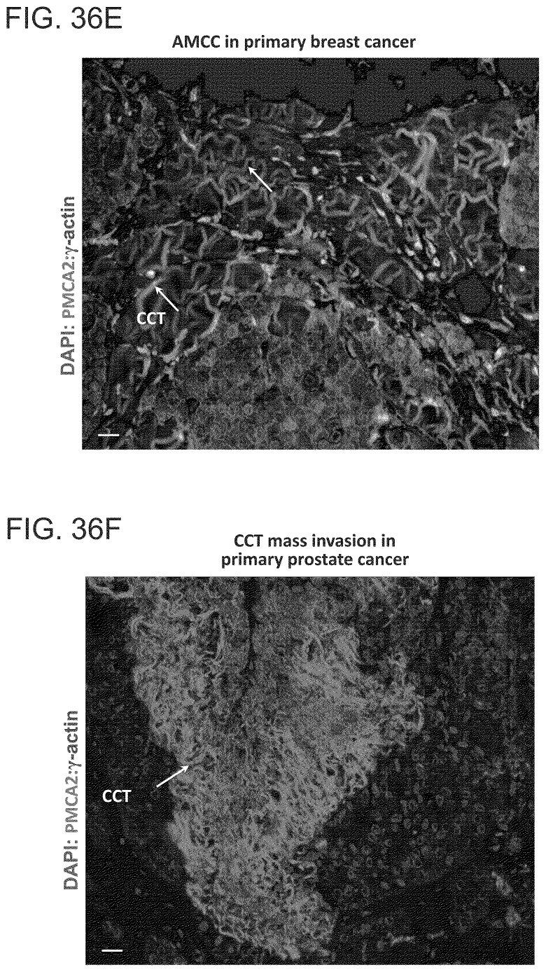

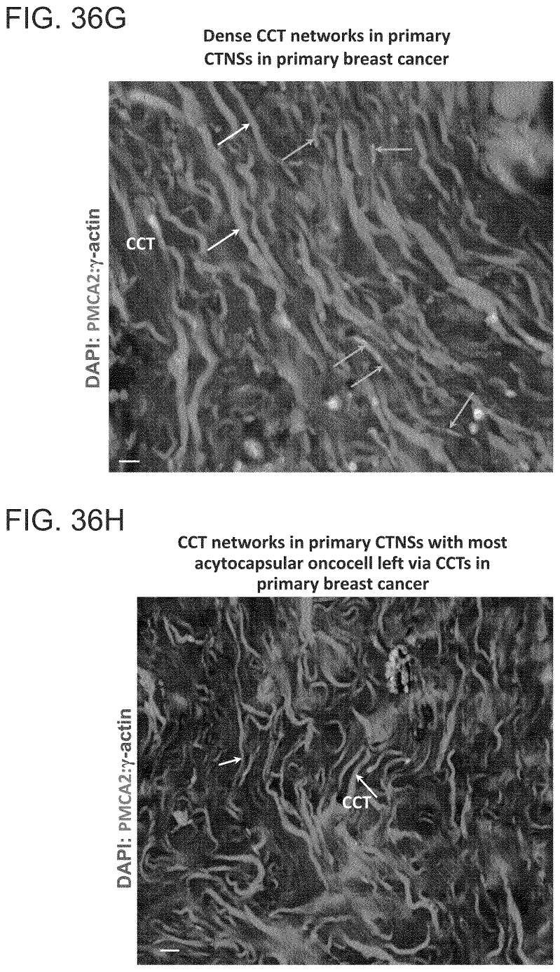

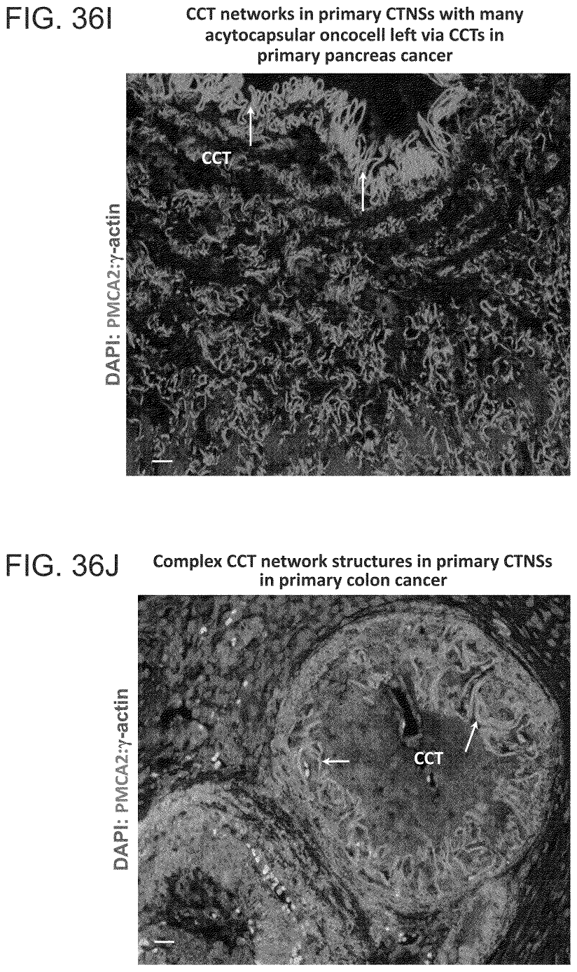

Most cancer death are due to metastases (Gerstberger S, et al., Metastasis. Cell. 186, 564-1579 (2023)). Thus, we assessed how cytocapsular oncocells in CCTs disseminate across diverse tissues and organs in vivo. Single CCTs invade into and spread not only in loose (FIG. 36A) and compact (FIGS. 36B-36C) tissues, but also in hard tissues of trabecular bone (FIGS. 35B-35C), suggesting that cytocapsular oncocell in single CCTs can invade into and go through various kinds of tissues with diverse cell densities and ECM/matrix hardness. Single CCTs can be highly curved and very long (FIG. 36D). 3D CCT networks enhance collective cytocapsular oncocell metastases in and across various kinds of tissues (FIGS. 33A-33L and FIGS. 36E-36F). Massive and compact CCT network bunches increase metastasis freeway density and elevate cytocapsular oncocells dissemination efficiency (FIGS. 36G-36I). Super-large structures of CCT networks facilitate the acytocapsular oncocells in the compact tumors/oncocell masses to enter CCTs and disseminate via CCTs (FIG. 36J).

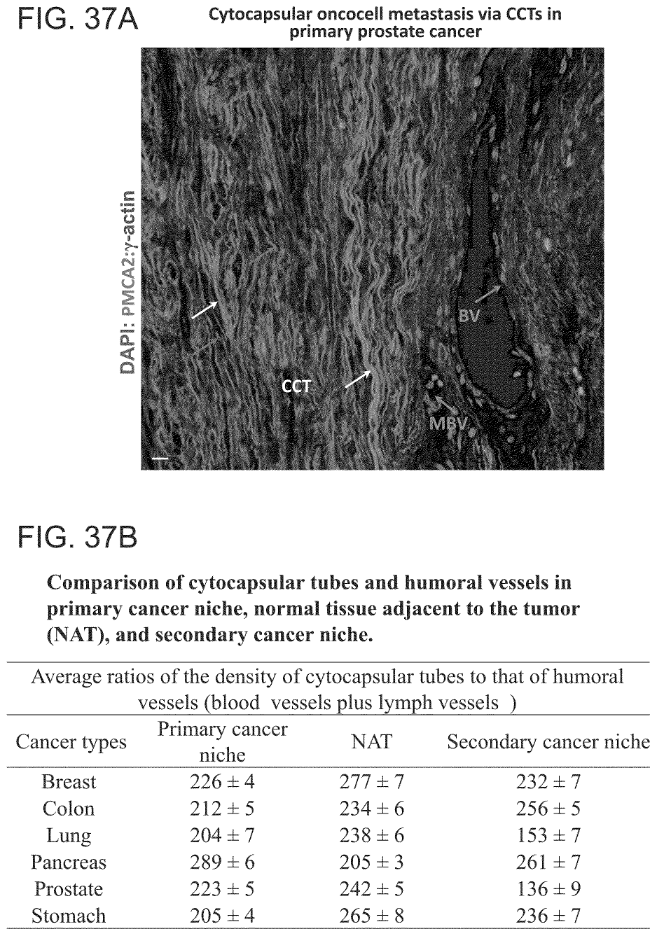

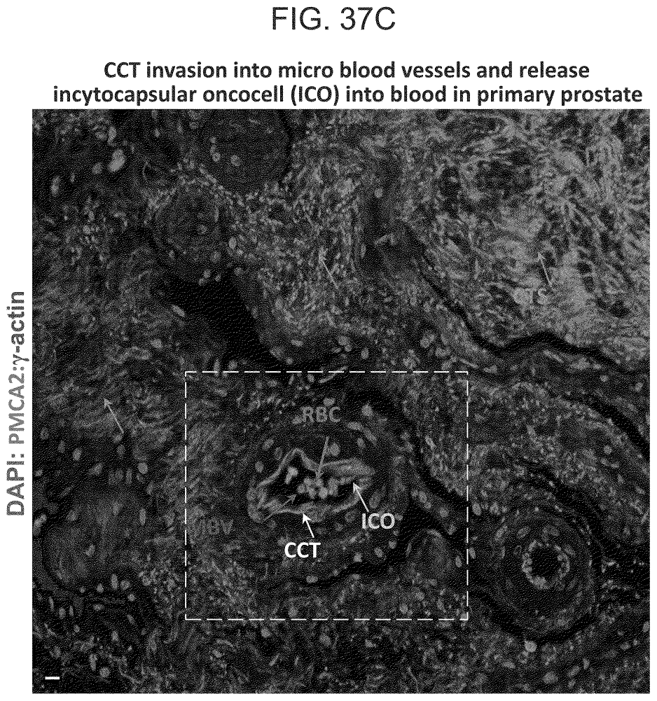

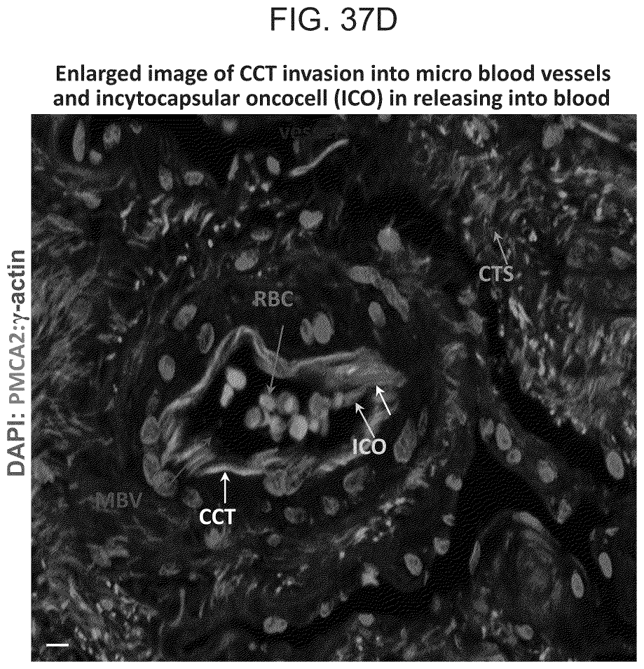

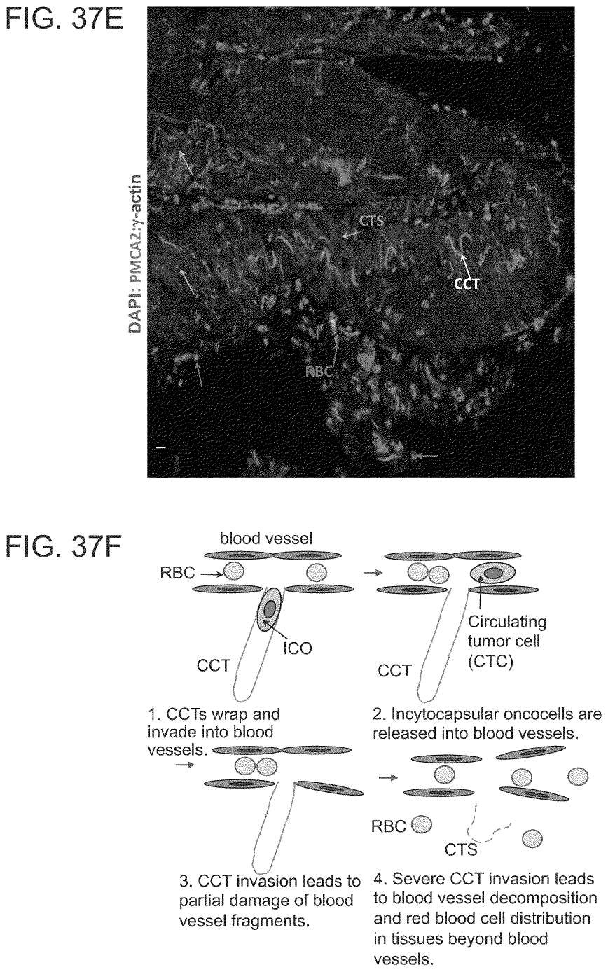

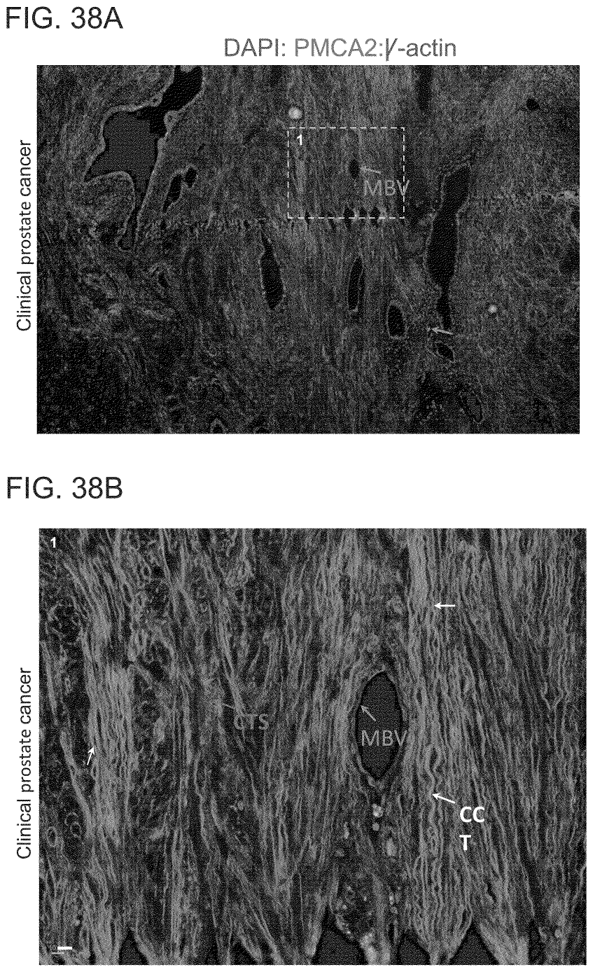

Cytocapsular oncocells and CCT networks observed in bone marrow (FIG. 27B, FIG. 35B and Table 6), lymph nodes (Table 6), spleen, and thymus (Table 6) suggest that CC/CCT membranes can effectively shelter immune cells and their attacks outside, and therefore protect cytocapsular oncocells inside. CCT networks are presented in 290 subtypes of cancers and in 34 kinds of human tissues (FIGS. 27A and 27B, Table 6, FIGS. 35 and 36), suggesting that CCTs effectively cross all kinds of human tissues and organs for cytocapsular tumor metastasis. Large quantities of CCTs are found outside humoral vessels (FIG. 37A and FIGS. 38A-38B). Furthermore, in the primary niches, NATs, and secondary niches, the average ratios of the density of CCTs to that of humoral vessels are up to 289±6 folds (FIG. 37B). The above observations suggest that cytocapsular tumor metastasis via CCT freeway systems dominates tumor metastasis in vivo. Occasionally, a very low percentage of CCTs invade into micro blood vessels and release oncocells into the circulation systems (FIGS. 37C-37F), indicating that humoral vessel CCT invasion-caused oncocell release is a source of circulating tumor cells in the blood. Furthermore, we tested metastases of cytocapsular oncocells in 35 types of cancers (1~10 secondary niches per type), and observed that cytocapsular tumors broadly disseminate to multiple secondary niches (Table 6), which are consistent with clinical observations that primary tumors always metastasize into multiple tissues and organs.

### Example XVI

### Secondary Cytocapsular Tumor Network System Progression

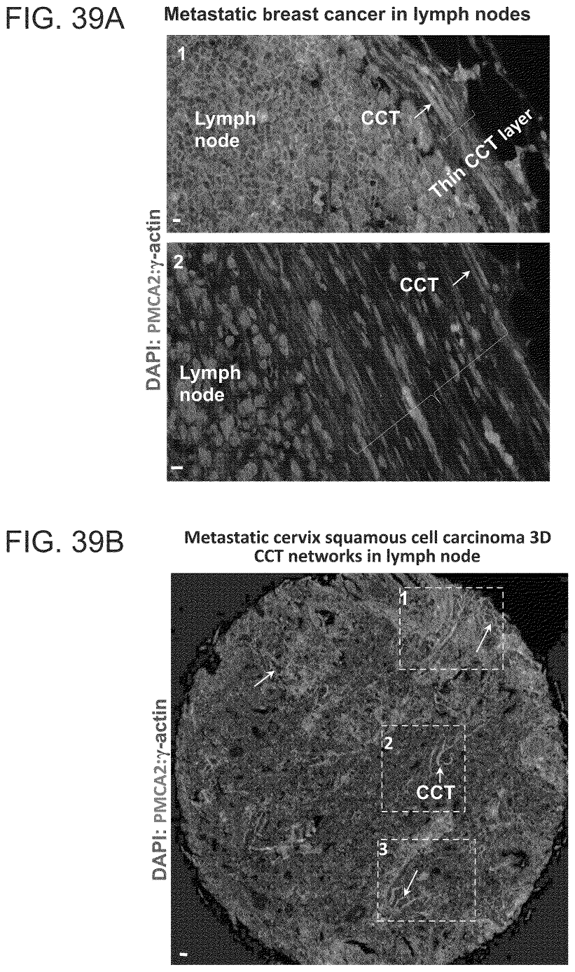

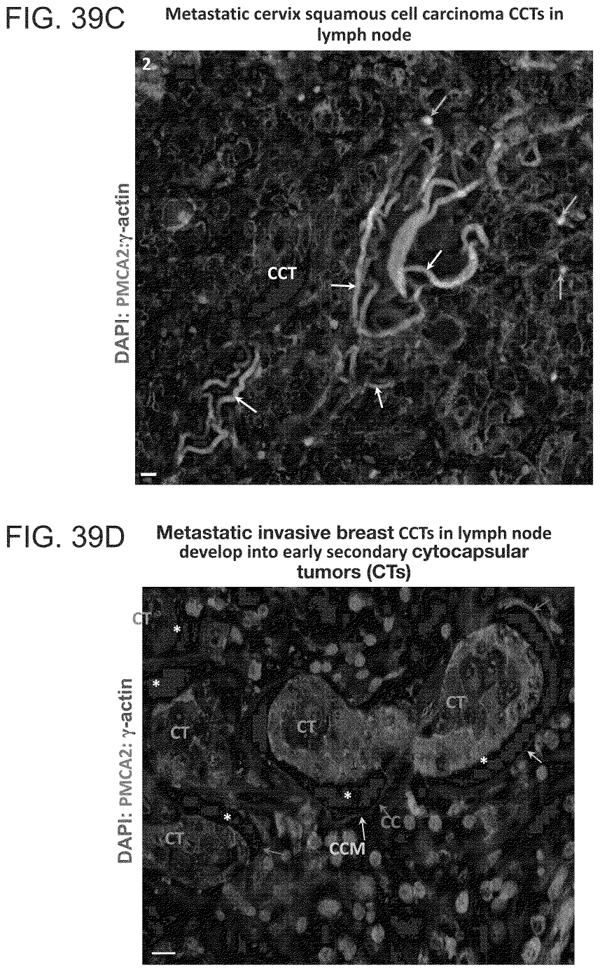

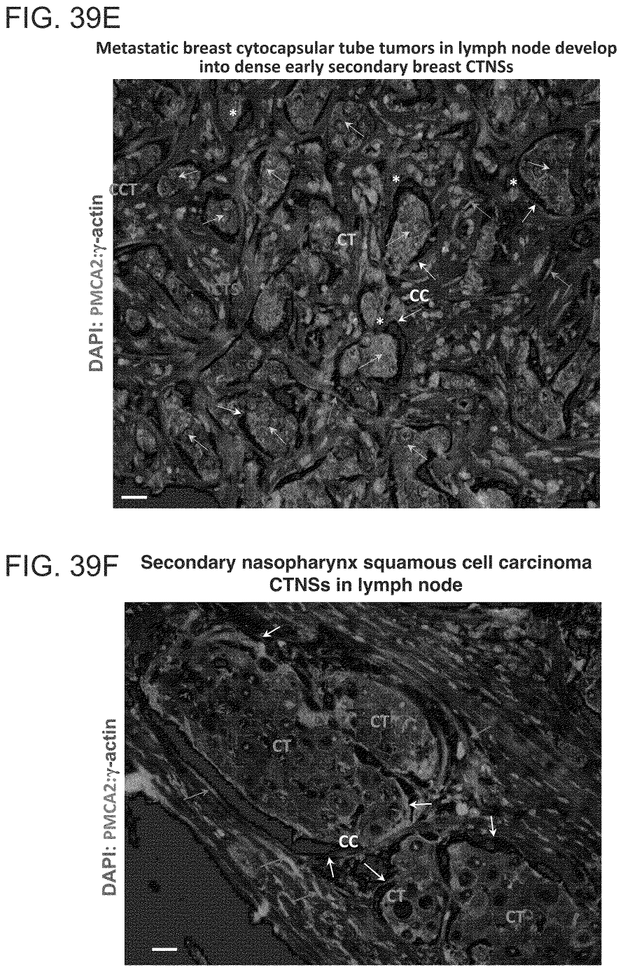

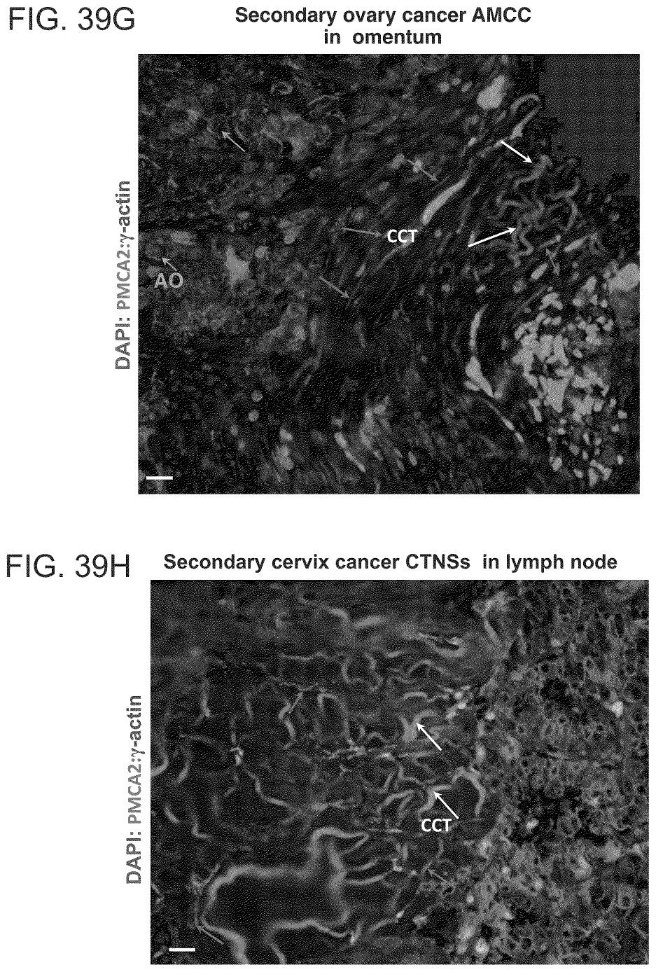

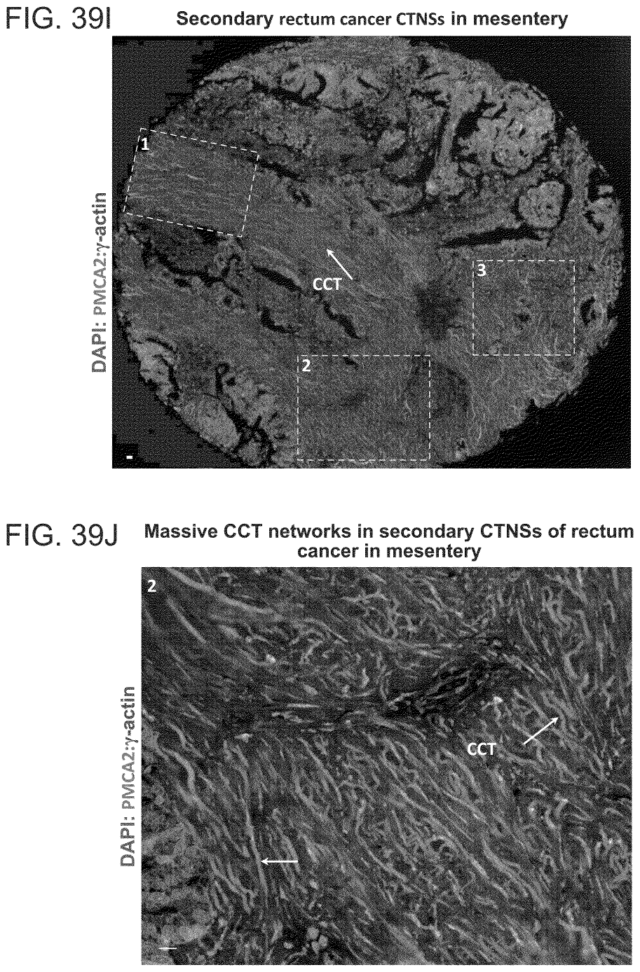

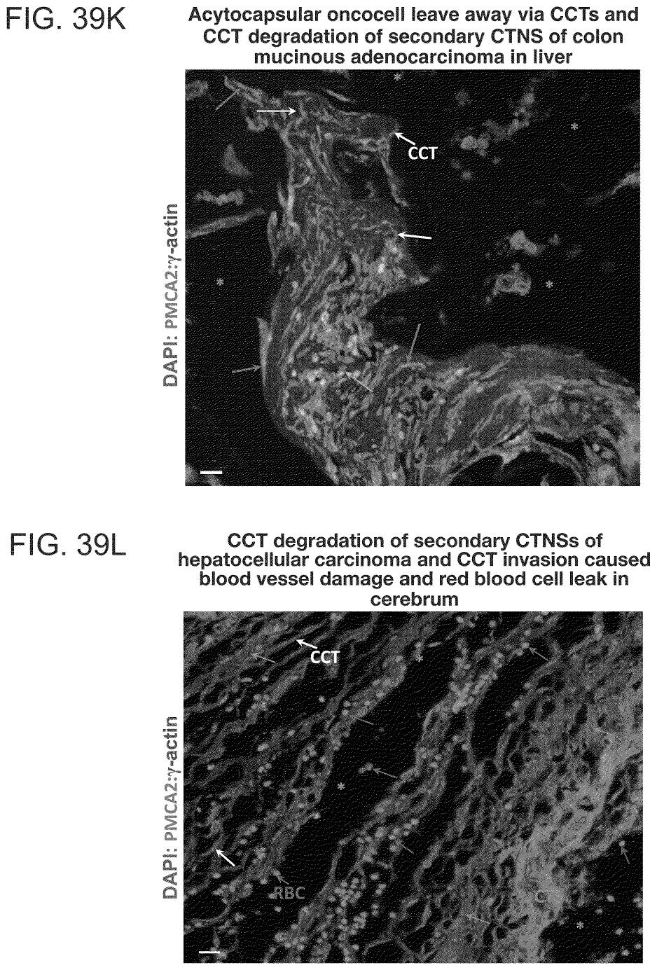

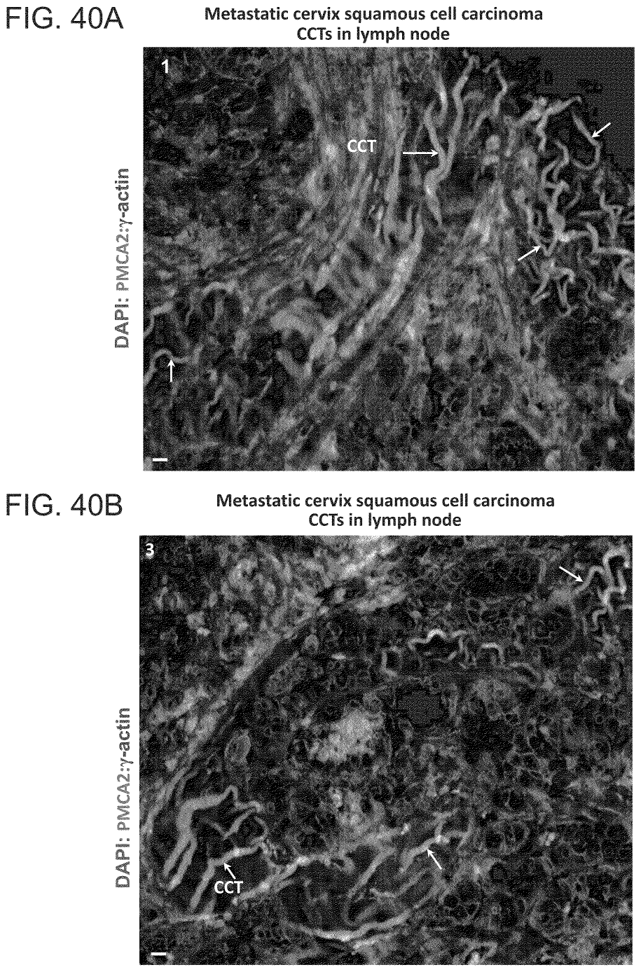

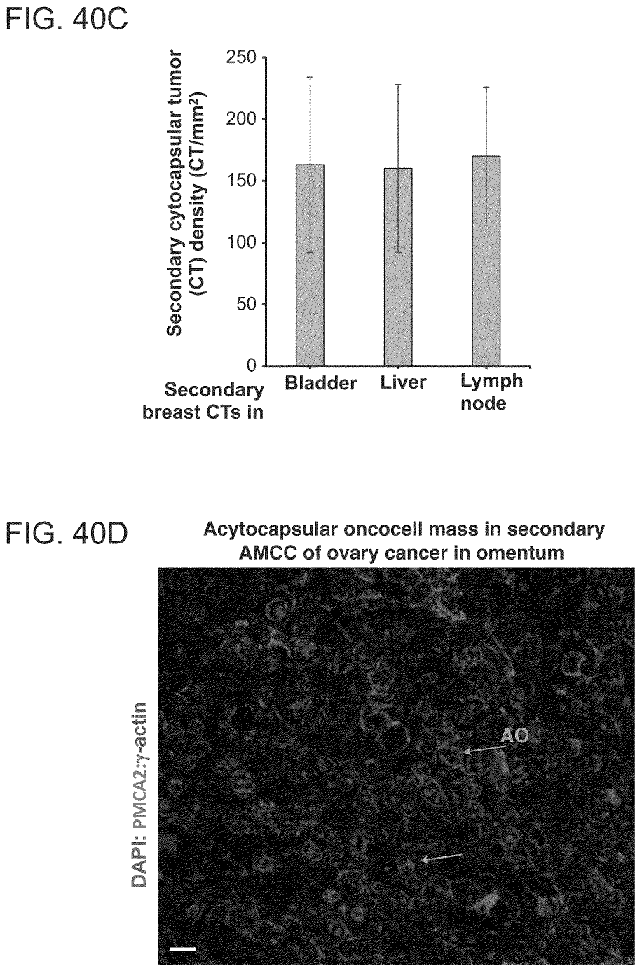

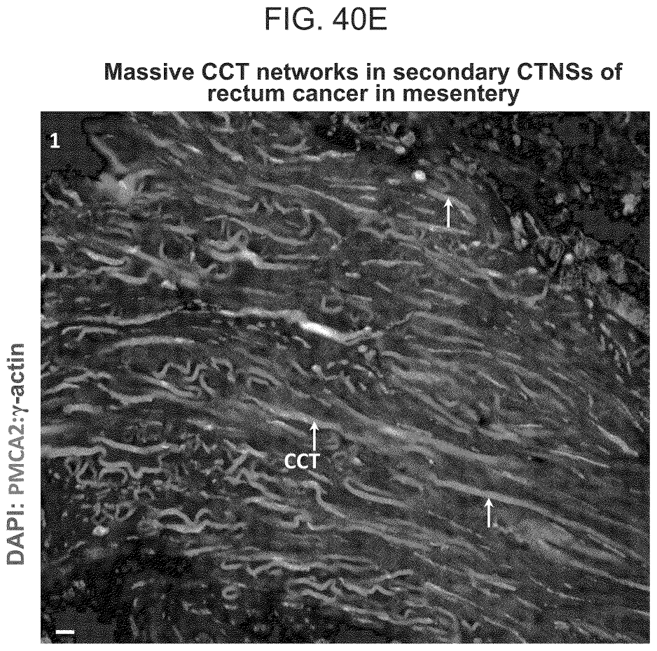

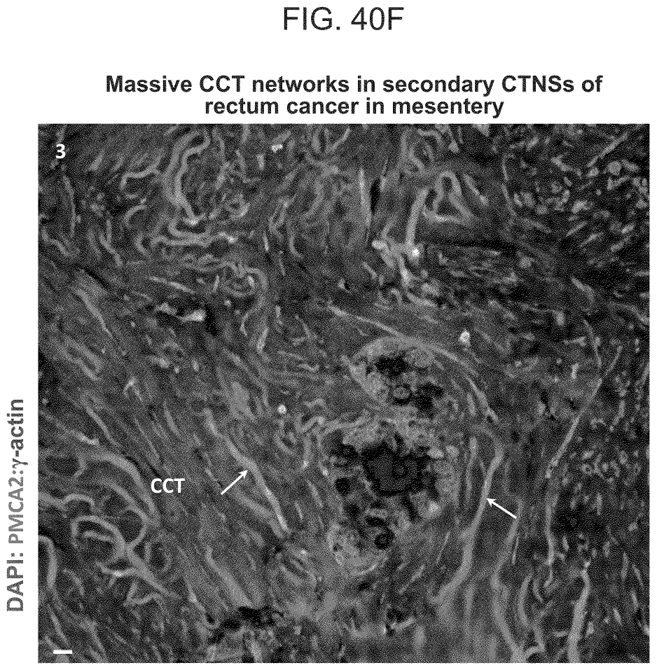

Cancer metastasis and secondary tumors caused tissues/organs biological function failure are major causes of cancer deaths (Gerstberger S, et al., Metastasis. Cell. 186, 564-1579 (2023); Hebert D, et al., Dissecting metastasis using preclinical models and methods. Nat Rev Cancer. 23, 391-407 (2023)). Thus, we assessed if metastasized cytocapsular oncocells in secondary niches generate secondary cytocapsular tumors and CTNSs in vivo. Indeed, after breast cytocapsular oncocell CCT networks metastasize and arrive at lymph nodes, they initially form thin CCT layers wrapping lymph nodes (FIG. 39A, panel 1). Subsequently, with CCT branching morphogenesis, more breast cytocapsular oncocell CCTs are generated and form much thicker CCT layers, enveloping lymph nodes (FIG. 39A, panel 2). Cervix cytocapsular oncocell CCTs massively invade into lymph nodes with compact lymphocytes, and a lot of metastasized-cervix cytocapsular oncocells disseminate into dense lymph nodes (FIGS. 39A-39C and FIGS. 40A-40B). Subsequently, in lymph nodes, metastasized breast cytocapsular oncocells in CCTs proliferate, and generate many small secondary breast cytocapsular tumors in enlarged CCs with high CT density (FIG. 39D). At early stage, secondary cytocapsular breast tumors exhibit C-shaped or irregular-shaped CCT lumen gaps (up to 30 μm in width) between the surface of cytocapsular oncocell masses and the CC membranes (FIGS. 39D-39E). Breast cytocapsular oncocells in CCTs metastasized at lymph nodes grow into large quantities of secondary breast cytocapsular tumors, which occupy the spaces of normal lymph node cells, and many normal lymph node cells disappeared (FIG. 39F). These secondary breast cytocapsular tumors are interconnected via CCT networks and form secondary breast CTNSs in the secondary niches (FIG. 39F). The average densities of the checked (examined) secondary breast cytocapsular tumors in bladder, liver and lymph node are 163±71 CTs/mm², 160±68 CTs/mm², and 170±56 CTs/mm², respectively (patients, n=4~6) (FIG. 40C). The above observations (FIGS. 39D-39F) indicate that the primary CTNSs are physically interconnected with secondary CTNSs via CCT networks and form integrated primary and secondary CTNSs. Small nasopharynx secondary cytocapsular tumors grow up and engender many CCTs (FIG. 39F). CCs and CCTs of ovary secondary cytocapsular tumors in omentum degrade, and generate big acytocapsular ovary tumors/oncocell masses (up to 2 cm or more in diameter/width) without CCs/CCTs (FIG. 39G and FIG. 40D). Subsequently, some oncocells in acytocapsular ovary tumors in omentum regenerate new CCTs CTs, and CNTSs (FIG. 39G). Consistently, some cervix oncocells of acytocapsular cervix tumors in lymph node engender many CCTs in lymph nodes (FIG. 39H), and rectum oncocells in acytocapsular rectum tumors in mesentery engender large quantities of new CCTs and networks in mesentery (FIGS. 39I-39J and FIGS. 40E-40F). Acytocapsular oncocells invade into the regenerated CCTs via alloentry and leave away (FIGS. 39G-39J). Many secondary colon acytocapsular oncocells in liver disseminate via regenerated colon CCTs, leaving many spaces without cells and only filled with intercellular fluids (FIG. 39K).

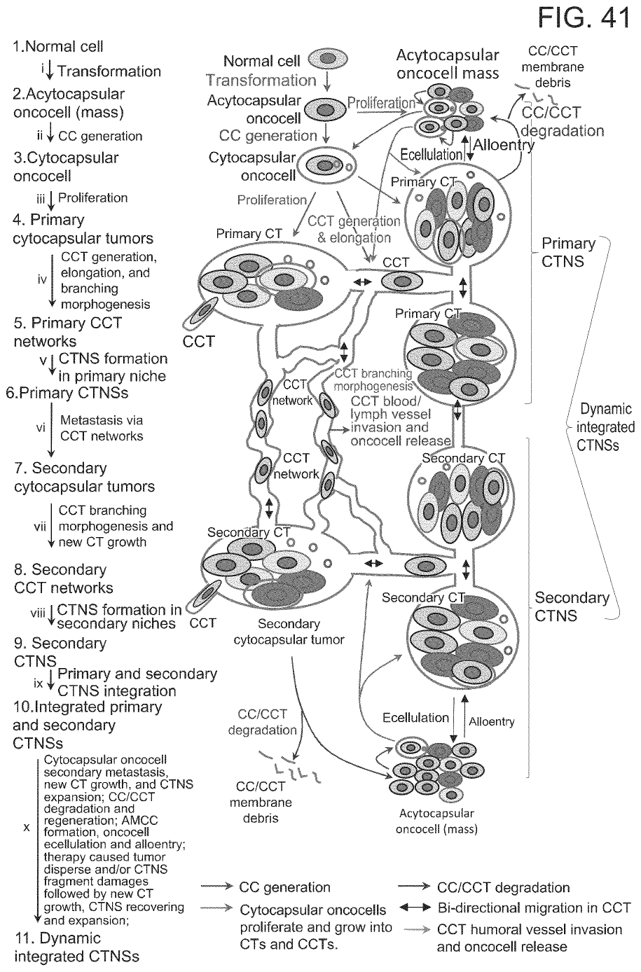

In the late cancer stage, after secondary dissemination of metastasized cytocapsular tumors via CCT networks, CCTs degrade, and CCT networks decompose (FIGS. 39K-39L). Sometimes, many red blood cells randomly spread in large areas in the secondary hepatocellular carcinoma in cerebrum of brain, indicting some (micro) blood vessels are broken and leaky caused by CCT invasion and (micro) blood vessel decomposition, and red blood cells are released (FIG. 39L). The above observations suggest that metastatic cytocapsular tumor progression in the secondary niches includes 6 major successive stages: 1) arrival and invasion of metastatic cytocapsular oncocells in CCTs in the secondary niches, 2) generation of secondary cytocapsular tumors, 3) formation of secondary CTNSs, 4) formation of dynamic integrated primary and secondary CTNSs via CCT networks, 5) CC/CCT degradation and formation of AMCC, 6) generation of new CCTs for oncocells' next metastasis (FIG. 39 and FIG. 40). These observations (Table 6, FIGS. 37 and 39 and FIGS. 35, 36, 38 and 40) suggest that metastasized cytocapsular oncocells have capacities to generate large quantities of small/middle-sized secondary cytocapsular tumors and dense CTNSs in the secondary niches (in neighboring or far-distance organs/tissues), and lead to massive normal cell disappearance followed by affected, harmed, or even failed biological functions in the secondary niches related tissues/organ. In summary, our results suggest that cytocapsular oncocells, cytocapsular tumors, and integrated primary and secondary CTNSs coordinate membrane-sheltered cancer progression in human (FIG. 41 and Table 7).

TABLE 7
Advantages and disadvantages of CC/CCT, CT and CTNS in comparison to other
organelles, compartments and cellular activities beyond CC/CCT in vivo.

| | | | | | Cancer cell |
|---|---|---|---|---|---|
| | | | Nanotube | Type II | activities |
| | | CC, CCT, | (Types I | epithelial | beyond |
| | Characters | CT, CTNS | and II) | bridge | CC/CCT |
| 1 | Uncontrolled cell proliferation inside | Yes | No | No | No |
| | the organelle/compartment | | | | |
| 2 | Cell migration inside the | Yes | No | No | No |
| | organelle/compartment | | | | |
| 3 | Protected cell migration without | Yes | No | No | No |
| | obstacles of heterogeneous ECM and | | | | |
| | neighboring cells | | | | |
| 4 | Protected cell translocation with | Yes | No | No | No |
| | immune attack escape | | | | |
| 5 | Increased pan-drug resistance by | Yes | No | No | No |
| | extracellular membrane protective | | | | |
| | barriers | | | | |
| 6 | Bi-directional oncocell translocation | Yes | No | No | No |
| | between neighboring or long-distance | | | | |
| | tumors via CCT networks | | | | |
| 7 | Increased survival ability by elevated | Yes | No | No | No |
| | nutrient taking capacities of integrated | | | | |
| | primary tumor network systems | | | | |
| 8 | Increased survival ability by elevated | Yes | No | No | No |
| | nutrient taking capacities of integrated | | | | |
| | secondary tumor network systems | | | | |
| 9 | Increased survival ability by elevated | Yes | No | No | No |
| | nutrient taking capacities of integrated | | | | |
| | primary and secondary tumor networks | | | | |
| 10 | Ecellulation, auto-entry and alloentry | Yes | No | No | No |
| | provide membrane-enclosed protected | | | | |
| | environments for cellular activities | | | | |
| 11 | Protected oncocell dissemination in | Yes | No | No | No |
| | membrane-enclosed tube-shaped | | | | |
| | freeway systems | | | | |
| 12 | Cancer bone metastasis via CCT | Yes | No | No | No |
| | invasion into hard tissue | | | | |
| 13 | Cancer brain metastasis via CCT | Yes | No | No | No |
| | invasion through blood-brain barrier | | | | |
| 14 | Wrapping tumors with multiple and | Yes | No | No | No |
| | many CCT protection layers | | | | |
| 15 | Form CCT superstructures in cancer | Yes | No | No | No |
| | tissues for massive CCT invasion in | | | | |
| | tissues with heterogeneous densities | | | | |
| | and textures | | | | |
| 16 | Harbor many CCTs and oncocells in | Yes | No | No | No |
| | normal tissues adjacent tumor tissues | | | | |
| | (NAT) | | | | |
| 17 | Regeneration of CC membranes and | Yes | No | No | No |
| | CCTs in multiple times | | | | |
| 18 | Generate large 3D networks | Yes | No | No | No |
| 19 | Upon chemotherapy, tumor cells are | Yes | No | No | No |
| | dispersed into other sites in membrane- | | | | |
| | enclosed systems followed by tumor | | | | |
| | relapse and cancer survival | | | | |
| 20 | Generate extracellular bio-membrane | Yes | No | No | No |
| | systems beyond circulation systems and | | | | |
| | present immune attack/therapy cold | | | | |
| 21 | Dynamic extracellular biomembrane | Yes | No | No | No |
| | systems protect oncocell activities and | | | | |
| | behaviors inside | | | | |
| 22 | Extracellular biomembrane system | Yes | No | No | No |
| | generation, degradation and | | | | |
| | regeneration protect tumor progression | | | | |
| | and evolution | | | | |
| 23 | Additional membrane barrier decreases | Yes | No | No | No |
| | efficiency of nutrient molecule taking | | | | |
| | and metabolic waste molecule diffusion | | | | |
| 24 | Additional membrane generation | Yes | No | No | No |
| | increases energy consuming | | | | |
| 25 | Introduce tissue structure interference | Yes | | | Yes |
| | and damage and biological function | | | | |
| | failure into normal tissues | | | | |

The present disclosure provides observations and results that show that CCs and CCTs are universally present in cancers in vivo. The PMCA2 calcium pump was identified as the most upregulated factor in malignant tumors, but was found in very low abundance in healthy tissues. This conclusion was based on interrogating more than 10,000 samples from cancer tissue banks worldwide.

The present disclosure provides new mechanistic insights that are listed below, which dramatically widened our understanding of cancer malignancy.
(i) Discoveries of cytocapsular oncocell, cytocapsular tumor, cytocapsulasome, cytocapsular tumor network system (CTNS), integrated primary and secondary CTNSs.
(ii) All malignant tumors engender a second extracellular membrane, reminiscent of ancient forms of life, such as mitochondria, chloroplast and gram-negative bacteria. The presence of the PMCA2 calcium pump in the second extracellular membrane is essential for malignancy for all known cancers. Absence of high levels of PMCA2 defines tissues as benign, non-malignant and normal. This is a potent tool for cancer diagnosis and therapy.
(iii) PMCA2 has been validated as marker for malignant cancer by the FDA (available on FDA website, 2020)
(iv) PMCA2-containing cytocapsulasome vesicles appear to promote CCT elongation, contribute to tumor progression and invasion even into solid tissue or hard trabecular bone.
(v) Multicellular malignant tumors are surrounded by CC membranes.
(vi) Alloentry allow acytocapsular oncocells invade into CCTs followed by CCT-directed metastasis; Ecellulation of large CCs creates cell-less scaffolding structures.
(vii) CCT mediated disposal of tumor cells and normal cells creates empty liquid filled holes rendering tissue non-functional (FIG. 34).
(viii) Initial metastases involve transport of tumor cells through CCTs, which only later penetrate humoral vessels for cancer dissemination by previously known pathways.
(ix) Secondary cytocapsular network systems use CCTs for dissemination of multi-cell cancer dissemination.
(x) The cytocapsular oncocell, cytocapsular tumor, cytocapsular oncocell metastasis in CCT networks, and cytocapsular tumor network systems in cancer development and progression, which may facilitate the researches for effective therapies against cancers.

The discoveries of progression of cytocapsular oncocells and CTs, CTNSs, and integrated primary and secondary CTNSs in human organs/tissues described in the present disclosure may facilitate further cancer research, early screening, prognosis, diagnosis, drug development, therapy and treatment. Extended analysis of the proteome and metabolome of CCTs of multiple cancer types may provide additional insights into cancer mechanisms and yield new targets for cancer diagnosis and therapy (in progress), The time course and causality of cancer progression suggested here was mostly based on comparing static snap shots from a large number of tissues samples that are consistent with mechanisms of progress.

The discoveries of progression of cytocapsular oncocells and CTs, CTNSs, and integrated primary and secondary CTNSs in human organs/tissues described in the present disclosure may facilitate further cancer research, early screening, prognosis, diagnosis, drug development, therapy and treatment.

### Example XVII

### Additional Materials and Methods

### Reagent, Antibodies and Devices

CC/CCT culture kits (Celldevi, avaible at Celldevi website www.celldevi.com, Cat. CD0104, 6-well plates; Cat. CD0105, 12-well plates, and Cat. CD 0106, 24-well plates) and kits with glass cover slips in the well bottom and embedded by CC/CCT culture matrix layer (Cat. CD 0112, 6-well plate) were ordered from Celldevi Inc. CC/CCT culture kit fixation kit (Cat. CD0201) were ordered from Celldevi Inc. Cancer cell lines of pancreas cancer cell Bxpc3, breast cancer cell MCF-7 and colon cancer cell SK-CO-1 and cell culture media were ordered from ATCC. ¹³C₆, ¹⁵N₂-L-Lysine (Cat. 88209) and ¹³C₆, ¹⁵N₄-L-Arginine (Cat. 89990) for SILAC labeling were ordered from Thermo Fisher Scientific. Unipick™ and capillary units were ordered from NeuroInDx Inc. Rabbit anti-PMCA2 antibody (polyclonal, ab3529; 1:200 dilution), mouse anti-γ-actin antibody (monoclonal, ab123034; 1:200 dilution) for immunofluorescence assay were ordered from Abcam. DAPI (1:1,000 dilution in the immunofluorescence assay) was ordered from KPL. Human normal and cancer tissue specimens were ordered from TissueArray and US Biolab (or gifted by local hospitals).

### Stable SILAC Labeled CC/CCT Culture and Collection for CC/CCT Proteome Analyses

Following CC/CCT culture kit usage instructions, stable SILAC labeled cancer cells of Bxpc3, MCF-7 and SK-CO-1 with ¹³C₆, ¹⁵N₂-L-Lysine and ¹³C₆, ¹⁵N₄-L-Arginine were implanted in CC/CCT culture kits (6-well plates) with cell culture media with ¹³C₆, ¹⁵N₂-L-Lysine and ¹³C₆, ¹⁵N₄-L-Arginine¹. Stable SILAC labeled cancer cells generate stable SILAC labeled cytocapsulas (CCs) and cytocapsular tubes (CCTs). Sometimes, some incytocapsular oncocells are spontaneously evicted from CCs and CCTs in vitro. After ecellulation, evicted cancer cells were washed away by three washes with 1×PBS. Acellular CCs/CCTs (ECC/ECCTs) were collected by Unipick and kept on ice followed by storage at −80 °C. More than 400,000 stable SILAC labeled acellular CCs/CCTs per CC/CCT proteome analysis sample were collected in >4 years.

### Cytocapsular Tumorsphere and Cytocapsula Growth In Vitro, Immunohistochemistry Staining and Imaging

Pancreas cancer Bxpc3 cells were implanted in CC/CCT culture kit (Cat. CD 0112, Celldevi) following the kit manual. At 36 h, Bxpc3 cells generated cytocapsulas (CCs). Some cytocapsular oncocells performed ecellulation. Cytocapsular oncocells and ecellulated CCs were performed fixation kit and immunohistochemistry staining. At different time of 48 h, 72 h, 68 h, 74 h, 78 h, 84 h, 96 h, 108 h after cell implantation, Bxpc3 cancer cells engender cytocapsular oncocells, and grow into cytocapsular tumorspheres in different sizes with CC tightly wrapping oncocell mass or with wide cytocapsular lumens, and ecellulation of cytocapsular tumorspheres. These cytocapsular tumorspheres and ecellulated cytocapsular tumorspheres were fixed by Celldevi Inc. CC/CCT fixation kit (Celldevi, CD0201) in the 6-well plate, and then taken out and put onto slides, followed by immunohistochemistry (IHC) staining.

### Liquid Chromatography Tandem Mass Spectrometry CC/CCT Proteome Analyses

The proteins in collected ECCs/CCTs with stable SILAC labeled with ¹³C₆, ¹⁵N₂-L-Lysine and 13C₆, ¹⁵N₄-L-Arginine were extracted and purified by SDS-gel electrophoresis. After Coomassie blue staining, the SDS-gel strip of one sample was cut into 4-5 gel slices. After in-gel digestion 12.5 ng/μL trypsin, the digested peptides were extracted and enriched. The enriched peptides were used for liquid chromatography tandem mass spectrometry (LC-MS/MS) analyses as previously described (Everley A, et al., Quantitative cancer proteomics: stable isotope labeling with amino acids in cell culture (SILAC) as a tool for prostate cancer research. Mol Cell Proteomics. 3, 729-35 (2004)). The enriched peptide fractions were analyzed by liquid chromatography tandem mass spectrometry (LC-MS/MS) on an LTQ Orbitrap Velos mass spectrometer (Thermo Scientific) equipped with a Thermo Fisher Scientific nanospray source, an Agilent 1100 Series binary HPLC pump, and a Famos autosampler. Peptides were separated on a 0.125×180 mm fused silica microcapillary column with an in needle tip (made in-house) with a ~5-μm i.d. The silica microcapillary column was packed with magicC18AQ C18 reverse-phase resin (5-μm particle size, 200-Å pore size; Michrom Bioresources). Separation was performed by applying a 57-min gradient from 7% to 28% acetonitrile in 0.125% formic acid. The mass spectrometer was operated with default settings: full MS [automatic gain control (AGC), 1×10⁶; resolution, 6×10⁴; m/z range, 375-1,800; maximum ion time, 1,000 ms]; MS/MS (AGC, 5×10³; maximum ion time, 120 ms; minimum signal threshold, 4×10³; dynamic exclusion time setting, 30 s; charged ions and ions for which no charge state could be determined were excluded MS/MS selection). Triplicated independent experiments were performed.

### Database Searches, Data Filtering, Validation of Protein Detection Rate, and Proteome Analyses.

The spectral data were searched with a tandem mass spectrometry data analysis program sold under the trademark SEQUEST™ against a database containing the human protein sequence database (available from ensembl.org website) together with the reversed complement. The LC-MS/MS identifications were filtered to 0.98% protein false discovery rate (FDR) and 0.1% peptide FDR. The peptide quantification and phosphorylation site localization were analyzed using in-house software and Ascore as previously described (Yi T, et al., Quantitative phosphoproteomic analysis reveals system-wide signaling pathways downstream of SDF-1/CXCR4 in breast cancer stem cells. Proc Natl Acad Sci USA. 111, E2182-90 (2014)).

### Cytocapsular Tumorsphere and Cytocapsula Growth In Vitro, Immunohistochemistry Staining and Imaging

IHC staining was performed with rabbit anti-PMCA2 polyclonal primary antibodies (1:200 dilution), mouse anti-γ-actin monoclonal primary antibodies (1:200 dilution), Goat anti-Mouse IgG (H+L) Highly Cross-Adsorbed Secondary Antibody, Alexa Fluor Plus 555 (Thermo Fisher), and Goat anti-Rabbit IgG (H+L) Highly Cross-Adsorbed Secondary Antibody, Alexa Fluor Plus 488, Thermo Fisher), and DAPI staining (1:000 dilution). Fluorescence images were taken with a Nikon 80i upright microscope with a 20× or 40× lens. All images were obtained using MetaMorph image acquisition software and were analyzed with ImageJ software.

### CCT Histology and Immunohistochemical Staining Analysis

The 9972 formalin-fixed, paraffin-embedded (FFPE) human cancer tissue specimens (4-5 μm in thickness) from 9784 cancer patients, 14 human normal tissue FFPE specimens form 14 patients, and 126 human benign tumor tissue FFPE specimens from 126 patients were processed immunohistochemistry and hematoxylin and eosin (H&E) staining. Immunohistochemical fluorescence tests were performed to stain cytocapsular tubes using rabbit anti-PMCA2 polyclonal primary antibodies (1:200 dilution), mouse anti-γ-actin monoclonal primary antibodies (1:200 dilution), Goat anti-Mouse IgG (H+L) Highly Cross-Adsorbed Secondary Antibody, Alexa Fluor Plus 555 (Thermo Fisher), and Goat anti-Rabbit IgG (H+L) Highly Cross-Adsorbed Secondary Antibody, Alexa Fluor Plus 488, Thermo Fisher), and DAPI staining (1:000 dilution). Fluorescence images were taken with a Nikon 80i upright microscope with a 20× or 40× lens. All images were obtained using MetaMorph image acquisition software and were analyzed with ImageJ software.

### Time-Lapse DIC Microscopy and Videos

Time-lapse DIC microscopy analyses of cytocapsula elongation and cell migration were performed using a Nikon Ti motorized inverted microscope and a digital Hamamatsu ORCA-ER cooled CCD camera with a 20× lens. The time-lapse microscope was equipped with DIC, phase contrast, and epi-fluorescence optics, a Prior ProScan III mo-torized stage and shutters, a perfect focus system, and an Okolab 37 °C, 5% CO₂ cage microscope incubator (Okolab). Images were taken every 30 s over the course of ~10-36 h. All images were obtained using MetaMorph software. Tracks made by 2 h of cytocapsula elongation were obtained using MetaMorph and ImageJ software. Cytocapsula elongation velocities were also calculated using length and time measurements. Movies were prepared using the images collected via time-lapse and Meta-Morph software (15 frames/s).

### Bright Filed Microscope and Videos

Bright field microscope analyses of cytocapsula growth with cytocapsulasomes activities were performed using Nikon Eclipse TS2 Inverted Routine Microscope with a DS-FI3 Microscope Camera with a phase contrast 20× lens. The videos were taken using NIS-Elements software (25 fps, frame per second).

### Imaging Acquisition

DIC and fluorescence images of fixed cells (with or without cytocapsulae) were taken with an 80i upright microscope and a digital Hamamatsu ORCA-ER cooled CCD camera with a 20× or 40× lens. The bright-field phase-contrast image was taken using a Nikon digital camera. The cytocapsula initiation ratio per high-performance field (HPF; 200×) and the number of elongated cytocapsulae per high-performance field were quantified. All images were obtained using MetaMorph image acquisition software and were analyzed with ImageJ software.

### Data Collection

Cytocapsular tubes (CTs, not sectioned, longitudinally sectioned, and cross sectioned) without degradation (3~6 μm in measured diameter) were counted using a fluorescence microscope and ImageJ. The presence of CTs degrading into thick strands (1~2 μm in measured diameter), thin strands (0.2~1 μm in measured diameter), or the disintegration state were reported without quantification. The patients providing formalin-fixed paraffin-embedded tissue samples gave informed consent that they understood that the biopsies (needle biopsy or surgical biopsy, from US Biomax) were performed for in vitro research purposes only. Comparative deidentified samples of normal tissues, benign tissues, carcinoma in situ, cancer, paracancer, metastatic tissues with their cancer stages identified according to the tumor (T), node (N), and metastasis (M) TNM system (cancer stages: 0, I, II, III, IV) were obtained from archival materials. The cancer, paracancer and metastatic cancer tissues, in which the original cancer niches were identified by indicated cancer specific molecular markers, were identified by hospital pathology laboratories and obtained from archival material. Autopsy tissues samples have many post-life CC/CCT degradation and CCs/CCTs will not be quantified. Biopsy samples from FFPE with fresh tissues present high fidelity of CC/CCT status and CCs/CCTs are quantified and reported.

### Additional Quantification and Statistical Analysis

The statistical methods used for comparisons are indicated in the relevant figure legends and in the sections below. The diameters, widths, and lengths of cytocapsulae and cytocapsular tubes were measured with MetaMorph or ImageJ. The time of individual cytocapsulae and cytocapsular tubes was counted from cytocapsula generation to acellular cytocapsula (or cytocapsular tube) decomposition. For lifetime of cytocapsular oncocell and cytocapsular tumorsphere assays, at least 20 cytocapsular oncocell or cytocapsular tumorsphere were measured per condition, and two-tailed Student's test was used to determine statistical significance. The video taken time was labeled as hour: minute: second (in FIGS. 28A and 29A), while times after cell implantation were 96 h in FIG. 28 and 108 h in FIG. 29. In FIG. 31A, the video taken time is just after cell implantation. The graph plots are mean±SD. In CCT analysis in cancer types/subtypes, at least 3 samples per cancer subtype were checked. In cytocapsular tube quantitation assays, for each specimen, the number of fully intact cytocapsular tubes was counted in 5 areas (0.35 mm×0.35 mm, length×width) of the sample (top, bottom, left, right, and center), and the cytocapsular tube density (CCT/mm²) was calculated and determined for each area. The average CCT density across the 5 sites was treated as the specimen's overall CCT density and round up to digits. The quantitation of a humoral vessel density employs the similar method as CCT quantitation.

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application was specifically indicated to be so incorporated by reference.

1. A method of detecting cytocapsular tubes in a tissue of a subject, comprising
a) obtaining a tissue from the subject, and
b) detecting the presence of the cytocapsular tubes in the tissue by contacting the tissue with an anti-plasma membrane Ca²⁺-ATPase (PMCA) antibody.

2. The method of claim 1 wherein the subject is a mammal.

3. The method of claim 1 wherein the subject is a human or an animal.

4. The method of claim 1 wherein the subject is healthy or diseased.

5. The method of claim 1 wherein the subject is suffering from cancer or suspected to suffer from cancer.

6. The method of claim 1 wherein the tissue is obtained from anywhere in the body of the subject.

7. The method of claim 1 wherein the tissue comprises healthy, benign, or cancerous tissue.

8. The method of claim 7 wherein the cancerous tissue comprises any tumor tissue comprising the primary site of tumor origin or the secondary site of tumor metastasis.

9. The method of claim 7 wherein tissue adjacent to the cancerous tissue is obtained.

10. The method of claim 1 wherein the tissue is obtained by biopsy.

11. The method of claim 10 wherein the biopsy comprises clinical biopsy, bone marrow aspiration and biopsy, cardiac biopsy, core biopsy, endometrial biopsy, endoscopic biopsy, excisional and incisional biopsy, fine-needle aspiration biopsy, lymph node biopsy, needle biopsy, open biopsy, punch biopsy, sentinel lymph node biopsy, shave biopsy, skin biopsy and the like.

12. The method of claim 1 wherein the PMCA comprises PMCA-1, PMCA-2, PMCA-3 and PMCA-4.

13. The method of claim 1 wherein the antibody comprises anti-PMCA-1 antibody, anti-PMCA-2 antibody, anti-PMCA-3 antibody, and anti-PMCA-4 antibody.

14. A method of predicting the grade of cancer metastasis of a subject, comprising
a) obtaining a tissue from the subject, and
b) detecting the presence of the cytocapsular tubes in the tissue by contacting the tissue with an anti-plasma membrane Ca²⁺-ATPase (PMCA) antibody.

15. The method of claim 14 wherein the presence of the cytocapsular tubes in the tissue is associated with metastasis cancer.

16. The method of claim 14 wherein the density and morphology of the cytocapsular tubes detected in the tissue is used to predict the grade of cancer metastasis of the subject.

17. The method of claim 14 wherein increase in the density of cytocapsular tubes is associated with more advanced grade of cancer metastasis.

18. The method of claim 14 wherein thinner and cloud-like cytocapsular tube morphology and more degradation is associated with more advanced grade of cancer metastasis.

19. The method of claim 14 wherein if no cytocapsular tube (CCT) is detected and no CCT degradation is detected, cancer metastasis (CM) of grade 0 is predicted.

20. The method of claim 14 wherein if the density of CCT detected is in the range of 1-10/mm² and no CCT degradation is detected, cancer metastasis (CM) of grade 1 is predicted.

21. The method of claim 14 wherein if the density of CCT detected is in the range of 11-40/mm², and CCTs are degraded into strands and thin strands, cancer metastasis (CM) of grade 2 is predicted.

22. The method of claim 14 wherein if the density of CCT detected is in the range of 41-80/mm², and CCTs are degraded into strands, thin strands, and silk-like strands, cancer metastasis (CM) of grade 3 is predicted.

23. The method of claim 14 wherein if the density of CCT detected is in the range of >81/mm², and CCTs are degraded into cloud-like morphology or completely decomposed, cancer metastasis (CM) of grade 4 is predicted.

24. The method of claim 14 wherein the subject is a mammal.

25. The method of claim 14 wherein the subject is a human or an animal.

26. The method of claim 14 wherein the subject is healthy or diseased.

27. The method of claim 14 wherein the subject is suffering from cancer or suspected to suffer from cancer.

28. The method of claim 14 wherein the tissue is obtained from anywhere in the body of the subject.

29. The method of claim 14 wherein the tissue comprises healthy, benign, or cancerous tissue.

30. The method of claim 29 wherein the cancerous tissue comprises any tumor tissue comprising the primary site of tumor origin or the secondary site of tumor metastasis.

31. The method of claim 29 wherein tissue adjacent to the cancerous tissue is obtained.

32. The method of claim 14 wherein the tissue is obtained by biopsy.

33. The method of claim 32 wherein the biopsy comprises clinical biopsy, bone marrow aspiration and biopsy, cardiac biopsy, core biopsy, endometrial biopsy, endoscopic biopsy, excisional and incisional biopsy, fine-needle aspiration biopsy, lymph node biopsy, needle biopsy, open biopsy, punch biopsy, sentinel lymph node biopsy, shave biopsy, skin biopsy and the like.

34. The method of claim 14 wherein the PMCA comprises PMCA-1, PMCA-2, PMCA-3 and PMCA-4.

35. The method of claim 14 wherein the antibody comprises anti-PMCA-1 antibody, anti-PMCA-2 antibody, anti-PMCA-3 antibody, and anti-PMCA-4 antibody.

36. A method of screening a subject for cancer, comprising
a) obtaining a tissue from the subject, and
b) detecting the presence of the cytocapsular tubes in the tissue by contacting the tissue with an anti-plasma membrane Ca²⁺-ATPase (PMCA) antibody.

37. The method of claim 36 wherein the presence of the cytocapsular tubes in the tissue is an indication that the tissue is cancerous.

38. The method of claim 36 wherein the absence of the cytocapsular tubes in the tissue is an indication that the tissue is healthy or benign.

39. The method of claim 36 wherein the subject is a mammal.

40. The method of claim 36 wherein the subject is a human or an animal.

41. The method of claim 36 wherein the subject is healthy or diseased.

42. The method of claim 36 wherein the subject is suffering from cancer or suspected to suffer from cancer.

43. The method of claim 36 wherein the tissue is obtained from anywhere in the body of the subject.

44. The method of claim 36 wherein the tissue comprises healthy, benign, or cancerous tissue.

45. The method of claim 44 wherein the cancerous tissue comprises any tumor tissue comprising the primary site of tumor origin or the secondary site of tumor metastasis.

46. The method of claim 44 wherein tissue adjacent to the cancerous tissue is obtained.

47. The method of claim 36 wherein the tissue is obtained by biopsy.

48. The method of claim 47 wherein the biopsy comprises clinical biopsy, bone marrow aspiration and biopsy, cardiac biopsy, core biopsy, endometrial biopsy, endoscopic biopsy, excisional and incisional biopsy, fine-needle aspiration biopsy, lymph node biopsy, needle biopsy, open biopsy, punch biopsy, sentinel lymph node biopsy, shave biopsy, skin biopsy and the like.

49. The method of claim 36 wherein the PMCA comprises PMCA-1, PMCA-2, PMCA-3 and PMCA-4.

50. The method of claim 36 wherein the antibody comprises anti-PMCA-1 antibody, anti-PMCA-2 antibody, anti-PMCA-3 antibody, and anti-PMCA-4 antibody.

51. A method of detecting cytocapsular tubes in a tissue sample from a subject, comprising
a) contacting the tissue sample with an antibody, wherein the antibody binds to plasma membrane Ca²⁺-ATPase (PMCA) in cytocapsular membrane of the cytocapsular tubes, and
b) detecting the presence of the cytocapsular tubes in the tissue sample by detecting the antibody.

52. The method of claim 51 wherein the subject is a mammal.

53. The method of claim 51 wherein the subject is a human or an animal.

54. The method of claim 51 wherein the subject is healthy or diseased.

55. The method of claim 51 wherein the subject is suffering from cancer or suspected to suffer from cancer.

56. The method of claim 51 wherein the tissue sample is obtained from anywhere in the body of the subject.

57. The method of claim 51 wherein the tissue sample comprises healthy, benign, or cancerous tissue.

58. The method of claim 57 wherein the cancerous tissue comprises any tumor tissue comprising the primary site of tumor origin or the secondary site of tumor metastasis.

59. The method of claim 57 wherein the tissue sample comprises tissue adjacent to the cancerous tissue.

60. The method of claim 51 wherein the tissue sample is obtained by biopsy.

61. The method of claim 60 wherein the biopsy comprises clinical biopsy, bone marrow aspiration and biopsy, cardiac biopsy, core biopsy, endometrial biopsy, endoscopic biopsy, excisional and incisional biopsy, fine-needle aspiration biopsy, lymph node biopsy, needle biopsy, open biopsy, punch biopsy, sentinel lymph node biopsy, shave biopsy, skin biopsy and the like.

62. The method of claim 51 wherein the PMCA comprises PMCA-1, PMCA-2, PMCA-3 and PMCA-4.

63. The method of claim 51 wherein the antibody comprises an anti-PMCA-1 antibody, an anti-PMCA-2 antibody, an anti-PMCA-3 antibody, and an anti-PMCA-4 antibody.

64. The method of claim 51 wherein the antibody comprises a detectable label and the detecting includes detecting the detectable label.

65. The method of claim 51 wherein the antibody is a primary antibody and wherein the primary antibody is detected by a secondary antibody that binds to the primary antibody.

66. The method of claim 51 wherein the antibody is a polyclonal or a monoclonal antibody.

67. The method of claim 51 further comprises detecting cytocapsulas, cytocapsulasome vesicles, cytocapsular oncocells, cytocapsular tumorspheres, and cytocapsular tumorsphere network systems.

68. The method of claim 51 wherein the presence of the cytocapsular tubes in the tissue sample indicates cancer metastasis.

69. A method of detecting cytocapsular tubes in vitro, comprising
a) contacting the cytocapsular tubes in a 3D matrix culture with an antibody, wherein the antibody binds to plasma membrane Ca²⁺-ATPase (PMCA) in cytocapsular membrane of the cytocapsular tubes, and
b) detecting the presence of the cytocapsular tubes in the 3D matrix culture by detecting the antibody.

70. The method of claim 69 wherein the PMCA comprises PMCA-1, PMCA-2, PMCA-3 and PMCA-4.

71. The method of claim 69 wherein the antibody comprises an anti-PMCA-1 antibody, an anti-PMCA-2 antibody, an anti-PMCA-3 antibody, and an anti-PMCA-4 antibody.

72. The method of claim 69 wherein the antibody comprises a detectable label and the detecting includes detecting the detectable label.

73. The method of claim 69 wherein the antibody is a primary antibody and wherein the primary antibody is detected by a secondary antibody that binds to the primary antibody.

74. The method of claim 69 wherein the antibody is a polyclonal or a monoclonal antibody.

75. The method of claim 69 further comprises detecting cytocapsulars, cytocapsulasome vesicles, cytocapsular oncocells, cytocapsular tumorspheres, and cytocapsular tumorsphere network systems.
